(19) United States
(12) Patent Application Publication
(10) Pub. No.: US 2024/0092902 A1
(43) Pub. Date: Mar. 21, 2024

(54) METHODS FOR TREATING CANCER OR INFECTION USING A COMBINATION OF AN ANTI-PD-1 ANTIBODY, AN ANTI-LAG3 ANTIBODY, AND AN ANTI-TIGIT ANTIBODY

(71) Applicant: Merck Sharp & Dohme LLC, Rahway, NJ (US)

(72) Inventors: Mingmei Cai, Westwood, MA (US); Rene de Waal Malefyt, Sunnyvale, CA (US); Laurence Fayadat-Dilman, San Carlos, CA (US); Ying Li, Sunnyvale, CA (US); Ruban Mangadu, Livermore, CA (US); Uyen Phan, San Jose, CA (US)

(73) Assignee: Merck Sharp & Dohme LLC, Rahway, NJ (US)

(21) Appl. No.: 18/461,678

(22) Filed: Sep. 6, 2023

Related U.S. Application Data
(63) Continuation of application No. 16/966,100, filed on Jul. 30, 2020, now abandoned.
(60) Provisional application No. 62/625,025, filed on Feb. 1, 2018.

(51) Int. Cl. C07K 16/28; A61P 35/00

(52) U.S. Cl. CPC C07K 16/2803; A61P 35/00; C07K 16/2818; C07K 16/2896; A61K 2039/507

(57) ABSTRACT

Provided herein are methods of treating cancer or infection, which comprise administering to a human patient in need thereof: (a) an anti-human PD-1 monoclonal antibody or antigen binding fragment thereof; (b) an anti-human LAG3 monoclonal antibody or antigen binding fragment thereof; and (c) an anti-human TIGIT monoclonal antibody or antigen binding fragment thereof. Also provided are pharmaceutical compositions and kits containing such agents for the treatment of cancer or infection.

### CROSS REFERENCE TO RELATED APPLICATIONS

This application claims the benefit of priority to U.S. Provisional Application No. 62/625,025, filed Feb. 1, 2018, the disclosure of which is incorporated by reference in its entirety.

### I. FIELD

Provided herein are methods for treating cancer or an infectious disease using a combination of (a) an antibody that binds to a Programmed Death 1 protein (PD-1) or antigen binding fragment thereof, (b) an antibody that binds to a Lymphocyte-activation Gene 3 (LAG3) or antigen binding fragment thereof, and (c) an antibody that binds to a T cell immunoreceptor with Ig and ITIM domains (TIGIT) or antigen binding fragment thereof.

### II. BACKGROUND OF THE INVENTION

As with chronic viral infection, tumor antigen-specific CD4+ and CD8+ T cells display impaired effector function and an exhausted phenotype characterized by decreased production of pro-inflammatory cytokines and hyporesponsiveness to antigenic re-stimulation. This is mediated by cell extrinsic mechanisms, such as regulatory T cells (Treg), and cell intrinsic mechanisms, such as inhibitory molecules that are upregulated on exhausted, tumor-infiltrating lymphocytes. These inhibitory mechanisms represent a formidable barrier to effective antitumor immunity.

PD-1 is recognized as an important player in immune regulation and the maintenance of peripheral tolerance. Immune checkpoint therapies targeting PD-1 or its ligand (e.g., PD-L1) have resulted in groundbreaking improvements in clinical response in multiple human cancer types (Brahmer et al., N Engl J Med, 366: 2455-2465 (2012); Garon et al., N Engl J Med, 372:2018-2028 (2015); Hamid et al., N Engl J Med, 369:134-144 (2013); Robert et al., Lancet, 384:1109-1117 (2014); Robert et al., N Engl J Med, 372: 2521-2532 (2015); Robert et al., N Engl J Med, 372:320-330 (2015); Topalian et al., N Engl J Med, 366:2443-2454 (2012); Topalian et al., J Clin Oncol, 32:1020-1030 (2014); Wolchok et al., N Engl J Med, 369:122-133 (2013)). Immune therapies targeting the PD-1 axis include monoclonal antibodies directed to the PD-1 receptor (e.g., KEYTRUDA® (pembrolizumab), Merck and Co., Inc., Kenilworth, NJ; OPDIVO® (nivolumab), Bristol-Myers Squibb Company, Princeton, NJ) and those that bind to the PD-L1 ligand (e.g., TECENTRIQ® (atezolizumab), Genentech, San Francisco, CA).

LAG3 (CD223) is a cell surface molecule expressed on activated T cells, NK cells, B cells, and plasmacytoid dendritic cells and plays an important role in the function of these lymphocyte subsets (Huard et al., Immunogenetics, 39:213-217 (1994); Triebel et al., J Exp Med, 171:1393-1405 (1990); Kisielow et al., Eur J Immunol, 35:2081-2088 (2005); Workman et al., J Immunol, 182:1885-1891 (2009)). The interaction between LAG3 and its major ligand, Class II MHC, is thought to play a role in Treg suppressive function (Huang et al., Immunity 21 (4):503-13, (2004)). Recent preclinical studies have documented a role for LAG-3 in CD8+ T-cell exhaustion (Blackburn et al., Nat Immunol, 10:29-37 (2009)). Inhibition of LAG3 may lead to enhanced activation of antigen-specific T cells from which a therapeutic benefit may be gained.

TIGIT is an immunomodulatory receptor expressed primarily on activated T cells and NK cells (Yu et al., Nat Immunol, 10(1):48-57 (2009)). TIGIT forms part of a co-stimulatory network that consists of positive (such as CD226) and negative (such as TIGIT) immunomodulatory receptors on T cells, and ligands expressed on antigen-presenting cells (such as CD155 and CD112). Ligation of TIGIT by its ligands CD155 and CD112 expressed on tumor cells or tumor-associated macrophages may contribute to the suppression of T cell receptor signaling and T cell activation, which is essential for mounting effective anti-tumor immunity.

It has been proposed that the efficacy of anti-PD-1 or anti-PD-L1 antibodies might be enhanced if administered in combination with other approved or experimental cancer therapies, e.g., radiation, surgery, chemotherapeutic agents, targeted therapies, agents that inhibit other signaling pathways that are disregulated in tumors, and other immune enhancing agents. However, there are no clear guidelines as to which agent combined with the anti-PD-1 or anti-PD-L 1 antibodies may be effective or in which cancer types the combination may enhance the efficacy of treatment. Thus, there is an unmet need in the art for high efficacy therapeutic combinations that can generate a robust immune response to cancer.

### III. SUMMARY

The present disclosure provides methods of treating cancer (e.g., colorectal cancer) or an infectious disease (e.g., a viral infection) using a combination of an anti-PD-1 monoclonal antibody or antigen binding fragment thereof, an anti-LAG3 monoclonal antibody or antigen binding fragment thereof, and an anti-TIGIT monoclonal antibody or antigen binding fragment thereof.

The present disclosure also provides pharmaceutical compositions comprising an anti-PD-1 monoclonal antibody or antigen binding fragment thereof, an anti-LAG3 monoclonal antibody or antigen binding fragment thereof, and an anti-TIGIT monoclonal antibody or antigen binding fragment thereof.

The present disclosure further provides kits including an anti-PD-1 monoclonal antibody or antigen binding fragment thereof, an anti-LAG3 monoclonal antibody or antigen binding fragment thereof, and an anti-TIGIT monoclonal antibody or antigen binding fragment thereof.

Also provided herein are uses of a therapeutic combination for treating cancer (e.g., colorectal cancer), and the therapeutic combination includes an anti-PD-1 monoclonal antibody or antigen binding fragment thereof, an anti-LAG3 monoclonal antibody or antigen binding fragment thereof, and an anti-TIGIT monoclonal antibody or antigen binding fragment thereof.

In one aspect, provided herein is a method of treating cancer, comprising administering to a human patient in need thereof:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In some embodiments, the cancer is selected from the group consisting of osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer (e.g., non-small cell lung cancer), gastric cancer, colorectal cancer, cervical cancer, synovial sarcoma, head and neck cancer, squamous cell carcinoma, lymphoma (e.g., diffuse large B-cell lymphoma (DLBCL) or non-Hodgkin lymphoma (NHL)), multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, and carcinoid cancer.

In certain embodiments, the cancer is metastatic. In some embodiments, the cancer is relapsed. In other embodiments, the cancer is refractory. In yet other embodiments, the cancer is relapsed and refractory.

In one embodiment, the cancer is osteosarcoma. In another embodiment, the cancer is rhabdomyosarcoma. In yet another embodiment, the cancer is neuroblastoma. In still another embodiment, the cancer is kidney cancer. In one embodiment, the cancer is leukemia. In another embodiment, the cancer is renal transitional cell cancer. In yet another embodiment, the cancer is bladder cancer. In still another embodiment, the cancer is Wilm's cancer. In one embodiment, the cancer is ovarian cancer. In another embodiment, the cancer is pancreatic cancer. In yet another embodiment, the cancer is breast cancer. In still another embodiment, the cancer is prostate cancer. In one embodiment, the cancer is bone cancer. In another embodiment, the cancer is lung cancer. In yet another embodiment, the cancer is non-small cell lung cancer. In still another embodiment, the cancer is gastric cancer. In one embodiment, the cancer is colorectal cancer. In another embodiment, the cancer is cervical cancer. In yet another embodiment, the cancer is synovial sarcoma. In still another embodiment, the cancer is head and neck cancer. In one embodiment, the cancer is squamous cell carcinoma. In another embodiment, the cancer is lymphoma. In one embodiment, the cancer is DLBCL. In another embodiment, the cancer is NHL. In yet another embodiment, the cancer is multiple myeloma. In still another embodiment, the cancer is renal cell cancer. In one embodiment, the cancer is retinoblastoma. In another embodiment, the cancer is hepatoblastoma. In yet another embodiment, the cancer is hepatocellular carcinoma. In still another embodiment, the cancer is melanoma. In one embodiment, the cancer is rhabdoid tumor of the kidney. In another embodiment, the cancer is Ewing's sarcoma. In yet another embodiment, the cancer is chondrosarcoma. In still another embodiment, the cancer is brain cancer. In one embodiment, the cancer is glioblastoma. In another embodiment, the cancer is meningioma. In yet another embodiment, the cancer is pituitary adenoma. In still another embodiment, the cancer is vestibular schwannoma. In one embodiment, the cancer is primitive neuroectodermal tumor. In another embodiment, the cancer is medulloblastoma. In yet another embodiment, the cancer is astrocytoma. In still another embodiment, the cancer is anaplastic astrocytoma. In one embodiment, the cancer is oligodendroglioma. In another embodiment, the cancer is ependymoma. In yet another embodiment, the cancer is choroid plexus papilloma. In still another embodiment, the cancer is polycythemia vera. In one embodiment, the cancer is thrombocythemia. In another embodiment, the cancer is idiopathic myelfibrosis. In yet another embodiment, the cancer is soft tissue sarcoma. In still another embodiment, the cancer is thyroid cancer. In one embodiment, the cancer is endometrial cancer. In another embodiment, the cancer is carcinoid cancer. In yet another embodiment, the cancer is refractory head and neck cancer. In still another embodiment, the cancer is relapsed/refractory NHL (rrNHL). In yet still another embodiment, the cancer is PD-1 refractory rrNHL.

In another aspect, provided herein is a method of enhancing T cell activity, comprising contacting the T cells with:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In some embodiments, the enhancement of T cell activity occurs in vitro. In other embodiments, the enhancement of T cell activity occurs in vivo.

In certain embodiments, the enhancement of T cell activity is measured by increased cytokine production. In other embodiments, the enhancement of T cell activity is measured by increased cell proliferation.

Thus, in some embodiments, provided herein is a method of increasing cytokine production of T cells, comprising contacting the T cells with:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In some embodiments, the increased cytokine production of T cells occurs in vitro. In other embodiments, the increased cytokine production of T cells occurs in vivo.

In certain embodiments, the cytokine is selected from the group consisting of IL-1, IL-2, IL-6, IL-12, IL-17, IL-22, IL-23, GM-CSF, IFN-□, and TNF-□. In one embodiment, the cytokine is IL-1. In another embodiment, the cytokine is IL-2. In yet another embodiment, the cytokine is IL-6. In still another embodiment, the cytokine is IL-12. In one embodiment, the cytokine is IL-17. In another embodiment, the cytokine is IL-22. In yet another embodiment, the cytokine is IL-23. In still another embodiment, the cytokine is GM-CSF. In one embodiment, the cytokine is IFN-□. In another embodiment, the cytokine is TNF-□. In some embodiments, the cytokine is one, two, three, four, five, six, seven, eight, nine, or ten cytokines selected from the group consisting of IL-1, IL-2, IL-6, IL-12, IL-17, IL-22, IL-23, GM-CSF, IFN-□, and TNF-□.

In other embodiments, provided herein is a method of increasing proliferation of T cells, comprising contacting the T cells with:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In some embodiments, the increased proliferation of T cells occurs in vitro. In other embodiments, the increased proliferation of T cells occurs in vivo.

In another aspect, provided herein is a pharmaceutical composition comprising:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In certain embodiments, the pharmaceutical composition further comprises a pharmaceutically acceptable carrier.

In yet another aspect, provided herein is a kit comprising:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In certain embodiments, the kit further comprises instructions for administering to a human patient the anti-human PD-1 monoclonal antibody or antigen binding fragment thereof, the anti-human LAG3 monoclonal antibody or antigen binding fragment thereof, and the anti-human TIGIT monoclonal antibody or antigen binding fragment thereof.

In still another aspect, provided herein is use of a therapeutic combination for treating cancer in a human patient, wherein the therapeutic combination comprises:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In some embodiments, the cancer is selected from the group consisting of osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer (e.g., non-small cell lung cancer), gastric cancer, colorectal cancer, cervical cancer, synovial sarcoma, head and neck cancer, squamous cell carcinoma, lymphoma (e.g., DLBCL or NHL), multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, and carcinoid cancer.

In certain embodiments, the cancer is metastatic. In some embodiments, the cancer is relapsed. In other embodiments, the cancer is refractory. In yet other embodiments, the cancer is relapsed and refractory.

In one embodiment, the cancer is osteosarcoma. In another embodiment, the cancer is rhabdomyosarcoma. In yet another embodiment, the cancer is neuroblastoma. In still another embodiment, the cancer is kidney cancer. In one embodiment, the cancer is leukemia. In another embodiment, the cancer is renal transitional cell cancer. In yet another embodiment, the cancer is bladder cancer. In still another embodiment, the cancer is Wilm's cancer. In one embodiment, the cancer is ovarian cancer. In another embodiment, the cancer is pancreatic cancer. In yet another embodiment, the cancer is breast cancer. In still another embodiment, the cancer is prostate cancer. In one embodiment, the cancer is bone cancer. In another embodiment, the cancer is lung cancer. In yet another embodiment, the cancer is non-small cell lung cancer. In still another embodiment, the cancer is gastric cancer. In one embodiment, the cancer is colorectal cancer. In another embodiment, the cancer is cervical cancer. In yet another embodiment, the cancer is synovial sarcoma. In still another embodiment, the cancer is head and neck cancer. In one embodiment, the cancer is squamous cell carcinoma. In another embodiment, the cancer is lymphoma. In one embodiment, the cancer is DLBCL. In another embodiment, the cancer is NHL. In yet another embodiment, the cancer is multiple myeloma. In still another embodiment, the cancer is renal cell cancer. In one embodiment, the cancer is retinoblastoma. In another embodiment, the cancer is hepatoblastoma. In yet another embodiment, the cancer is hepatocellular carcinoma. In still another embodiment, the cancer is melanoma. In one embodiment, the cancer is rhabdoid tumor of the kidney. In another embodiment, the cancer is Ewing's sarcoma. In yet another embodiment, the cancer is chondrosarcoma. In still another embodiment, the cancer is brain cancer. In one embodiment, the cancer is glioblastoma. In another embodiment, the cancer is meningioma. In yet another embodiment, the cancer is pituitary adenoma. In still another embodiment, the cancer is vestibular schwannoma. In one embodiment, the cancer is primitive neuroectodermal tumor. In another embodiment, the cancer is medulloblastoma. In yet another embodiment, the cancer is astrocytoma. In still another embodiment, the cancer is anaplastic astrocytoma. In one embodiment, the cancer is oligodendroglioma. In another embodiment, the cancer is ependymoma. In yet another embodiment, the cancer is choroid plexus papilloma. In still another embodiment, the cancer is polycythemia vera. In one embodiment, the cancer is thrombocythemia. In another embodiment, the cancer is idiopathic myelfibrosis. In yet another embodiment, the cancer is soft tissue sarcoma. In still another embodiment, the cancer is thyroid cancer. In one embodiment, the cancer is endometrial cancer. In another embodiment, the cancer is carcinoid cancer. In yet another embodiment, the cancer is refractory head and neck cancer. In still another embodiment, the cancer is relapsed/refractory NHL (rrNHL). In yet still another embodiment, the cancer is PD-1 refractory rrNHL.

In certain embodiments of various methods, pharmaceutical compositions, kits, or uses provided herein, the anti-human PD-1 monoclonal antibody is a human antibody.

In some embodiments of various methods, pharmaceutical compositions, kits, or uses provided herein, the anti-human PD-1 monoclonal antibody is a humanized antibody.

In other embodiments of various methods, pharmaceutical compositions, kits, or uses provided herein, the anti-human LAG3 monoclonal antibody is a human antibody.

In yet other embodiments of various methods, pharmaceutical compositions, kits, or uses provided herein, the anti-human LAG3 monoclonal antibody is a humanized antibody.

In still other embodiments of various methods, pharmaceutical compositions, kits, or uses provided herein, the anti-human TIGIT monoclonal antibody is a human antibody.

In still other embodiments of various methods, pharmaceutical compositions, kits, or uses provided herein, the anti-human TIGIT monoclonal antibody is a humanized antibody.

In certain embodiments of various methods, pharmaceutical compositions, kits, or uses provided herein, the anti-human PD-1 monoclonal antibody or antigen binding fragment thereof comprises a light chain variable region (V_{L}) complementarity determining region 1 (CDR1), a V_{L} CDR2, and a V_{L} CDR3 comprising amino acid sequences as set forth in SEQ ID NOS:1, 2, and 3, respectively, and a heavy chain variable region (V_{H}) CDR1, a V_{H} CDR2, and a V_{H} CDR3 comprising amino acid sequences as set forth in SEQ ID NOS:6, 7, and 8, respectively.

In some embodiments of various methods, pharmaceutical compositions, kits, or uses provided herein, the anti-human PD-1 monoclonal antibody or antigen binding fragment thereof comprises a V_{L} region comprising an amino acid sequence as set forth in SEQ ID NO:4, and a V_{H} region comprising an amino acid sequence as set forth in SEQ ID NO:9.

In other embodiments of various methods, pharmaceutical compositions, kits, or uses provided herein, the anti-human PD-1 monoclonal antibody or antigen binding fragment thereof comprises a light chain comprising or consisting of an amino acid sequence as set forth in SEQ ID NO:5 and a heavy chain comprising or consisting of an amino acid sequence as set forth in SEQ ID NO:10.

In one embodiment of various methods, pharmaceutical compositions, kits, or uses provided herein, the anti-human PD-1 monoclonal antibody or antigen binding fragment thereof is pembrolizumab.

In another embodiment of various methods, pharmaceutical compositions, kits, or uses provided herein, the anti-human PD-1 monoclonal antibody or antigen binding fragment thereof is nivolumab.

In another embodiment of various methods, pharmaceutical compositions, kits, or uses provided herein, the anti-human PD-1 monoclonal antibody or antigen binding fragment thereof is cemiplimab.

In yet another embodiment of various methods, pharmaceutical compositions, kits, or uses provided herein, the anti-human PD-1 monoclonal antibody or antigen binding fragment thereof is pidilizumab (U.S. Pat. No. 7,332,582).

In one embodiment of various methods, pharmaceutical compositions, kits, or uses provided herein, the anti-human PD-1 monoclonal antibody or antigen binding fragment thereof is AMP-514 (MedImmune LLC, Gaithersburg, MD).

In another embodiment of various methods, pharmaceutical compositions, kits, or uses provided herein, the anti-human PD-1 monoclonal antibody or antigen binding fragment thereof is PDR001 (U.S. Pat. No. 9,683,048).

In yet another embodiment of various methods, pharmaceutical compositions, kits, or uses provided herein, the anti-human PD-1 monoclonal antibody or antigen binding fragment thereof is BGB-A317 (U.S. Pat. No. 8,735,553).

In still another embodiment of various methods, pharmaceutical compositions, kits, or uses provided herein, the anti-human PD-1 monoclonal antibody or antigen binding fragment thereof is MGA012 (MacroGenics, Rockville, MD).

In certain embodiments of various methods, pharmaceutical compositions, kits, or uses provided herein, the anti-human LAG3 monoclonal antibody or antigen binding fragment thereof comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively.

In some embodiments of various methods, pharmaceutical compositions, kits, or uses provided herein, the anti-human LAG3 monoclonal antibody or antigen binding fragment thereof comprises a V_{L} region comprising an amino acid sequence as set forth in SEQ ID NO:14, and a V_{H} region comprising an amino acid sequence as set forth in SEQ ID NO:19.

In other embodiments of various methods, pharmaceutical compositions, kits, or uses provided herein, the anti-human LAG3 monoclonal antibody or antigen binding fragment thereof comprises a light chain comprising or consisting of an amino acid sequence as set forth in SEQ ID NO:15 and a heavy chain comprising or consisting of an amino acid sequence as set forth in SEQ ID NO:20.

In one embodiment of various methods, pharmaceutical compositions, kits, or uses provided herein, the anti-human LAG3 monoclonal antibody or antigen binding fragment thereof is BMS-986016 (Bristol-Myers Squibb, New York, NY).

In another embodiment of various methods, pharmaceutical compositions, kits, or uses provided herein, the anti-human LAG3 monoclonal antibody or antigen binding fragment thereof is REGN3767 (Regeneron, Tarrytown, NY).

In yet another embodiment of various methods, pharmaceutical compositions, kits, or uses provided herein, the anti-human LAG3 monoclonal antibody or antigen binding fragment thereof is LAG525 (Novartis, Basel, Switzerland).

In still another embodiment of various methods, pharmaceutical compositions, kits, or uses provided herein, the anti-human LAG3 monoclonal antibody or antigen binding fragment thereof is GSK2813781 (GlaxoSmithKline, Brentford, UK).

In certain embodiments of various methods, pharmaceutical compositions, kits, or uses provided herein, the anti-human TIGIT monoclonal antibody or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In some embodiments of various methods, pharmaceutical compositions, kits, or uses provided herein, the anti-human TIGIT monoclonal antibody or antigen binding fragment thereof comprises a V_{L} region comprising an amino acid sequence as set forth in SEQ ID NO:24, and a V_{H} region comprising an amino acid sequence as set forth in SEQ ID NO:29.

In other embodiments of various methods, pharmaceutical compositions, kits, or uses provided herein, the anti-human TIGIT monoclonal antibody or antigen binding fragment thereof comprises a light chain comprising or consisting of an amino acid sequence as set forth in SEQ ID NO:25 and a heavy chain comprising or consisting of an amino acid sequence as set forth in SEQ ID NO:30.

In one embodiment of various methods, pharmaceutical compositions, kits, or uses provided herein, the anti-human TIGIT monoclonal antibody or antigen binding fragment thereof is BMS-986207 (Bristol-Myers Squibb, New York, NY).

In another embodiment of various methods, pharmaceutical compositions, kits, or uses provided herein, the anti-human TIGIT monoclonal antibody or antigen binding fragment thereof is OMP-313M32 (OncoMed Pharmaceuticals, Redwood city, CA).

In yet another embodiment of various methods, pharmaceutical compositions, kits, or uses provided herein, the anti-human TIGIT monoclonal antibody or antigen binding fragment thereof is MTIG7192A (also known as RG6058, U.S. Publ. No. 2017/0088613).

In still another embodiment of various methods, pharmaceutical compositions, kits, or uses provided herein, the anti-human TIGIT monoclonal antibody or antigen binding fragment thereof is PTZ-201 (Potenza Therapeutics, Cambridge, MA; also known as ASP8374, Astellas Pharma, Tokyo, Japan).

In one specific embodiment of various methods, pharmaceutical compositions, kits, or uses provided herein, the anti-human PD-1 monoclonal antibody or antigen binding fragment thereof comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; the anti-human LAG3 monoclonal antibody or antigen binding fragment thereof comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and the anti-human TIGIT monoclonal antibody or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In another specific embodiment of various methods, pharmaceutical compositions, kits, or uses provided herein, the anti-human PD-1 monoclonal antibody or antigen binding fragment thereof comprises a V_{L} region comprising an amino acid sequence as set forth in SEQ ID NO:4, and a V_{H} region comprising an amino acid sequence as set forth in SEQ ID NO:9; the anti-human LAG3 monoclonal antibody or antigen binding fragment thereof comprises a V_{L} region comprising an amino acid sequence as set forth in SEQ ID NO:14, and a V_{H} region comprising an amino acid sequence as set forth in SEQ ID NO:19; and the anti-human TIGIT monoclonal antibody or antigen binding fragment thereof comprises a V_{L} region comprising an amino acid sequence as set forth in SEQ ID NO:24, and a V_{H} region comprising an amino acid sequence as set forth in SEQ ID NO:29.

In yet another specific embodiment of various methods, pharmaceutical compositions, kits, or uses provided herein, the anti-human PD-1 monoclonal antibody or antigen binding fragment thereof comprises a light chain comprising or consisting of an amino acid sequence as set forth in SEQ ID NO:5 and a heavy chain comprising or consisting of an amino acid sequence as set forth in SEQ ID NO:10; the anti-human LAG3 monoclonal antibody or antigen binding fragment thereof comprises a light chain comprising or consisting of an amino acid sequence as set forth in SEQ ID NO:15 and a heavy chain comprising or consisting of an amino acid sequence as set forth in SEQ ID NO:20; and the anti-human TIGIT monoclonal antibody or antigen binding fragment thereof comprises a light chain comprising or consisting of an amino acid sequence as set forth in SEQ ID NO:25 and a heavy chain comprising or consisting of an amino acid sequence as set forth in SEQ ID NO:30.

In one specific embodiment of various methods, pharmaceutical compositions, kits, or uses provided herein, the anti-human PD-1 monoclonal antibody or antigen binding fragment thereof comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; the anti-human LAG3 monoclonal antibody or antigen binding fragment thereof comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and the anti-human TIGIT monoclonal antibody or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In another specific embodiment of various methods, pharmaceutical compositions, kits, or uses provided herein, the anti-human PD-1 monoclonal antibody or antigen binding fragment thereof comprises a V_{L} region comprising an amino acid sequence as set forth in SEQ ID NO:34, and a V_{H} region comprising an amino acid sequence as set forth in SEQ ID NO:39; the anti-human LAG3 monoclonal antibody or antigen binding fragment thereof comprises a V_{L} region comprising an amino acid sequence as set forth in SEQ ID NO:14, and a V_{H} region comprising an amino acid sequence as set forth in SEQ ID NO:19; and the anti-human TIGIT monoclonal antibody or antigen binding fragment thereof comprises a V_{L} region comprising an amino acid sequence as set forth in SEQ ID NO:24, and a V_{H} region comprising an amino acid sequence as set forth in SEQ ID NO:29.

In yet another specific embodiment of various methods, pharmaceutical compositions, kits, or uses provided herein, the anti-human PD-1 monoclonal antibody or antigen binding fragment thereof comprises a light chain comprising or consisting of an amino acid sequence as set forth in SEQ ID NO:35 and a heavy chain comprising or consisting of an amino acid sequence as set forth in SEQ ID NO:40; the anti-human LAG3 monoclonal antibody or antigen binding fragment thereof comprises a light chain comprising or consisting of an amino acid sequence as set forth in SEQ ID NO:15 and a heavy chain comprising or consisting of an amino acid sequence as set forth in SEQ ID NO:20; and the anti-human TIGIT monoclonal antibody or antigen binding fragment thereof comprises a light chain comprising or consisting of an amino acid sequence as set forth in SEQ ID NO:25 and a heavy chain comprising or consisting of an amino acid sequence as set forth in SEQ ID NO:30.

In one specific embodiment of various methods, pharmaceutical compositions, kits, or uses provided herein, the anti-human PD-1 monoclonal antibody or antigen binding fragment thereof is cemiplimab; the anti-human LAG3 monoclonal antibody or antigen binding fragment thereof comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and the anti-human TIGIT monoclonal antibody or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment of various methods, pharmaceutical compositions, kits, or uses provided herein, the anti-human PD-1 monoclonal antibody or antigen binding fragment thereof is pidilizumab; the anti-human LAG3 monoclonal antibody or antigen binding fragment thereof comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and the anti-human TIGIT monoclonal antibody or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment of various methods, pharmaceutical compositions, kits, or uses provided herein, the anti-human PD-1 monoclonal antibody or antigen binding fragment thereof is AMP-514; the anti-human LAG3 monoclonal antibody or antigen binding fragment thereof comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and the anti-human TIGIT monoclonal antibody or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment of various methods, pharmaceutical compositions, kits, or uses provided herein, the anti-human PD-1 monoclonal antibody or antigen binding fragment thereof is PDR001; the anti-human LAG3 monoclonal antibody or antigen binding fragment thereof comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and the anti-human TIGIT monoclonal antibody or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment of various methods, pharmaceutical compositions, kits, or uses provided herein, the anti-human PD-1 monoclonal antibody or antigen binding fragment thereof is BGB-A317; the anti-human LAG3 monoclonal antibody or antigen binding fragment thereof comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and the anti-human TIGIT monoclonal antibody or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment of various methods, pharmaceutical compositions, kits, or uses provided herein, the anti-human PD-1 monoclonal antibody or antigen binding fragment thereof is MGA012; the anti-human LAG3 monoclonal antibody or antigen binding fragment thereof comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and the anti-human TIGIT monoclonal antibody or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment of various methods, pharmaceutical compositions, kits, or uses provided herein, the anti-human PD-1 monoclonal antibody or antigen binding fragment thereof comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; the anti-human LAG3 monoclonal antibody or antigen binding fragment thereof is BMS-986016; and the anti-human TIGIT monoclonal antibody or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment of various methods, pharmaceutical compositions, kits, or uses provided herein, the anti-human PD-1 monoclonal antibody or antigen binding fragment thereof comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; the anti-human LAG3 monoclonal antibody or antigen binding fragment thereof is REGN3767; and the anti-human TIGIT monoclonal antibody or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment of various methods, pharmaceutical compositions, kits, or uses provided herein, the anti-human PD-1 monoclonal antibody or antigen binding fragment thereof comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; the anti-human LAG3 monoclonal antibody or antigen binding fragment thereof is LAG525; and the anti-human TIGIT monoclonal antibody or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment of various methods, pharmaceutical compositions, kits, or uses provided herein, the anti-human PD-1 monoclonal antibody or antigen binding fragment thereof comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; the anti-human LAG3 monoclonal antibody or antigen binding fragment thereof is GSK2813781; and the anti-human TIGIT monoclonal antibody or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment of various methods, pharmaceutical compositions, kits, or uses provided herein, the anti-human PD-1 monoclonal antibody or antigen binding fragment thereof comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; the anti-human LAG3 monoclonal antibody or antigen binding fragment thereof is BMS-986016; and the anti-human TIGIT monoclonal antibody or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment of various methods, pharmaceutical compositions, kits, or uses provided herein, the anti-human PD-1 monoclonal antibody or antigen binding fragment thereof comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; the anti-human LAG3 monoclonal antibody or antigen binding fragment thereof is REGN3767; and the anti-human TIGIT monoclonal antibody or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment of various methods, pharmaceutical compositions, kits, or uses provided herein, the anti-human PD-1 monoclonal antibody or antigen binding fragment thereof comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; the anti-human LAG3 monoclonal antibody or antigen binding fragment thereof is LAG525; and the anti-human TIGIT monoclonal antibody or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment of various methods, pharmaceutical compositions, kits, or uses provided herein, the anti-human PD-1 monoclonal antibody or antigen binding fragment thereof comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; the anti-human LAG3 monoclonal antibody or antigen binding fragment thereof is GSK2813781; and the anti-human TIGIT monoclonal antibody or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment of various methods, pharmaceutical compositions, kits, or uses provided herein, the anti-human PD-1 monoclonal antibody or antigen binding fragment thereof comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; the anti-human LAG3 monoclonal antibody or antigen binding fragment thereof comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and the anti-human TIGIT monoclonal antibody or antigen binding fragment thereof is BMS-986207.

In one specific embodiment of various methods, pharmaceutical compositions, kits, or uses provided herein, the anti-human PD-1 monoclonal antibody or antigen binding fragment thereof comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; the anti-human LAG3 monoclonal antibody or antigen binding fragment thereof comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and the anti-human TIGIT monoclonal antibody or antigen binding fragment thereof is OMP-313M32.

In one specific embodiment of various methods, pharmaceutical compositions, kits, or uses provided herein, the anti-human PD-1 monoclonal antibody or antigen binding fragment thereof comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; the anti-human LAG3 monoclonal antibody or antigen binding fragment thereof comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and the anti-human TIGIT monoclonal antibody or antigen binding fragment thereof is MTIG7192A (RG6058).

In one specific embodiment of various methods, pharmaceutical compositions, kits, or uses provided herein, the anti-human PD-1 monoclonal antibody or antigen binding fragment thereof comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; the anti-human LAG3 monoclonal antibody or antigen binding fragment thereof comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; the anti-human LAG3 monoclonal antibody or antigen binding fragment thereof comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and the anti-human TIGIT monoclonal antibody or antigen binding fragment thereof is BMS-986207.

In one specific embodiment of various methods, pharmaceutical compositions, kits, or uses provided herein, the anti-human PD-1 monoclonal antibody or antigen binding fragment thereof comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; the anti-human LAG3 monoclonal antibody or antigen binding fragment thereof comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and the anti-human TIGIT monoclonal antibody or antigen binding fragment thereof is OMP-313M32.

In one specific embodiment of various methods, pharmaceutical compositions, kits, or uses provided herein, the anti-human PD-1 monoclonal antibody or antigen binding fragment thereof comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; the anti-human LAG3 monoclonal antibody or antigen binding fragment thereof comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and the anti-human TIGIT monoclonal antibody or antigen binding fragment thereof is MTIG7192A (RG6058).

In one specific embodiment of various methods, pharmaceutical compositions, kits, or uses provided herein, the anti-human PD-1 monoclonal antibody or antigen binding fragment thereof comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; the anti-human LAG3 monoclonal antibody or antigen binding fragment thereof comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is pembrolizumab, the anti-human LAG3 monoclonal antibody or antigen binding fragment thereof is BMS-986016, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is pembrolizumab, the anti-human LAG3 monoclonal antibody or antigen binding fragment thereof is BMS-986016, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is pembrolizumab, the anti-human LAG3 monoclonal antibody or antigen binding fragment thereof is BMS-986016, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is pembrolizumab, the anti-human LAG3 monoclonal antibody or antigen binding fragment thereof is BMS-986016, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is pembrolizumab, the anti-human LAG3 monoclonal antibody or antigen binding fragment thereof is REGN3767, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is pembrolizumab, the anti-human LAG3 monoclonal antibody or antigen binding fragment thereof is REGN3767, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is pembrolizumab, the anti-human LAG3 monoclonal antibody or antigen binding fragment thereof is REGN3767, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is pembrolizumab, the anti-human LAG3 monoclonal antibody or antigen binding fragment thereof is REGN3767, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is pembrolizumab, the anti-human LAG3 monoclonal antibody or antigen binding fragment thereof is LAG525, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is pembrolizumab, the anti-human LAG3 monoclonal antibody or antigen binding fragment thereof is LAG525, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is pembrolizumab, the anti-human LAG3 monoclonal antibody or antigen binding fragment thereof is LAG525, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is pembrolizumab, the anti-human LAG3 monoclonal antibody or antigen binding fragment thereof is LAG525, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is pembrolizumab, the anti-human LAG3 monoclonal antibody or antigen binding fragment thereof is GSK2813781, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is pembrolizumab, the anti-human LAG3 monoclonal antibody or antigen binding fragment thereof is GSK2813781, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is pembrolizumab, the anti-human LAG3 monoclonal antibody or antigen binding fragment thereof is GSK2813781, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is pembrolizumab, the anti-human LAG3 monoclonal antibody or antigen binding fragment thereof is GSK2813781, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is nivolumab, the anti-human LAG3 monoclonal antibody or antigen binding fragment thereof is BMS-986016, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is nivolumab, the anti-human LAG3 monoclonal antibody or antigen binding fragment thereof is BMS-986016, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is nivolumab, the anti-human LAG3 monoclonal antibody or antigen binding fragment thereof is BMS-986016, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is nivolumab, the anti-human LAG3 monoclonal antibody or antigen binding fragment thereof is BMS-986016, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is nivolumab, the anti-human LAG3 monoclonal antibody or antigen binding fragment thereof is REGN3767, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is nivolumab, the anti-human LAG3 monoclonal antibody or antigen binding fragment thereof is REGN3767, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is nivolumab, the anti-human LAG3 monoclonal antibody or antigen binding fragment thereof is REGN3767, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is nivolumab, the anti-human LAG3 monoclonal antibody or antigen binding fragment thereof is REGN3767, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is nivolumab, the anti-human LAG3 monoclonal antibody or antigen binding fragment thereof is LAG525, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is nivolumab, the anti-human LAG3 monoclonal antibody or antigen binding fragment thereof is LAG525, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is nivolumab, the anti-human LAG3 monoclonal antibody or antigen binding fragment thereof is LAG525, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is nivolumab, the anti-human LAG3 monoclonal antibody or antigen binding fragment thereof is LAG525, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is nivolumab, the anti-human LAG3 monoclonal antibody or antigen binding fragment thereof is GSK2813781, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is nivolumab, the anti-human LAG3 monoclonal antibody or antigen binding fragment thereof is GSK2813781, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is nivolumab, the anti-human LAG3 monoclonal antibody or antigen binding fragment thereof is GSK2813781, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is nivolumab, the anti-human LAG3 monoclonal antibody or antigen binding fragment thereof is GSK2813781, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is cemiplimab, the anti-human LAG3 monoclonal antibody or antigen binding fragment thereof is BMS-986016, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is cemiplimab, the anti-human LAG3 monoclonal antibody or antigen binding fragment thereof is BMS-986016, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is cemiplimab, the anti-human LAG3 monoclonal antibody or antigen binding fragment thereof is BMS-986016, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is cemiplimab, the anti-human LAG3 monoclonal antibody or antigen binding fragment thereof is BMS-986016, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is cemiplimab, the anti-human LAG3 monoclonal antibody or antigen binding fragment thereof is REGN3767, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is cemiplimab, the anti-human LAG3 monoclonal antibody or antigen binding fragment thereof is REGN3767, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is cemiplimab, the anti-human LAG3 monoclonal antibody or antigen binding fragment thereof is REGN3767, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is cemiplimab, the anti-human LAG3 monoclonal antibody or antigen binding fragment thereof is REGN3767, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is cemiplimab, the anti-human LAG3 monoclonal antibody or antigen binding fragment thereof is LAG525, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is cemiplimab, the anti-human LAG3 monoclonal antibody or antigen binding fragment thereof is LAG525, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is cemiplimab, the anti-human LAG3 monoclonal antibody or antigen binding fragment thereof is LAG525, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is cemiplimab, the anti-human LAG3 monoclonal antibody or antigen binding fragment thereof is LAG525, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is cemiplimab, the anti-human LAG3 monoclonal antibody or antigen binding fragment thereof is GSK2813781, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is cemiplimab, the anti-human LAG3 monoclonal antibody or antigen binding fragment thereof is GSK2813781, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is cemiplimab, the anti-human LAG3 monoclonal antibody or antigen binding fragment thereof is GSK2813781, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is cemiplimab, the anti-human LAG3 monoclonal antibody or antigen binding fragment thereof is GSK2813781, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is pidilizumab, the anti-human LAG3 monoclonal antibody or antigen binding fragment thereof is BMS-986016, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is pidilizumab, the anti-human LAG3 monoclonal antibody or antigen binding fragment thereof is BMS-986016, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is pidilizumab, the anti-human LAG3 monoclonal antibody or antigen binding fragment thereof is BMS-986016, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is pidilizumab, the anti-human LAG3 monoclonal antibody or antigen binding fragment thereof is BMS-986016, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is pidilizumab, the anti-human LAG3 monoclonal antibody or antigen binding fragment thereof is REGN3767, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is pidilizumab, the anti-human LAG3 monoclonal antibody or antigen binding fragment thereof is REGN3767, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is pidilizumab, the anti-human LAG3 monoclonal antibody or antigen binding fragment thereof is REGN3767, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is pidilizumab, the anti-human LAG3 monoclonal antibody or antigen binding fragment thereof is REGN3767, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is pidilizumab, the anti-human LAG3 monoclonal antibody or antigen binding fragment thereof is LAG525, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is pidilizumab, the anti-human LAG3 monoclonal antibody or antigen binding fragment thereof is LAG525, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is pidilizumab, the anti-human LAG3 monoclonal antibody or antigen binding fragment thereof is LAG525, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is pidilizumab, the anti-human LAG3 monoclonal antibody or antigen binding fragment thereof is LAG525, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is pidilizumab, the anti-human LAG3 monoclonal antibody or antigen binding fragment thereof is GSK2813781, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is pidilizumab, the anti-human LAG3 monoclonal antibody or antigen binding fragment thereof is GSK2813781, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is pidilizumab, the anti-human LAG3 monoclonal antibody or antigen binding fragment thereof is GSK2813781, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is pidilizumab, the anti-human LAG3 monoclonal antibody or antigen binding fragment thereof is GSK2813781, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is AMP-514, the anti-human LAG3 monoclonal antibody or antigen binding fragment thereof is BMS-986016, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is AMP-514, the anti-human LAG3 monoclonal antibody or antigen binding fragment thereof is BMS-986016, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is AMP-514, the anti-human LAG3 monoclonal antibody or antigen binding fragment thereof is BMS-986016, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is AMP-514, the anti-human LAG3 monoclonal antibody or antigen binding fragment thereof is BMS-986016, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is AMP-514, the anti-human LAG3 monoclonal antibody or antigen binding fragment thereof is REGN3767, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is AMP-514, the anti-human LAG3 monoclonal antibody or antigen binding fragment thereof is REGN3767, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is AMP-514, the anti-human LAG3 monoclonal antibody or antigen binding fragment thereof is REGN3767, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is AMP-514, the anti-human LAG3 monoclonal antibody or antigen binding fragment thereof is REGN3767, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is AMP-514, the anti-human LAG3 monoclonal antibody or antigen binding fragment thereof is LAG525, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is AMP-514, the anti-human LAG3 monoclonal antibody or antigen binding fragment thereof is LAG525, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is AMP-514, the anti-human LAG3 monoclonal antibody or antigen binding fragment thereof is LAG525, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is AMP-514, the anti-human LAG3 monoclonal antibody or antigen binding fragment thereof is LAG525, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is AMP-514, the anti-human LAG3 monoclonal antibody or antigen binding fragment thereof is GSK2813781, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is AMP-514, the anti-human LAG3 monoclonal antibody or antigen binding fragment thereof is GSK2813781, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is AMP-514, the anti-human LAG3 monoclonal antibody or antigen binding fragment thereof is GSK2813781, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is AMP-514, the anti-human LAG3 monoclonal antibody or antigen binding fragment thereof is GSK2813781, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is PDR001, the anti-human LAG3 monoclonal antibody or antigen binding fragment thereof is BMS-986016, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is PDR001, the anti-human LAG3 monoclonal antibody or antigen binding fragment thereof is BMS-986016, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is PDR001, the anti-human LAG3 monoclonal antibody or antigen binding fragment thereof is BMS-986016, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is PDR001, the anti-human LAG3 monoclonal antibody or antigen binding fragment thereof is BMS-986016, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is PDR001, the anti-human LAG3 monoclonal antibody or antigen binding fragment thereof is REGN3767, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is PDR001, the anti-human LAG3 monoclonal antibody or antigen binding fragment thereof is REGN3767, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is PDR001, the anti-human LAG3 monoclonal antibody or antigen binding fragment thereof is REGN3767, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is PDR001, the anti-human LAG3 monoclonal antibody or antigen binding fragment thereof is REGN3767, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is PDR001, the anti-human LAG3 monoclonal antibody or antigen binding fragment thereof is LAG525, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is PDR001, the anti-human LAG3 monoclonal antibody or antigen binding fragment thereof is LAG525, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is PDR001, the anti-human LAG3 monoclonal antibody or antigen binding fragment thereof is LAG525, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is PDR001, the anti-human LAG3 monoclonal antibody or antigen binding fragment thereof is LAG525, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is PDR001, the anti-human LAG3 monoclonal antibody or antigen binding fragment thereof is GSK2813781, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is PDR001, the anti-human LAG3 monoclonal antibody or antigen binding fragment thereof is GSK2813781, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is PDR001, the anti-human LAG3 monoclonal antibody or antigen binding fragment thereof is GSK2813781, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is PDR001, the anti-human LAG3 monoclonal antibody or antigen binding fragment thereof is GSK2813781, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is BGB-A317, the anti-human LAG3 monoclonal antibody or antigen binding fragment thereof is BMS-986016, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is BGB-A317, the anti-human LAG3 monoclonal antibody or antigen binding fragment thereof is BMS-986016, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is BGB-A317, the anti-human LAG3 monoclonal antibody or antigen binding fragment thereof is BMS-986016, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is BGB-A317, the anti-human LAG3 monoclonal antibody or antigen binding fragment thereof is BMS-986016, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is BGB-A317, the anti-human LAG3 monoclonal antibody or antigen binding fragment thereof is REGN3767, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is BGB-A317, the anti-human LAG3 monoclonal antibody or antigen binding fragment thereof is REGN3767, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is BGB-A317, the anti-human LAG3 monoclonal antibody or antigen binding fragment thereof is REGN3767, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is BGB-A317, the anti-human LAG3 monoclonal antibody or antigen binding fragment thereof is REGN3767, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is BGB-A317, the anti-human LAG3 monoclonal antibody or antigen binding fragment thereof is LAG525, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is BGB-A317, the anti-human LAG3 monoclonal antibody or antigen binding fragment thereof is LAG525, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is BGB-A317, the anti-human LAG3 monoclonal antibody or antigen binding fragment thereof is LAG525, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is BGB-A317, the anti-human LAG3 monoclonal antibody or antigen binding fragment thereof is LAG525, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is BGB-A317, the anti-human LAG3 monoclonal antibody or antigen binding fragment thereof is GSK2813781, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is BGB-A317, the anti-human LAG3 monoclonal antibody or antigen binding fragment thereof is GSK2813781, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is BGB-A317, the anti-human LAG3 monoclonal antibody or antigen binding fragment thereof is GSK2813781, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is BGB-A317, the anti-human LAG3 monoclonal antibody or antigen binding fragment thereof is GSK2813781, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is MGA012, the anti-human LAG3 monoclonal antibody or antigen binding fragment thereof is BMS-986016, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is MGA012, the anti-human LAG3 monoclonal antibody or antigen binding fragment thereof is BMS-986016, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is MGA012, the anti-human LAG3 monoclonal antibody or antigen binding fragment thereof is BMS-986016, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is MGA012, the anti-human LAG3 monoclonal antibody or antigen binding fragment thereof is BMS-986016, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is MGA012, the anti-human LAG3 monoclonal antibody or antigen binding fragment thereof is REGN3767, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is MGA012, the anti-human LAG3 monoclonal antibody or antigen binding fragment thereof is REGN3767, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is MGA012, the anti-human LAG3 monoclonal antibody or antigen binding fragment thereof is REGN3767, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is MGA012, the anti-human LAG3 monoclonal antibody or antigen binding fragment thereof is REGN3767, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is MGA012, the anti-human LAG3 monoclonal antibody or antigen binding fragment thereof is LAG525, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is MGA012, the anti-human LAG3 monoclonal antibody or antigen binding fragment thereof is LAG525, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is MGA012, the anti-human LAG3 monoclonal antibody or antigen binding fragment thereof is LAG525, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is MGA012, the anti-human LAG3 monoclonal antibody or antigen binding fragment thereof is LAG525, and the anti-human TIGIT monoclonal antibody or antigen binding fragment thereof is PTZ-201 (ASP8374).

In one specific embodiment of various methods, pharmaceutical compositions, kits, or uses provided herein, the anti-human PD-1 monoclonal antibody or antigen binding fragment thereof is MGA012, the anti-human LAG3 monoclonal antibody or antigen binding fragment thereof is GSK2813781, and the anti-human TIGIT monoclonal antibody or antigen binding fragment thereof is BMS-986207.

In another specific embodiment of various methods, pharmaceutical compositions, kits, or uses provided herein, the anti-human PD-1 monoclonal antibody or antigen binding fragment thereof is MGA012, the anti-human LAG3 monoclonal antibody or antigen binding fragment thereof is GSK2813781, and the anti-human TIGIT monoclonal antibody or antigen binding fragment thereof is OMP-313M32.

In yet another specific embodiment of various methods, pharmaceutical compositions, kits, or uses provided herein, the anti-human PD-1 monoclonal antibody or antigen binding fragment thereof is MGA012, the anti-human LAG3 monoclonal antibody or antigen binding fragment thereof is GSK2813781, and the anti-human TIGIT monoclonal antibody or antigen binding fragment thereof is MTIG7192A (RG6058).

In still another embodiment of various methods, pharmaceutical compositions, kits, or uses provided herein, the anti-human PD-1 monoclonal antibody or antigen binding fragment thereof is MGA012, the anti-human LAG3 monoclonal antibody or antigen binding fragment thereof is GSK2813781, and the anti-human TIGIT monoclonal antibody or antigen binding fragment thereof is PTZ-201 (ASP8374).

In some embodiments, the human patient is administered:
(a) 200 mg, 240 mg, or 2 mg/kg pembrolizumab;
(b) an anti-human LAG3 antibody or antigen binding fragment thereof; and
(c) an anti-human TIGIT antibody or antigen binding fragment thereof;
wherein pembrolizumab is administered once every three weeks.

In certain embodiments, the human patient is administered:
(a) 200 mg pembrolizumab;
(b) an anti-human LAG3 antibody or antigen binding fragment thereof; and
(c) an anti-human TIGIT antibody or antigen binding fragment thereof;
wherein pembrolizumab is administered once every three weeks.

In certain embodiments, the human patient is administered:
(a) 240 mg pembrolizumab;
(b) an anti-human LAG3 antibody or antigen binding fragment thereof; and
(c) an anti-human TIGIT antibody or antigen binding fragment thereof;
wherein pembrolizumab is administered once every three weeks.

In other embodiments, the human patient is administered:
(a) 2 mg/kg pembrolizumab;
(b) an anti-human LAG3 antibody or antigen binding fragment thereof; and
(c) an anti-human TIGIT antibody or antigen binding fragment thereof;
wherein pembrolizumab is administered once every three weeks.

In certain embodiments, the human patient is administered:
(a) 400 mg pembrolizumab;
(b) an anti-human LAG3 antibody or antigen binding fragment thereof; and
(c) an anti-human TIGIT antibody or antigen binding fragment thereof;
wherein pembrolizumab is administered once every six weeks.

In some embodiments, the human patient is administered:
(a) 240 mg or 3 mg/kg nivolumab;
(b) an anti-human LAG3 antibody or antigen binding fragment thereof; and
(c) an anti-human TIGIT antibody or antigen binding fragment thereof;
wherein nivolumab is administered once every two weeks.

In some embodiments, the human patient is administered:
(a) 240 mg nivolumab;
(b) an anti-human LAG3 antibody or antigen binding fragment thereof; and
(c) an anti-human TIGIT antibody or antigen binding fragment thereof;
wherein nivolumab is administered once every two weeks.

In some embodiments, the human patient is administered:
(a) 3 mg/kg nivolumab;
(b) an anti-human LAG3 antibody or antigen binding fragment thereof; and
(c) an anti-human TIGIT antibody or antigen binding fragment thereof;
wherein nivolumab is administered once every two weeks.

In some embodiments, the human patient is administered:
(a) 350 mg cemiplimab;
(b) an anti-human LAG3 antibody or antigen binding fragment thereof; and
(c) an anti-human TIGIT antibody or antigen binding fragment thereof;
wherein nivolumab is administered once every three weeks.

### IV. BRIEF DESCRIPTION OF THE DRAWINGS

FIGS. 1A-1F show tumor growth curves for individual animals and number of complete regressions (CRs) in syngeneic mouse colon carcinoma CT-26 model study. Group 1 animals were treated with isotype control monoclonal antibodies (A), Group 2 animals were treated with an anti-mouse PD-1 monoclonal antibody (B), Group 3 animals were treated with an anti-mouse PD-1 monoclonal antibody and an anti-mouse TIGIT monoclonal antibody (C), Group 4 animals were treated with an anti-mouse PD-1 monoclonal antibody and an anti-mouse LAG3 monoclonal antibody (D), Group 5 animals were treated with an anti-mouse TIGIT monoclonal antibody and an anti-mouse LAG3 monoclonal antibody (E), and Group 6 animals were treated with an anti-mouse PD-1 monoclonal antibody, an anti-mouse TIGIT monoclonal antibody, and an anti-mouse LAG3 monoclonal antibody (F). Dashed line indicates Day 0 mean tumor volume of 166 mm³.

FIGS. 2A-2H show tumor growth curves for individual animals and number of complete regressions (CRs) in syngeneic mouse bladder carcinoma MBT-2 model study. Group 1 animals were treated with isotype control monoclonal antibodies (A), Group 2 animals were treated with an anti-mouse PD-1 monoclonal antibody (B), Group 3 animals were treated with an anti-mouse TIGIT monoclonal antibody (C), Group 4 animals were treated with an anti-mouse LAG3 monoclonal antibody (D), Group 5 animals were treated with an anti-mouse PD-1 monoclonal antibody and an anti-mouse TIGIT monoclonal antibody (E), Group 6 animals were treated with an anti-mouse PD-1 monoclonal antibody and an anti-mouse LAG3 monoclonal antibody (F), Group 7 animals were treated with an anti-mouse TIGIT monoclonal antibody and an anti-mouse LAG3 monoclonal antibody (G), and Group 8 animals were treated with an anti-mouse PD-1 monoclonal antibody, an anti-mouse TIGIT monoclonal antibody, and an anti-mouse LAG3 monoclonal antibody (H). Dashed line indicates Day 0 mean tumor volume of 196 mm³.

FIGS. 3A-3H show tumor growth curves for individual animals and number of complete regressions (CRs) in syngeneic mouse renal carcinoma RENCA model study. Group 1 animals were treated with isotype control monoclonal antibodies (A), Group 2 animals were treated with an anti-mouse PD-1 monoclonal antibody (B), Group 3 animals were treated with an anti-mouse TIGIT monoclonal antibody (C), Group 4 animals were treated with an anti-mouse LAG3 monoclonal antibody (D), Group 5 animals were treated with an anti-mouse PD-1 monoclonal antibody and an anti-mouse TIGIT monoclonal antibody (E), Group 6 animals were treated with an anti-mouse PD-1 monoclonal antibody and an anti-mouse LAG3 monoclonal antibody (F), Group 7 animals were treated with an anti-mouse TIGIT monoclonal antibody and an anti-mouse LAG3 monoclonal antibody (G), and Group 8 animals were treated with an anti-mouse PD-1 monoclonal antibody, an anti-mouse TIGIT monoclonal antibody, and an anti-mouse LAG3 monoclonal antibody (H). Dashed line indicates Day 0 mean tumor volume of 205 mm³.

FIGS. 4A and 4B demonstrate that treatment with a triple combination of an anti-human PD-1 antibody, an anti-human LAG3 antibody, and an anti-human TIGIT antibody increased IFN-γ production in human CD4+ T cell clone 4-49, compared to treatment with individual antibody alone or combinations of any two antibodies. (A) depicts the antibody concentration-dependent curves of IFN-γ production from an exemplary experiment. (B) summarizes the IFN-γ levels at 67 nM antibody concentration in four independent experiments.

FIG. 5 demonstrate that treatment with a triple combination of an anti-human PD-1 antibody, an anti-human LAG3 antibody, and an anti-human TIGIT antibody increased cell proliferation of human CD4+ T cell clone 4-49, compared to treatment with individual antibody alone or combinations of any two antibodies.

### V. DETAILED DESCRIPTION OF THE INVENTION

1. Abbreviations
μg, μg Microgram
BID One dose twice daily
C57B1/6 Common inbred strain of laboratory mouse, also “C57 black 6”, “C57”, “black 6”, or “B6”
CDR Complementary determining region
CR Complete regression
Ctrl Control
FR Framework region
IgG Immunoglobulin G
IgG1 Immunoglobulin G subclass 1
IP Intraperitoneal
IT Intratumoral
kg Kilogram
mAb Monoclonal antibody
mg Milligram
mIgG1 Murine immunoglobulin G subclass 1, Isotype control mAb for anti-PD-1 antibody muDX400
mL Milliliter
mm Millimeter
mm³ Cubic millimeter, 0.001 mL
MPK Milligram per kilogram
n Number of subjects in a treatment group
NCI National Cancer Institute
OR Overall response
OS Overall survival
PBS Phosphate-buffered saline, vehicle control for CXCR2 antagonists
PD-1 Programmed cell death protein 1
PFS Progression free survival
PR Partial response
p-values Calculated probability
QD One dose per day
RECIST Response Evaluation Criteria in Solid Tumors
SD Stable disease
SEM Standard error of the mean
TGI Tumor growth inhibition
T/C Median tumor volume of the treated animal/Median tumor volume of the control animal
V_{H} immunoglobulin heavy chain variable region
V_{K} immunoglobulin kappa light chain variable region
V_{L} immunoglobulin light chain variable region

Additional abbreviations may be defined throughout this disclosure.

2. Definitions

Certain technical and scientific terms are specifically defined below. Unless specifically defined elsewhere in this document, all other technical and scientific terms used herein have the meaning commonly understood by one of ordinary skill in the art to which this disclosure relates.

“About” when used to modify a numerically defined parameter (e.g., the dose of an anti-PD-1 antibody or antigen binding fragment thereof, an anti-LAG3 antibody or antigen binding fragment thereof, or an anti-TIGIT antibody or antigen binding fragment thereof, or the length of treatment time with a combination therapy described herein) means that the parameter is within 20%, within 15%, within 10%, within 9%, within 8%, within 7%, within 6%, within 5%, within 4%, within 3%, within 2%, within 1%, or less of the stated numerical value or range for that parameter; where appropriate, the stated parameter may be rounded to the nearest whole number. For example, a dose of about 5 mg/kg may vary between 4.5 mg/kg and 5.5 mg/kg.

As used herein, including the appended claims, the singular forms of words such as “a,” “an,” and “the,” include their corresponding plural references unless the context clearly dictates otherwise.

The terms “administration” or “administer” refers to the act of injecting or otherwise physically delivering a substance as it exists outside the body (e.g., an anti-PD-1 antibody, an anti-LAG3 antibody, and an anti-TIGIT antibody as described herein) into a patient, such as by mucosal, intradermal, intravenous, intramuscular delivery, and/or any other methods of physical delivery described herein or known in the art.

As used herein, the term “antibody” refers to any form of immunoglobulin molecule that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, and chimeric antibodies. “Parental antibodies” are antibodies obtained by exposure of an immune system to an antigen prior to modification of the antibodies for an intended use, such as humanization of an antibody for use as a human therapeutic. As used herein, the term “antibody” encompasses not only intact polyclonal or monoclonal antibodies, but also, unless otherwise specified, any antigen binding portion thereof that competes with the intact antibody for specific binding, fusion proteins comprising an antigen binding portion, and any other modified configuration of the immunoglobulin molecule that comprises an antigen recognition site.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The variable regions of each light/heavy chain pair form the antibody binding site. Thus, in general, an intact antibody has two binding sites. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

“Variable regions” or “V region” or “V chain” as used herein means the segment of IgG chains which is variable in sequence between different antibodies. A “variable region” of an antibody refers to the variable region of the antibody light chain or the variable region of the antibody heavy chain, either alone or in combination. The variable region of the heavy chain may be referred to as “V_{H}.” The variable region of the light chain may be referred to as “V_{L}.” Typically, the variable regions of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3, and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

A “CDR” refers to one of three hypervariable regions (H1, H2, or H3) within the non-framework region of the antibody V_{H} β-sheet framework, or one of three hypervariable regions (L1, L2, or L3) within the non-framework region of the antibody V_{L} β-sheet framework. Accordingly, CDRs are variable region sequences interspersed within the framework region sequences. CDR regions are well known to those skilled in the art and have been defined by, for example, Kabat as the regions of most hypervariability within the antibody variable domains. CDR region sequences also have been defined structurally by Chothia as those residues that are not part of the conserved b-sheet framework, and thus are able to adapt to different conformation. Both terminologies are well recognized in the art. CDR region sequences have also been defined by AbM, Contact, and IMGT. The positions of CDRs within a canonical antibody variable region have been determined by comparison of numerous structures (Al-Lazikani et al., 1997, J. Mol. Biol. 273:927-48; Morea et al., 2000, Methods 20:267-79). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra). Such nomenclature is similarly well known to those skilled in the art. Correspondence between the numbering system, including, for example, the Kabat numbering and the IMGT unique numbering system, is well known to one skilled in the art and shown below in Table 1. In some embodiments, the CDRs are as defined by the Kabat numbering system. In other embodiments, the CDRs are as defined by the IMGT numbering system. In yet other embodiments, the CDRs are as defined by the AbM numbering system. In still other embodiments, the CDRs are as defined by the Chothia numbering system. In yet other embodiments, the CDRs are as defined by the Contact numbering system.

TABLE 1
Correspondence between the CDR Numbering Systems

| | Kabat + | | | | | |
|---|---|---|---|---|---|---|
| | Chothia | IMGT | Kabat | AbM | Chothia | Contact |
| V_{H} CDR1 | 26-35 | 27-38 | 31-35 | 26-35 | 26-32 | 30-35 |
| V_{H} CDR2 | 50-65 | 56-65 | 50-65 | 50-58 | 52-56 | 47-58 |
| V_{H} CDR3 | 95-102 | 105-117 | 95-102 | 95-102 | 95-102 | 93-101 |
| V_{L} CDR1 | 24-34 | 27-38 | 24-34 | 24-34 | 24-34 | 30-36 |
| V_{L} CDR2 | 50-56 | 56-65 | 50-56 | 50-56 | 50-56 | 46-55 |
| V_{L} CDR3 | 89-97 | 105-117 | 89-97 | 89-97 | 89-97 | 89-96 |

“Chimeric antibody” refers to an antibody in which a portion of the heavy and/or light chain contains sequences derived from a particular species (e.g., human) or belonging to a particular antibody class or subclass, while the remainder of the chain(s) is derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences or derivatives thereof. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences or derivatives thereof, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” may be added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al. (1975) Nature 256: 495, or may be made by recombinant DNA methods (see. e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

As used herein, unless otherwise indicated, “antibody fragment” or “antigen binding fragment” refers to a fragment of an antibody that retains the ability to bind specifically to the antigen, e.g., fragments that retain one or more CDR regions. An antibody that “specifically binds to” PD-1, LAG3, or TIGIT is an antibody that exhibits preferential binding to PD-1, LAG3, or TIGIT (as appropriate) as compared to other proteins, but this specificity does not require absolute binding specificity. An antibody is considered “specific” for its intended target if its binding is determinative of the presence of the target protein in a sample, e.g., without producing undesired results such as false positives. Antibodies, or binding fragments thereof, will bind to the target protein with an affinity that is at least two fold greater, preferably at least ten times greater, more preferably at least 20-times greater, and most preferably at least 100-times greater than the affinity with non-target proteins.

Antigen binding portions include, for example, Fab, Fab′, F(ab′)2, Fd, Fv, fragments including CDRs, and single chain variable fragment antibodies (scFv), and polypeptides that contain at least a portion of an immunoglobulin that is sufficient to confer specific antigen binding to the antigen (e.g., PD-1, LAG3, or TIGIT). An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

As used herein, the terms “at least one” item or “one or more” item each include a single item selected from the list as well as mixtures of two or more items selected from the list.

As used herein, the term “immune response” relates to any one or more of the following: specific immune response, non-specific immune response, both specific and non-specific response, innate response, primary immune response, adaptive immunity, secondary immune response, memory immune response, immune cell activation, immune cell-proliferation, immune cell differentiation, and cytokine expression.

The term “pharmaceutically acceptable carrier” refers to any inactive substance that is suitable for use in a formulation for the delivery of a therapeutic agent. A carrier may be an antiadherent, binder, coating, disintegrant, filler or diluent, preservative (such as antioxidant, antibacterial, or antifungal agent), sweetener, absorption delaying agent, wetting agent, emulsifying agent, buffer, and the like. Examples of suitable pharmaceutically acceptable carriers include water, ethanol, polyols (such as glycerol, propylene glycol, polyethylene glycol, and the like), dextrose, vegetable oils (such as olive oil), saline, buffer, buffered saline, and isotonic agents such as sugars, polyalcohols, sorbitol, and sodium chloride.

The term “subject” (alternatively “patient”) as used herein refers to a mammal that has been the object of treatment, observation, or experiment. The mammal may be male or female. The mammal may be one or more selected from the group consisting of humans, bovine (e.g., cows), porcine (e.g., pigs), ovine (e.g., sheep), capra (e.g., goats), equine (e.g., horses), canine (e.g., domestic dogs), feline (e.g., house cats), lagomorphs (e.g., rabbits), rodents (e.g., rats or mice), Procyon lotor (e.g., raccoons). In particular embodiments, the subject is human.

The term “subject in need thereof” as used herein refers to a subject diagnosed with or suspected of having cancer or an infectious disease as defined herein.

“Biotherapeutic agent” means a biological molecule, such as an antibody or fusion protein, that blocks ligand/receptor signaling in any biological pathway that supports tumor maintenance and/or growth or suppresses the anti-tumor immune response.

“Chemotherapeutic agent” refers to a chemical or biological substance that can cause death of cancer cells, or interfere with growth, division, repair, and/or function of cancer cells. Examples of chemotherapeutic agents include those that are disclosed in WO2006/129163, and US20060153808. Classes of chemotherapeutic agents include, but are not limited to: alkylating agents, antimetabolites, kinase inhibitors, spindle poison, plant alkaloids, cytoxic/antitumor antibiotics, topisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, and anti-sense oligonucleotides that inhibit expression of genes implicated in abnormal cell-proliferation or tumor growth. Chemotherapeutic agents useful in the treatment methods of the present disclosure include cytostatic and/or cytotoxic agents.

The therapeutic agents and compositions provided by the present disclosure can be administered via any suitable enteral route or parenteral route of administration. The term “enteral route” of administration refers to the administration via any part of the gastrointestinal tract. Examples of enteral routes include oral, mucosal, buccal, and rectal route, or intragastric route. “Parenteral route” of administration refers to a route of administration other than enteral route. Examples of parenteral routes of administration include intravenous, intramuscular, intradermal, intraperitoneal, intratumor, intravesical, intraarterial, intrathecal, intracapsular, intraorbital, intracardiac, transtracheal, intraarticular, subcapsular, subarachnoid, intraspinal, epidural and intrastemal, subcutaneous, or topical administration. The therapeutic agents and compositions of the disclosure can be administered using any suitable method, such as by oral ingestion, nasogastric tube, gastrostomy tube, injection, infusion, implantable infusion pump, and osmotic pump. The suitable route and method of administration may vary depending on a number of factors such as the specific therapeutic agent being used, the rate of absorption desired, specific formulation or dosage form used, type or severity of the disorder being treated, the specific site of action, and conditions of the patient, and can be readily selected by a person skilled in the art.

The term “variant” when used in relation to an antibody (e.g., an anti-PD-1 antibody, an anti-LAG3 antibody, or an anti-TIGIT antibody) or an amino acid region within the antibody may refer to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence. For example, a variant of an anti-PD-1 antibody may result from one or more (such as, for example, about 1 to about 25, about 1 to about 20, about 1 to about 15, about 1 to about 10, or about 1 to about 5) changes to an amino acid sequence of a native or previously unmodified anti-PD-1 antibody. Variants may be naturally occurring or may be artificially constructed. Polypeptide variants may be prepared from the corresponding nucleic acid molecules encoding the variants. In specific embodiments, an antibody variant (e.g., an anti-PD-1 antibody variant, an anti-LAG3 antibody variant, or an anti-TIGIT antibody variant) at least retains the antibody functional activity. In specific embodiments, an anti-PD-1 antibody variant binds to PD-1 and/or is antagonistic to PD-1 activity. In some embodiments, an anti-LAG3 antibody variant binds to LAG3 and/or is antagonistic to LAG3 activity. In some embodiments, an anti-TIGIT antibody variant binds to TIGIT and/or is antagonistic to TIGIT activity.

“Conservatively modified variants” or “conservative substitution” refers to substitutions of amino acids in a protein with other amino acids having similar characteristics (e.g., charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see. e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 2 below.

TABLE 2
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition.

“Homology” refers to sequence similarity between two polypeptide sequences when they are optimally aligned. When a position in both of the two compared sequences is occupied by the same amino acid monomer subunit, e.g., if a position in a light chain CDR of two different Abs is occupied by alanine, then the two Abs are homologous at that position. The percent of homology is the number of homologous positions shared by the two sequences divided by the total number of positions compared×100. For example, if 8 of 10 of the positions in two sequences are matched when the sequences are optimally aligned then the two sequences are 80% homologous. Generally, the comparison is made when two sequences are aligned to give maximum percent homology. For example, the comparison can be performed by a BLAST algorithm wherein the parameters of the algorithm are selected to give the largest match between the respective sequences over the entire length of the respective reference sequences.

The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Nail. Biomed. Res. Found., Washington, DC; Schwartz, R. M., et al., “Matrices for detecting distant relationships.” in Atlas of Protein Sequence and Structure, (1978) vol. 5, suppl. 3.″ M. O. Dayhoff (ed.), pp. 353-358, Natl. Biomed. Res. Found., Washington, DC; Altschul, S. F., (1991) J. Mol. Biol. 219:555-565; States, D. J., et al., (1991) Methods 3:66-70; Henikoff, S., et al., (1992) Proc. Nail. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

“RECIST 1.1 Response Criteria” as used herein means the definitions set forth in Eisenhauer, E. A. et al., Eur. J. Cancer 45:228-247 (2009) for target lesions or nontarget lesions, as appropriate based on the context in which response is being measured.

“Sustained response” means a sustained therapeutic effect after cessation of treatment as described herein. In some embodiments, the sustained response has a duration that is at least the same as the treatment duration, or at least 1.5, 2.0, 2.5 or 3 times longer than the treatment duration.

“Treat” or “treating” cancer as used herein means to administer a therapeutic combination of an anti-human PD-1 monoclonal antibody or antigen binding fragment thereof, an anti-human LAG3 monoclonal antibody or antigen binding fragment thereof, and an anti-human TIGIT monoclonal antibody or antigen binding fragment thereof, to a subject having cancer or diagnosed with cancer to achieve at least one positive therapeutic effect, such as, for example, reduced number of cancer cells, reduced tumor size, reduced rate of cancer cell infiltration into peripheral organs, or reduced rate of tumor metastasis or tumor growth. Such “treatment” may result in a slowing, interrupting, arresting, controlling, or stopping of the progression of cancer as described herein but does not necessarily indicate a total elimination of the cancer or the symptoms of the cancer. Positive therapeutic effects in cancer can be measured in a number of ways (See, W. A. Weber, J. Nucl. Med. 50:1S-10S (2009)). For example, with respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. In some embodiments, the treatment achieved by a combination therapy of the disclosure is any of PR, CR, OR, PFS, DFS, and OS. PFS, also referred to as “Time to Tumor Progression” indicates the length of time during and after treatment that the cancer does not grow, and includes the amount of time patients have experienced a CR or PR, as well as the amount of time patients have experienced SD. DFS refers to the length of time during and after treatment that the patient remains free of disease. OS refers to a prolongation in life expectancy as compared to naive or untreated individuals or patients. In some embodiments, response to a combination therapy of the disclosure is any of PR, CR, PFS, DFS, or OR that is assessed using RECIST 1.1 response criteria. The treatment regimen for a combination therapy of the disclosure that is effective to treat a cancer patient may vary according to factors such as the disease state, age, and weight of the patient, and the ability of the therapy to elicit an anti-cancer response in the subject. While an embodiment of any of the aspects of the disclosure may not be effective in achieving a positive therapeutic effect in every subject, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

As used herein, the terms “combination therapy” and “therapeutic combination” refer to treatments in which at least one anti-human PD-1 monoclonal antibody or antigen-binding fragment thereof, at least one anti-human LAG3 monoclonal antibody or antigen-binding fragment thereof, and at least one anti-human TIGIT monoclonal antibody or antigen-binding fragment thereof, and optionally additional therapeutic agents, each are administered to a patient in a coordinated manner, over an overlapping period of time. The period of treatment with the at least one anti-human PD-1 monoclonal antibody (or antigen-binding fragment thereof) (the “anti-PD-1 treatment”) is the period of time that a patient undergoes treatment with the anti-human PD-1 monoclonal antibody (or antigen-binding fragment thereof); that is, the period of time from the initial dosing with the anti-human PD-1 monoclonal antibody (or antigen-binding fragment thereof) through the final day of a treatment cycle. Similarly, the period of treatment with the at least one anti-human LAG3 monoclonal antibody (or antigen-binding fragment thereof) (the “anti-LAG3 treatment”) is the period of time that a patient undergoes treatment with the anti-human LAG3 monoclonal antibody (or antigen-binding fragment thereof); that is, the period of time from the initial dosing with the anti-human LAG3 monoclonal antibody (or antigen-binding fragment thereof) through the final day of a treatment cycle. The period of treatment with the at least one anti-human TIGIT monoclonal antibody (or antigen-binding fragment thereof) (the “anti-TIGIT treatment”) is the period of time that a patient undergoes treatment with the anti-human TIGIT monoclonal antibody (or antigen-binding fragment thereof); that is, the period of time from the initial dosing with the anti-human TIGIT monoclonal antibody (or antigen-binding fragment thereof) through the final day of a treatment cycle. In the methods and therapeutic combinations described herein, the anti-PD-1 treatment overlaps by at least one day with the anti-LAG3 treatment and overlaps by at least one day with the anti-TIGIT treatment. In certain embodiments, the anti-PD-1 treatment, the anti-LAG3 treatment, and the anti-TIGIT treatment are the same period of time. In some embodiments, the anti-PD-1 treatment begins prior to the anti-LAG3 and/or the anti-TIGIT treatment. In other embodiments, the anti-PD-1 treatment begins after the anti-LAG3 and/or the anti-TIGIT treatment. In yet other embodiments, the anti-LAG3 treatment begins prior to the anti-PD-1 and/or the anti-TIGIT treatment. In still other embodiments, the anti-LAG3 treatment begins after the anti-PD-1 and/or the anti-TIGIT treatment. In some embodiments, the anti-TIGIT treatment begins prior to the anti-LAG3 and/or the anti-PD-1 treatment. In other embodiments, the anti-TIGIT treatment begins after the anti-LAG3 and/or the anti-PD-1 treatment. In certain embodiments, the anti-PD-1 treatment is terminated prior to termination of the anti-LAG3 and/or the anti-TIGIT treatment. In other embodiments, the anti-PD-1 treatment is terminated after termination of the anti-LAG3 and/or the anti-TIGIT treatment. In yet other embodiments, the anti-LAG3 treatment is terminated prior to termination of the anti-PD-1 and/or the anti-TIGIT treatment. In still other embodiments, the anti-LAG3 treatment is terminated after termination of the anti-PD-1 and/or the anti-TIGIT treatment. In certain embodiments, the anti-TIGIT treatment is terminated prior to termination of the anti-LAG3 and/or the anti-PD-1 treatment. In other embodiments, the anti-TIGIT treatment is terminated after termination of the anti-LAG3 and/or the anti-PD-1 treatment.

The terms “treatment regimen,” “dosing protocol,” and “dosing regimen” are used interchangeably to refer to the dose and timing of administration of each therapeutic agent in a combination therapy of the disclosure.

“Tumor” as it applies to a subject diagnosed with, or suspected of having, a cancer refers to a malignant or potentially malignant neoplasm or tissue mass of any size, and includes primary tumors and secondary neoplasms. Non-limiting examples of tumors include solid tumor (e.g., sarcoma (such as chondrosarcoma), carcinoma (such as colon carcinoma), blastoma (such as hepatoblastoma), etc.) and blood tumor (e.g., leukemia (such as acute myeloid leukemia (AML)), lymphoma (such as DLBCL), multiple myeloma (MM), etc.).

“Tumor burden” also referred to as “tumor load”, refers to the total amount of tumor material distributed throughout the body. Tumor burden refers to the total number of cancer cells or the total size of tumor(s), throughout the body, including lymph nodes and bone narrow. Tumor burden can be determined by a variety of methods known in the art, such as, e.g., by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor volume” or “tumor size” refers to the total size of the tumor which can be measured as the length and width of a tumor. Tumor size may be determined by a variety of methods known in the art, such as, e.g., by measuring the dimensions of tumor(s) upon removal from the subject, e.g., using calipers, or while in the body using imaging techniques, e.g., bone scan, ultrasound, CT or MRI scans.

It is understood that wherever embodiments are described herein with the language “comprising,” otherwise analogous embodiments described in terms of “consisting of” and/or “consisting essentially of” are also provided.

Unless expressly stated to the contrary, all ranges cited herein are inclusive; i.e., the range includes the values for the upper and lower limits of the range as well as all values in between. As an example, temperature ranges, percentages, ranges of equivalents, and the like described herein include the upper and lower limits of the range and any value in the continuum there between. Numerical values provided herein, and the use of the term “about”, may include variations of ±1%, ±2%, ±3%, ±4%, ±5%, ±10%, ±15%, and ±20% and their numerical equivalents. All ranges also are intended to include all included sub-ranges, although not necessarily explicitly set forth. For example, a range of 3 to 7 days is intended to include 3, 4, 5, 6, and 7 days. In addition, the term “or,” as used herein, denotes alternatives that may, where appropriate, be combined; that is, the term “or” includes each listed alternative separately as well as their combination.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in the claims.

Unless otherwise defined, all technical and scientific terms used herein have the same meaning as commonly understood by one of ordinary skill in the art to which this disclosure relates. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting.

Exemplary methods and materials are described herein, although methods and materials similar or equivalent to those described herein can also be used in the practice or testing of the present disclosure. The materials, methods, and examples are illustrative only and not intended to be limiting.

3. Anti-human PD-1 Monoclonal Antibodies and Antigen Binding Fragments Thereof

Provided herein are anti-human PD-1 monoclonal antibodies or antigen binding fragments thereof that can be used in the various methods, pharmaceutical compositions, kits, and uses disclosed herein. Any monoclonal antibodies that bind to a PD-1 polypeptide, a PD-1 polypeptide fragment, a PD-1 peptide, or a PD-1 epitope and block the interaction between PD-1 and its ligand PD-L1 or PD-L2 can be used. In some embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L1. In other embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L2. In yet other embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L1 and the interaction between PD-1 and PD-L2.

In certain embodiments of various methods, pharmaceutical compositions, kits, or uses provided herein, the anti-human PD-1 monoclonal antibody or antigen binding fragment thereof comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively.

In some embodiments of various methods, pharmaceutical compositions, kits, or uses provided herein, the anti-human PD-1 monoclonal antibody or antigen binding fragment thereof comprises a V_{L} region comprising an amino acid sequence as set forth in SEQ ID NO:4, and a V_{H} region comprising an amino acid sequence as set forth in SEQ ID NO:9.

In other embodiments of various methods, pharmaceutical compositions, kits, or uses provided herein, the anti-human PD-1 monoclonal antibody or antigen binding fragment thereof comprises a light chain comprising or consisting of an amino acid sequence as set forth in SEQ ID NO:5 and a heavy chain comprising or consisting of an amino acid sequence as set forth in SEQ ID NO:10.

In certain embodiments of various methods, pharmaceutical compositions, kits, or uses provided herein, the anti-human PD-1 monoclonal antibody or antigen binding fragment thereof comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively.

In some embodiments of various methods, pharmaceutical compositions, kits, or uses provided herein, the anti-human PD-1 monoclonal antibody or antigen binding fragment thereof comprises a V_{L} region comprising an amino acid sequence as set forth in SEQ ID NO:34, and a V_{H} region comprising an amino acid sequence as set forth in SEQ ID NO:39.

In other embodiments of various methods, pharmaceutical compositions, kits, or uses provided herein, the anti-human PD-1 monoclonal antibody or antigen binding fragment thereof comprises a light chain comprising or consisting of an amino acid sequence as set forth in SEQ ID NO:35 and a heavy chain comprising or consisting of an amino acid sequence as set forth in SEQ ID NO:40.

In various embodiments, the anti-human PD-1 monoclonal antibody or antigen binding fragment thereof comprises a variant of the amino acid sequences disclosed herein. A variant amino acid sequence is identical to the reference sequence except having one, two, three, four, or five amino acid substitutions, deletions, and/or additions. In some embodiments, the substitutions, deletions and/or additions are in the CDRs. In some embodiments, the substitutions, deletions and/or additions are in the framework regions. In certain embodiments, the one, two, three, four, or five of the amino acid substitutions are conservative substitutions.

In one embodiment, the anti-human PD-1 monoclonal antibody or antigen binding fragment thereof has a V_{L} domain with at least 95%, 90%, 85%, 80%, 75% or 50% sequence homology to one of the V_{L} domains described herein, and exhibits specific binding to PD-1. In another embodiment, the anti-human PD-1 monoclonal antibody or antigen binding fragment thereof has a V_{H} domain with at least 95%, 90%, 85%, 80%, 75% or 50% sequence homology to one of the V_{H} domains described herein, and exhibits specific binding to PD-1. In yet another embodiment, the anti-human PD-1 monoclonal antibody or antigen binding fragment thereof has a V_{L} domain with at least 95%, 90%, 85%, 80%, 75% or 50% sequence homology to one of the V_{L} domains described herein and a V_{H} domain with at least 95%, 90%, 85%, 80%, 75% or 50% sequence homology to one of the V_{H} domains described herein, and exhibits specific binding to PD-1.

In one embodiment, the anti-human PD-1 monoclonal antibody or antigen binding fragment thereof has a V_{L} domain having up to 1, 2, 3, 4, 5 or more amino acid substitutions, deletions and/or additions in one of the V_{L} domains described herein, and exhibits specific binding to PD-1. In another embodiment, the anti-human PD-1 monoclonal antibody or antigen binding fragment thereof has a V_{H} domain having up to 1, 2, 3, 4, 5 or more amino acid substitutions, deletions, and/or additions in one of the V_{H} domains described herein, and exhibits specific binding to PD-1. In yet another embodiment, the anti-human PD-1 monoclonal antibody or antigen binding fragment thereof has a V_{L} domain having up to 1, 2, 3, 4, 5 or more amino acid substitutions, deletions, and/or additions in one of the V_{L} domains described herein and a V_{H} domain having up to 1, 2, 3, 4, 5 or more amino acid substitutions, deletions, and/or additions in one of the V_{H} domains described herein, and exhibits specific binding to PD-1.

In various embodiments, the anti-human PD-1 monoclonal antibody or antigen binding fragment thereof is selected from any class of immunoglobulins, including IgM, IgG, IgD, IgA, and IgE. Preferably, the antibody is an IgG antibody. Any isotype of IgG can be used, including IgG₁, IgG₂, IgG₃, and IgG₄. Different constant domains may be appended to the V_{L} and V_{H} regions provided herein. For example, if a particular intended use of an antibody (or fragment) of the present invention were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances, an IgG4 constant domain, for example, may be used. In various embodiments, the heavy chain constant domain contains one or more amino acid mutations (e.g., IgG4 with S228P mutation) to generate desired characteristics of the antibody. These desired characteristics include but are not limited to modified effector functions, physical or chemical stability, half-life of antibody, etc.

Ordinarily, amino acid sequence variants of the anti-PD-1 monoclonal antibodies and antigen binding fragments thereof disclosed herein will have an amino acid sequence having at least 75% amino acid sequence identity with the amino acid sequence of a reference antibody or antigen binding fragment (e.g., heavy chain, light chain, V_{H}, V_{L}, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the reference sequence, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3. M. O. Dayhoff(ed.), pp. 353-358, Natl. Biomed. Res. Found., Washington, DC; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

In some embodiments, the anti-human PD-1 monoclonal antibody is a human antibody. In other embodiments, the anti-human PD-1 monoclonal antibody is a humanized antibody.

In some embodiments, the heavy chain of the anti-human PD-1 monoclonal antibody has a human IgG1 backbone. In other embodiments, the heavy chain of the anti-human PD-1 monoclonal antibody has a human IgG2 backbone. In yet other embodiments, the heavy chain of the anti-human PD-1 monoclonal antibody has a human IgG3 backbone. In still other embodiments, the heavy chain of the anti-human PD-1 monoclonal antibody has a human IgG4 backbone.

In some embodiments, the heavy chain of the anti-human PD-1 monoclonal antibody has a human IgG1 variant backbone. In other embodiments, the heavy chain of the anti-human PD-1 monoclonal antibody has a human IgG2 variant backbone. In yet other embodiments, the heavy chain of the anti-human PD-1 monoclonal antibody has a human IgG3 variant backbone. In still other embodiments, the heavy chain of the anti-human PD-1 monoclonal antibody has a human IgG4 variant (e.g., IgG4 with S228P mutation) backbone.

In certain embodiments, the anti-human PD-1 monoclonal antibody is selected from the group consisting of pembrolizumab, nivolumab, cemiplimab, pidilizumab, AMP-514, PDR001, BGB-A317, and MGA012. In one embodiment, the anti-human PD-1 monoclonal antibody is pembrolizumab. In another embodiment, the anti-human PD-1 monoclonal antibody is nivolumab. In another embodiment, the anti-human PD-1 monoclonal antibody is cemiplimab. In yet another embodiment, the anti-human PD-1 monoclonal antibody is pidilizumab. In one embodiment, the anti-human PD-1 monoclonal antibody is AMP-514. In another embodiment, the anti-human PD-1 monoclonal antibody is PDR001. In yet another embodiment, the anti-human PD-1 monoclonal antibody is BGB-A317. In still another embodiment, the anti-human PD-1 monoclonal antibody is MGA012.

In some embodiments, the anti-human PD-1 monoclonal antibody can be any antibody, antigen binding fragment thereof, or variant thereof disclosed in WO 2008/156712, the disclosure of which is incorporated by reference herein in its entirety.

4. Anti-human LAG3 Monoclonal Antibodies and Antigen Binding Fragments Thereof

Also provided herein are anti-human LAG3 monoclonal antibodies or antigen binding fragments thereof that can be used in the various methods, pharmaceutical compositions, kits, and uses disclosed herein. Any monoclonal antibodies that bind to a LAG3 polypeptide, a LAG3 polypeptide fragment, a LAG3 peptide, or a LAG3 epitope and block the interaction between LAG3 and its ligand Class II MHC can be used.

In certain embodiments of various methods, pharmaceutical compositions, kits, or uses provided herein, the anti-human LAG3 monoclonal antibody or antigen binding fragment thereof comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively.

In some embodiments of various methods, pharmaceutical compositions, kits, or uses provided herein, the anti-human LAG3 monoclonal antibody or antigen binding fragment thereof comprises a V_{L} region comprising an amino acid sequence as set forth in SEQ ID NO:14, and a V_{H} region comprising an amino acid sequence as set forth in SEQ ID NO:19.

In other embodiments of various methods, pharmaceutical compositions, kits, or uses provided herein, the anti-human LAG3 monoclonal antibody or antigen binding fragment thereof comprises a light chain comprising or consisting of an amino acid sequence as set forth in SEQ ID NO:15 and a heavy chain comprising or consisting of an amino acid sequence as set forth in SEQ ID NO:20.

In certain embodiments of various methods, pharmaceutical compositions, kits, or uses provided herein, the anti-human LAG3 monoclonal antibody or antigen binding fragment thereof comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 41, and 18, respectively.

In certain embodiments of various methods, pharmaceutical compositions, kits, or uses provided herein, the anti-human LAG3 monoclonal antibody or antigen binding fragment thereof comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 42, and 18, respectively.

In certain embodiments of various methods, pharmaceutical compositions, kits, or uses provided herein, the anti-human LAG3 monoclonal antibody or antigen binding fragment thereof comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 43, and 18, respectively.

In certain embodiments of various methods, pharmaceutical compositions, kits, or uses provided herein, the anti-human LAG3 monoclonal antibody or antigen binding fragment thereof comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 44, and 18, respectively.

In some embodiments of various methods, pharmaceutical compositions, kits, or uses provided herein, the anti-human LAG3 monoclonal antibody or antigen binding fragment thereof comprises a V_{L} region comprising an amino acid sequence as set forth in SEQ ID NO:14, and a V_{H} region comprising an amino acid sequence as set forth in SEQ ID NO:45.

In some embodiments of various methods, pharmaceutical compositions, kits, or uses provided herein, the anti-human LAG3 monoclonal antibody or antigen binding fragment thereof comprises a V_{L} region comprising an amino acid sequence as set forth in SEQ ID NO:14, and a V_{H} region comprising an amino acid sequence as set forth in SEQ ID NO:46.

In some embodiments of various methods, pharmaceutical compositions, kits, or uses provided herein, the anti-human LAG3 monoclonal antibody or antigen binding fragment thereof comprises a V_{L} region comprising an amino acid sequence as set forth in SEQ ID NO:14, and a V_{H} region comprising an amino acid sequence as set forth in SEQ ID NO:47.

In some embodiments of various methods, pharmaceutical compositions, kits, or uses provided herein, the anti-human LAG3 monoclonal antibody or antigen binding fragment thereof comprises a V_{L} region comprising an amino acid sequence as set forth in SEQ ID NO:14, and a V_{H} region comprising an amino acid sequence as set forth in SEQ ID NO:48.

In other embodiments of various methods, pharmaceutical compositions, kits, or uses provided herein, the anti-human LAG3 monoclonal antibody or antigen binding fragment thereof comprises a light chain comprising or consisting of an amino acid sequence as set forth in SEQ ID NO:15 and a heavy chain comprising or consisting of an amino acid sequence as set orth in SEQ ID NO:49.

In other embodiments of various methods, pharmaceutical compositions, kits, or uses provided herein, the anti-human LAG3 monoclonal antibody or antigen binding fragment thereof comprises a light chain comprising or consisting of an amino acid sequence as set forth in SEQ ID NO:15 and a heavy chain comprising or consisting of an amino acid sequence as set forth in SEQ ID NO:50.

In other embodiments of various methods, pharmaceutical compositions, kits, or uses provided herein, the anti-human LAG3 monoclonal antibody or antigen binding fragment thereof comprises a light chain comprising or consisting of an amino acid sequence as set forth in SEQ ID NO:15 and a heavy chain comprising or consisting of an amino acid sequence as set forth in SEQ ID NO:51.

In other embodiments of various methods, pharmaceutical compositions, kits, or uses provided herein, the anti-human LAG3 monoclonal antibody or antigen binding fragment thereof comprises a light chain comprising or consisting of an amino acid sequence as set forth in SEQ ID NO:15 and a heavy chain comprising or consisting of an amino acid sequence as set forth in SEQ ID NO:52.

In other embodiments of various methods, pharmaceutical compositions, kits, or uses provided herein, the anti-human LAG3 monoclonal antibody or antigen binding fragment thereof comprises a light chain comprising or consisting of an amino acid sequence as set forth in SEQ ID NO:15 and a heavy chain comprising or consisting of an amino acid sequence as set forth in SEQ ID NO:53.

In other embodiments of various methods, pharmaceutical compositions, kits, or uses provided herein, the anti-human LAG3 monoclonal antibody or antigen binding fragment thereof comprises a light chain comprising or consisting of an amino acid sequence as set forth in SEQ ID NO:15 and a heavy chain comprising or consisting of an amino acid sequence as set forth in SEQ ID NO:54.

In other embodiments of various methods, pharmaceutical compositions, kits, or uses provided herein, the anti-human LAG3 monoclonal antibody or antigen binding fragment thereof comprises a light chain comprising or consisting of an amino acid sequence as set forth in SEQ ID NO:15 and a heavy chain comprising or consisting of an amino acid sequence as set forth in SEQ ID NO:55.

In other embodiments of various methods, pharmaceutical compositions, kits, or uses provided herein, the anti-human LAG3 monoclonal antibody or antigen binding fragment thereof comprises a light chain comprising or consisting of an amino acid sequence as set forth in SEQ ID NO:15 and a heavy chain comprising or consisting of an amino acid sequence as set forth in SEQ ID NO:56.

In various embodiments, the anti-human LAG3 monoclonal antibody or antigen binding fragment thereof comprises a variant of the amino acid sequences disclosed herein. A variant amino acid sequence is identical to the reference sequence except having one, two, three, four, or five amino acid substitutions, deletions, and/or additions. In some embodiments, the substitutions, deletions and/or additions are in the CDRs. In some embodiments, the substitutions, deletions and/or additions are in the framework regions. In certain embodiments, the one, two, three, four, or five of the amino acid substitutions are conservative substitutions.

In one embodiment, the anti-human LAG3 monoclonal antibody or antigen binding fragment thereof has a V_{L} domain with at least 95%, 90%, 85%, 80%, 75% or 50% sequence homology to one of the V_{L} domains described herein, and exhibits specific binding to LAG3. In another embodiment, the anti-human LAG3 monoclonal antibody or antigen binding fragment thereof has a V_{H} domain with at least 95%, 90%, 85%, 80%, 75% or 50% sequence homology to one of the V_{H} domains described herein, and exhibits specific binding to LAG3. In yet another embodiment, the anti-human LAG3 monoclonal antibody or antigen binding fragment thereof has a V_{L} domain with at least 95%, 90%, 85%, 80%, 75% or 50% sequence homology to one of the V_{L} domains described herein and a V_{H} domain with at least 95%, 90%, 85%, 80%, 75% or 50% sequence homology to one of the V_{H} domains described herein, and exhibits specific binding to LAG3.

In one embodiment, the anti-human LAG3 monoclonal antibody or antigen binding fragment thereof has a V_{L} domain having up to 1, 2, 3, 4, 5 or more amino acid substitutions, deletions and/or additions in one of the V_{L} domains described herein, and exhibits specific binding to LAG3. In another embodiment, the anti-human LAG3 monoclonal antibody or antigen binding fragment thereof has a V_{H} domain having up to 1, 2, 3, 4, 5 or more amino acid substitutions, deletions, and/or additions in one of the V_{H} domains described herein, and exhibits specific binding to LAG3. In yet another embodiment, the anti-human LAG3 monoclonal antibody or antigen binding fragment thereof has a V_{L} domain having up to 1, 2, 3, 4, 5 or more amino acid substitutions, deletions, and/or additions in one of the V_{L} domains described herein and a V_{H} domain having up to 1, 2, 3, 4, 5 or more amino acid substitutions, deletions, and/or additions in one of the V_{H} domains described herein, and exhibits specific binding to LAG3.

In various embodiments, the anti-human LAG3 monoclonal antibody or antigen binding fragment thereof is selected from any class of immunoglobulins, including IgM, IgG, IgD, IgA, and IgE. Preferably, the antibody is an IgG antibody. Any isotype of IgG can be used, including IgG₁, IgG₂, IgG₃, and IgG₄. Different constant domains may be appended to the V_{L} and V_{H} regions provided herein. For example, if a particular intended use of an antibody (or fragment) of the present invention were to call for altered effector functions, a heavy chain constant domain other than IgG₁ may be used. Although IgG₁ antibodies provide for long half-life and for effector functions, such as complement activation and antibody-dependent cellular cytotoxicity, such activities may not be desirable for all uses of the antibody. In such instances, an IgG₄ constant domain, for example, may be used. In various embodiments, the heavy chain constant domain contains one or more amino acid mutations (e.g., IgG₄ with S228P mutation) to generate desired characteristics of the antibody. These desired characteristics include but are not limited to modified effector functions, physical or chemical stability, half-life of antibody, etc.

Ordinarily, amino acid sequence variants of the anti-LAG3 monoclonal antibodies and antigen binding fragments thereof disclosed herein will have an amino acid sequence having at least 75% amino acid sequence identity with the amino acid sequence of a reference antibody or antigen binding fragment (e.g., heavy chain, light chain, V_{H}, V_{L}, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the reference sequence, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3. M. O. Dayhoff(ed.), pp. 353-358, Natl. Biomed. Res. Found., Washington, DC; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

In some embodiments, the anti-human LAG3 monoclonal antibody is a human antibody. In other embodiments, the anti-human LAG3 monoclonal antibody is a humanized antibody.

In some embodiments, the heavy chain of the anti-human LAG3 monoclonal antibody has a human IgG1 backbone. In other embodiments, the heavy chain of the anti-human LAG3 monoclonal antibody has a human IgG2 backbone. In yet other embodiments, the heavy chain of the anti-human LAG3 monoclonal antibody has a human IgG3 backbone. In still other embodiments, the heavy chain of the anti-human LAG3 monoclonal antibody has a human IgG4 backbone.

In some embodiments, the heavy chain of the anti-human LAG3 monoclonal antibody has a human IgG1 variant backbone. In other embodiments, the heavy chain of the anti-human LAG3 monoclonal antibody has a human IgG2 variant backbone. In yet other embodiments, the heavy chain of the anti-human LAG3 monoclonal antibody has a human IgG3 variant backbone. In still other embodiments, the heavy chain of the anti-human LAG3 monoclonal antibody has a human IgG4 variant (e.g., IgG4 with S228P mutation) backbone.

In certain embodiments, the anti-human LAG3 monoclonal antibody is selected from the group consisting of BMS-986016, REGN3767, LAG525, and GSK2813781. In one embodiment, the anti-human LAG3 monoclonal antibody is BMS-986016. In another embodiment, the anti-human LAG3 monoclonal antibody is REGN3767. In yet another embodiment, the anti-human LAG3 monoclonal antibody is LAG525. In still another embodiment, the anti-human LAG3 monoclonal antibody is GSK2813781.

In some embodiments, the anti-human LAG3 monoclonal antibody can be any antibody, antigen binding fragment thereof, or variant thereof disclosed in WO 2016/028672, the disclosure of which is incorporated by reference herein in its entirety.

5. Anti-human TIGIT Monoclonal Antibodies and Antigen Binding Fragments Thereof

In addition, provided herein are anti-human TIGIT monoclonal antibodies or antigen binding fragments thereof that can be used in the various methods, pharmaceutical compositions, kits, and uses disclosed herein. Any monoclonal antibodies that bind to a TIGIT polypeptide, a TIGIT polypeptide fragment, a TIGIT peptide, or a TIGIT epitope and block the interaction between TIGIT and its ligand CD155 and/or CD112 can be used. In some embodiments, the anti-human TIGIT monoclonal antibody binds to a TIGIT polypeptide, a TIGIT polypeptide fragment, a TIGIT peptide, or a TIGIT epitope and blocks the interaction between TIGIT and CD155. In other embodiments, the anti-human TIGIT monoclonal antibody binds to a TIGIT polypeptide, a TIGIT polypeptide fragment, a TIGIT peptide, or a TIGIT epitope and blocks the interaction between TIGIT and CD112. In yet other embodiments, the anti-human TIGIT monoclonal antibody binds to a TIGIT polypeptide, a TIGIT polypeptide fragment, a TIGIT peptide, or a TIGIT epitope and blocks the interaction between TIGIT and CD155 and the interaction between TIGIT and CD112.

In certain embodiments of various methods, pharmaceutical compositions, kits, or uses provided herein, the anti-human TIGIT monoclonal antibody or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In some embodiments of various methods, pharmaceutical compositions, kits, or uses provided herein, the anti-human TIGIT monoclonal antibody or antigen binding fragment thereof comprises a V_{L} region comprising an amino acid sequence as set forth in SEQ ID NO:24, and a V_{H} region comprising an amino acid sequence as set forth in SEQ ID NO:29.

In other embodiments of various methods, pharmaceutical compositions, kits, or uses provided herein, the anti-human TIGIT monoclonal antibody or antigen binding fragment thereof comprises a light chain comprising or consisting of an amino acid sequence as set forth in SEQ ID NO:25 and a heavy chain comprising or consisting of an amino acid sequence as set forth in SEQ ID NO:30.

In certain embodiments of various methods, pharmaceutical compositions, kits, or uses provided herein, the anti-human TIGIT monoclonal antibody or antigen binding fragment thereof comprises a V_{L} CDR1 comprising the amino acid sequence as set forth in SEQ ID NO:21, a V_{L} CDR2 comprising the amino acid sequence as set forth in one of SEQ ID NOS:22 or 92-104, a V_{L} CDR3 comprising the amino acid sequence as set forth in SEQ ID NO:23, and a V_{H} CDR1 comprising the amino acid sequence as set forth in SEQ ID NO:26, a V_{H} CDR2 comprising the amino acid sequence as set forth in one of SEQ ID NOS:27 or 58-76, a V_{H} CDR3 comprising the amino acid sequence as set forth in one of SEQ ID NOS:28 or 77-91.

In certain embodiments of various methods, pharmaceutical compositions, kits, or uses provided herein, the anti-human TIGIT monoclonal antibody or antigen binding fragment thereof comprises a V_{L} CDR1 comprising the amino acid sequence as set forth in SEQ ID NO:21; a V_{L} CDR2 comprising the amino acid sequence as set forth in SEQ ID NO:104 (X₁X₂KTLAE), wherein X₁ is N, A, V, W, S, T, R, H, G, I, or V, and wherein X₂ is A, N, I, L, T, or V; a V_{L} CDR3 comprising the amino acid sequence as set forth in SEQ ID NO:23; a V_{H} CDR1 comprising the amino acid sequence as set forth in SEQ ID NO:26; a V_{H} CDR2 comprising the amino acid sequence as set forth in SEQ ID NO:76 (YIDPYNX₇X₈AKYX₁₂X₁₃KFX₁₆G), wherein X₇ is D, R, L, K, F, S, Y or V, wherein Xs is G, R, N, Q, E, L K, S, Y or V, wherein X₁₂ is N, A or S, wherein X₁₃ is E or Q, and wherein X₁₆ is K or Q; and a V_{H} CDR3 comprising the amino acid sequence as set forth in SEQ ID NO:91 (GGPYGX₆YFDV), wherein X₆ is W, A, D, E, F, G, I, K, N, Q, R, S, T, V or Y.

In certain embodiments of various methods, pharmaceutical compositions, kits, or uses provided herein, the anti-human TIGIT monoclonal antibody or antigen binding fragment thereof comprises a V_{L} that comprises a V_{L} CDR1, a V_{L} CDR2, and a V_{L} CDR3, and a V_{H} that comprises a V_{H} CDR1, a V_{H} CDR2, and a V_{H} CDR3; the V_{L} CDR1 comprising the amino acid sequence as set forth in SEQ ID NO:21; the V_{L} CDR2 comprising the amino acid sequence as set forth in one of SEQ ID NOS:22 or 92-104; the V_{L} CDR3 comprising the amino acid sequence as set forth in SEQ ID NO:23; the V_{H} CDR1 comprising the amino acid sequence as set forth in SEQ ID NO:26; the V_{H} CDR2 comprising the amino acid sequence as set forth in one of SEQ ID NOS:27 or 58-76; the V_{H} CDR3 comprising the amino acid sequence as set forth in one of SEQ ID NOS:28 or 77-91.

In certain embodiments of various methods, pharmaceutical compositions, kits, or uses provided herein, the anti-human TIGIT monoclonal antibody or antigen binding fragment thereof comprises a V_{L} that comprises a V_{L} CDR1, a V_{L} CDR2, and a V_{L} CDR3, and a V_{H} that comprises a V_{H} CDR1, a V_{H} CDR2, and a V_{H} CDR3; the V_{L} CDR1 comprising the amino acid sequence as set forth in SEQ ID NO:21; the V_{L} CDR2 comprising the amino acid sequence as set forth in SEQ ID NO:104 (X₁X₂KTLAE), wherein X₁ is N, A, V, W, S, T, R, H, G, I, or V, and wherein X₂ is A, N, I, L, T, or V; the V_{L} CDR3 comprising the amino acid sequence as set forth in SEQ ID NO:23; the V_{H} CDR1 comprising the amino acid sequence as set forth in SEQ ID NO:26; the V_{H} CDR2 comprising the amino acid sequence as set forth in SEQ ID NO:76 (YIDPYNX₇X₈AKYX₁₂X₁₃KFX₁₆G), wherein X₇ is D, R, L, K, F, S, Y or V, wherein X₈ is G, R, N, Q, E, L K, S, Y or V, wherein X₁₂ is N, A or S, wherein X₁₃ is E or Q, and wherein X₁₆ is K or Q; and the V_{H} CDR3 comprising the amino acid sequence as set forth in SEQ ID NO:91 (GGPYGX₆YFDV), wherein X₆ is W, A, D, E, F, G, I, K, N, Q, R, S, T, V or Y.

In certain embodiments of various methods, pharmaceutical compositions, kits, or uses provided herein, the anti-human TIGIT monoclonal antibody or antigen binding fragment thereof comprises a light chain that comprises a V_{L} CDR1, a V_{L} CDR2, and a V_{L} CDR3, and a heavy chain that comprises a V_{H} CDR1, a V_{H} CDR2, and a V_{H} CDR3; the V_{L} CDR1 comprising the amino acid sequence as set forth in SEQ ID NO:21; the V_{L} CDR2 comprising the amino acid sequence as set forth in one of SEQ ID NOS:22 or 92-104; the V_{L} CDR3 comprising the amino acid sequence as set forth in SEQ ID NO:23; the V_{H} CDR1 comprising the amino acid sequence as set forth in SEQ ID NO:26; the V_{H} CDR2 comprising the amino acid sequence as set forth in one of SEQ ID NOS:27 or 58-76; the V_{H} CDR3 comprising the amino acid sequence as set forth in one of SEQ ID NOS:28 or 77-91.

In certain embodiments of various methods, pharmaceutical compositions, kits, or uses provided herein, the anti-human TIGIT monoclonal antibody or antigen binding fragment thereof comprises a light chain that comprises a V_{L} CDR1, a V_{L} CDR2, and a V_{L} CDR3, and a heavy chain that comprises a V_{H} CDR1, a V_{H} CDR2, and a V_{H} CDR3; the V_{L} CDR1 comprising the amino acid sequence as set forth in SEQ ID NO:21; the V_{L} CDR2 comprising the amino acid sequence as set forth in SEQ ID NO:104 (X₁X₂KTLAE), wherein X₁ is N, A, V, W, S, T, R, H, G, I, or V, and wherein X₂ is A, N, I, L, T, or V; the V_{L} CDR3 comprising the amino acid sequence as set forth in SEQ ID NO:23; the V_{H} CDR1 comprising the amino acid sequence as set forth in SEQ ID NO:26; the V_{H} CDR2 comprising the amino acid sequence as set forth in SEQ ID NO:76 (YIDPYNX₇X₈AKYX₁₂X₁₃KFX₁₆G), wherein X₇ is D, R, L, K, F, S, Y or V, wherein X₈ is G, R, N, Q, E, L K, S, Y or V, wherein X₁₂ is N, A or S, wherein X₁₃ is E or Q, and wherein X₁₆ is K or Q; and the V_{H} CDR3 comprising the amino acid sequence as set forth in SEQ ID NO:91 (GGPYGX₆YFDV), wherein X₆ is W, A, D, E, F, G, I, K, N, Q, R, S, T, V or Y.

In certain embodiments of various methods, pharmaceutical compositions, kits, or uses provided herein, the V_{H} CDR2 of the anti-human TIGIT monoclonal antibody or antigen binding fragment thereof comprises the amino acid sequence as set forth in SEQ ID NO:58. In some embodiments, the V_{H} CDR2 of the anti-human TIGIT monoclonal antibody or antigen binding fragment thereof comprises the amino acid sequence as set forth in SEQ ID NO:59. In other embodiments, the V_{H} CDR2 of the anti-human TIGIT monoclonal antibody or antigen binding fragment thereof comprises the amino acid sequence as set forth in SEQ ID NO:60. In yet other embodiments, the V_{H} CDR2 of the anti-human TIGIT monoclonal antibody or antigen binding fragment thereof comprises the amino acid sequence as set forth in SEQ ID NO:61. In certain embodiments, the V_{H} CDR2 of the anti-human TIGIT monoclonal antibody or antigen binding fragment thereof comprises the amino acid sequence as set forth in SEQ ID NO:62. In some embodiments, the V_{H} CDR2 of the anti-human TIGIT monoclonal antibody or antigen binding fragment thereof comprises the amino acid sequence as set forth in SEQ ID NO:63. In other embodiments, the V_{H} CDR2 of the anti-human TIGIT monoclonal antibody or antigen binding fragment thereof comprises the amino acid sequence as set forth in SEQ ID NO:64. In yet other embodiments, the V_{H} CDR2 of the anti-human TIGIT monoclonal antibody or antigen binding fragment thereof comprises the amino acid sequence as set forth in SEQ ID NO:65. In certain embodiments, the V_{H} CDR2 of the anti-human TIGIT monoclonal antibody or antigen binding fragment thereof comprises the amino acid sequence as set forth in SEQ ID NO:66. In some embodiments, the V_{H} CDR2 of the anti-human TIGIT monoclonal antibody or antigen binding fragment thereof comprises the amino acid sequence as set forth in SEQ ID NO:67. In other embodiments, the V_{H} CDR2 of the anti-human TIGIT monoclonal antibody or antigen binding fragment thereof comprises the amino acid sequence as set forth in SEQ ID NO:68. In yet other embodiments, the V_{H} CDR2 of the anti-human TIGIT monoclonal antibody or antigen binding fragment thereof comprises the amino acid sequence as set forth in SEQ ID NO:69. In certain embodiments, the V_{H} CDR2 of the anti-human TIGIT monoclonal antibody or antigen binding fragment thereof comprises the amino acid sequence as set forth in SEQ ID NO:70. In some embodiments, the V_{H} CDR2 of the anti-human TIGIT monoclonal antibody or antigen binding fragment thereof comprises the amino acid sequence as set forth in SEQ ID NO:71. In other embodiments, the V_{H} CDR2 of the anti-human TIGIT monoclonal antibody or antigen binding fragment thereof comprises the amino acid sequence as set forth in SEQ ID NO:72. In yet other embodiments, the V_{H} CDR2 of the anti-human TIGIT monoclonal antibody or antigen binding fragment thereof comprises the amino acid sequence as set forth in SEQ ID NO:73. In certain embodiments, the V_{H} CDR2 of the anti-human TIGIT monoclonal antibody or antigen binding fragment thereof comprises the amino acid sequence as set forth in SEQ ID NO:74. In some embodiments, the V_{H} CDR2 of the anti-human TIGIT monoclonal antibody or antigen binding fragment thereof comprises the amino acid sequence as set forth in SEQ ID NO:75.

In certain embodiments of various methods, pharmaceutical compositions, kits, or uses provided herein, the V_{H} CDR3 of the anti-human TIGIT monoclonal antibody or antigen binding fragment thereof comprises the amino acid sequence as set forth in SEQ ID NO:77. In some embodiments, the V_{H} CDR3 of the anti-human TIGIT monoclonal antibody or antigen binding fragment thereof comprises the amino acid sequence as set forth in SEQ ID NO:78. In other embodiments, the V_{H} CDR3 of the anti-human TIGIT monoclonal antibody or antigen binding fragment thereof comprises the amino acid sequence as set forth in SEQ ID NO:79. In yet other embodiments, the V_{H} CDR3 of the anti-human TIGIT monoclonal antibody or antigen binding fragment thereof comprises the amino acid sequence as set forth in SEQ ID NO:80. In certain embodiments, the V_{H} CDR3 of the anti-human TIGIT monoclonal antibody or antigen binding fragment thereof comprises the amino acid sequence as set forth in SEQ ID NO:81. In some embodiments, the V_{H} CDR3 of the anti-human TIGIT monoclonal antibody or antigen binding fragment thereof comprises the amino acid sequence as set forth in SEQ ID NO:82. In other embodiments, the V_{H} CDR3 of the anti-human TIGIT monoclonal antibody or antigen binding fragment thereof comprises the amino acid sequence as set forth in SEQ ID NO:83. In yet other embodiments, the V_{H} CDR3 of the anti-human TIGIT monoclonal antibody or antigen binding fragment thereof comprises the amino acid sequence as set forth in SEQ ID NO:84. In certain embodiments, the V_{H} CDR3 of the anti-human TIGIT monoclonal antibody or antigen binding fragment thereof comprises the amino acid sequence as set forth in SEQ ID NO:85. In some embodiments, the V_{H} CDR3 of the anti-human TIGIT monoclonal antibody or antigen binding fragment thereof comprises the amino acid sequence as set forth in SEQ ID NO:86. In other embodiments, the V_{H} CDR3 of the anti-human TIGIT monoclonal antibody or antigen binding fragment thereof comprises the amino acid sequence as set forth in SEQ ID NO:87. In yet other embodiments, the V_{H} CDR3 of the anti-human TIGIT monoclonal antibody or antigen binding fragment thereof comprises the amino acid sequence as set forth in SEQ ID NO:88. In certain embodiments, the V_{H} CDR3 of the anti-human TIGIT monoclonal antibody or antigen binding fragment thereof comprises the amino acid sequence as set forth in SEQ ID NO:89. In some embodiments, the V_{H} CDR3 of the anti-human TIGIT monoclonal antibody or antigen binding fragment thereof comprises the amino acid sequence as set forth in SEQ ID NO:90.

In certain embodiments of various methods, pharmaceutical compositions, kits, or uses provided herein, the V_{L} CDR2 of the anti-human TIGIT monoclonal antibody or antigen binding fragment thereof comprises the amino acid sequence as set forth in SEQ ID NO:92. In some embodiments, the V_{L} CDR2 of the anti-human TIGIT monoclonal antibody or antigen binding fragment thereof comprises the amino acid sequence as set forth in SEQ ID NO:93. In other embodiments, the V_{L} CDR2 of the anti-human TIGIT monoclonal antibody or antigen binding fragment thereof comprises the amino acid sequence as set forth in SEQ ID NO:94. In yet other embodiments, the V_{L} CDR2 of the anti-human TIGIT monoclonal antibody or antigen binding fragment thereof comprises the amino acid sequence as set forth in SEQ ID NO:95. In certain embodiments, the V_{L} CDR2 of the anti-human TIGIT monoclonal antibody or antigen binding fragment thereof comprises the amino acid sequence as set forth in SEQ ID NO:96. In some embodiments, the V_{L} CDR2 of the anti-human TIGIT monoclonal antibody or antigen binding fragment thereof comprises the amino acid sequence as set forth in SEQ ID NO:97. In other embodiments, the V_{L} CDR2 of the anti-human TIGIT monoclonal antibody or antigen binding fragment thereof comprises the amino acid sequence as set forth in SEQ ID NO:98. In yet other embodiments, the V_{L} CDR2 of the anti-human TIGIT monoclonal antibody or antigen binding fragment thereof comprises the amino acid sequence as set forth in SEQ ID NO:99. In certain embodiments, the V_{L} CDR2 of the anti-human TIGIT monoclonal antibody or antigen binding fragment thereof comprises the amino acid sequence as set forth in SEQ ID NO:100. In some embodiments, the V_{L} CDR2 of the anti-human TIGIT monoclonal antibody or antigen binding fragment thereof comprises the amino acid sequence as set forth in SEQ ID NO:101. In other embodiments, the V_{L} CDR2 of the anti-human TIGIT monoclonal antibody or antigen binding fragment thereof comprises the amino acid sequence as set forth in SEQ ID NO:102. In yet other embodiments, the V_{L} CDR2 of the anti-human TIGIT monoclonal antibody or antigen binding fragment thereof comprises the amino acid sequence as set forth in SEQ ID NO:103.

In various embodiments, the anti-human TIGIT monoclonal antibody or antigen binding fragment thereof comprises a variant of the amino acid sequences disclosed herein. A variant amino acid sequence is identical to the reference sequence except having one, two, three, four, or five amino acid substitutions, deletions, and/or additions. In some embodiments, the substitutions, deletions and/or additions are in the CDRs. In some embodiments, the substitutions, deletions and/or additions are in the framework regions. In certain embodiments, the one, two, three, four, or five of the amino acid substitutions are conservative substitutions.

In one embodiment, the anti-human TIGIT monoclonal antibody or antigen binding fragment thereof has a V_{L} domain with at least 95%, 90%, 85%, 80%, 75% or 50% sequence homology to one of the V_{L} domains described herein, and exhibits specific binding to TIGIT. In another embodiment, the anti-human TIGIT monoclonal antibody or antigen binding fragment thereof has a V_{H} domain with at least 95%, 90%, 85%, 80%, 75% or 50% sequence homology to one of the V_{H} domains described herein, and exhibits specific binding to TIGIT. In yet another embodiment, the anti-human TIGIT monoclonal antibody or antigen binding fragment thereof has a V_{L} domain with at least 95%, 90%, 85%, 80%, 75% or 50% sequence homology to one of the V_{L} domains described herein and a V_{H} domain with at least 95%, 90%, 85%, 80%, 75% or 50% sequence homology to one of the V_{H} domains described herein, and exhibits specific binding to TIGIT.

In one embodiment, the anti-human TIGIT monoclonal antibody or antigen binding fragment thereof has a V_{L} domain having up to 1, 2, 3, 4, 5 or more amino acid substitutions, deletions and/or additions in one of the V_{L} domains described herein, and exhibits specific binding to TIGIT. In another embodiment, the anti-human TIGIT monoclonal antibody or antigen binding fragment thereof has a V_{H} domain having up to 1, 2, 3, 4, 5 or more amino acid substitutions, deletions, and/or additions in one of the V_{H} domains described herein, and exhibits specific binding to TIGIT. In yet another embodiment, the anti-human TIGIT monoclonal antibody or antigen binding fragment thereof has a V_{L} domain having up to 1, 2, 3, 4, 5 or more amino acid substitutions, deletions, and/or additions in one of the V_{L} domains described herein and a V_{H} domain having up to 1, 2, 3, 4, 5 or more amino acid substitutions, deletions, and/or additions in one of the V_{H} domains described herein, and exhibits specific binding to TIGIT.

In various embodiments, the anti-human TIGIT monoclonal antibody or antigen binding fragment thereof is selected from any class of immunoglobulins, including IgM, IgG, IgD, IgA, and IgE. Preferably, the antibody is an IgG antibody. Any isotype of IgG can be used, including IgG1, IgG2, IgG3, and IgG4. Different constant domains may be appended to the V_{L} and V_{H} regions provided herein. For example, if a particular intended use of an antibody (or fragment) of the present invention were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances, an IgG4 constant domain, for example, may be used. In various embodiments, the heavy chain constant domain contains one or more amino acid mutations (e.g., IgG4 with S228P mutation) to generate desired characteristics of the antibody. These desired characteristics include but are not limited to modified effector functions, physical or chemical stability, half-life of antibody, etc.

Ordinarily, amino acid sequence variants of the anti-TIGIT monoclonal antibodies and antigen binding fragments thereof disclosed herein will have an amino acid sequence having at least 75% amino acid sequence identity with the amino acid sequence of a reference antibody or antigen binding fragment (e.g., heavy chain, light chain, V_{H}, V_{L}, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the reference sequence, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3. M. O. Dayhoff(ed.), pp. 353-358, Natl. Biomed. Res. Found., Washington, DC; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

In some embodiments, the anti-human TIGIT monoclonal antibody is a human antibody. In other embodiments, the anti-human TIGIT monoclonal antibody is a humanized antibody.

In some embodiments, the heavy chain of the anti-human TIGIT monoclonal antibody has a human IgG1 backbone. In other embodiments, the heavy chain of the anti-human TIGIT monoclonal antibody has a human IgG2 backbone. In yet other embodiments, the heavy chain of the anti-human TIGIT monoclonal antibody has a human IgG3 backbone. In still other embodiments, the heavy chain of the anti-human TIGIT monoclonal antibody has a human IgG4 backbone.

In some embodiments, the heavy chain of the anti-human TIGIT monoclonal antibody has a human IgG1 variant backbone. In other embodiments, the heavy chain of the anti-human TIGIT monoclonal antibody has a human IgG2 variant backbone. In yet other embodiments, the heavy chain of the anti-human TIGIT monoclonal antibody has a human IgG3 variant backbone. In still other embodiments, the heavy chain of the anti-human TIGIT monoclonal antibody has a human IgG4 variant (e.g., IgG4 with S228P mutation) backbone.

In certain embodiments, the anti-human TIGIT monoclonal antibody is selected from the group consisting of BMS-986207, OMP-313M32, MTIG7192A (RG6058) and PTZ-201 (ASP8374). In one embodiment, the anti-human TIGIT monoclonal antibody is BMS-986207. In another embodiment, the anti-human TIGIT monoclonal antibody is OMP-313M32. In yet another embodiment, the anti-human TIGIT monoclonal antibody is MTIG7192A (RG6058). In still another embodiment, the anti-human TIGIT monoclonal antibody is PTZ-201 (ASP8374).

In some embodiments, the anti-human TIGIT monoclonal antibody can be any antibody, antigen binding fragment thereof, or variant thereof disclosed in WO 2016/028656 and WO 2017/030823, the disclosures of which are incorporated by reference herein in their entireties.

6. Methods of Treating Cancer or Infection Using a Combination of an Anti-PD-1 Monoclonal Antibody, an Anti-LAG3 Monoclonal Antibody, and an Anti-TIGIT Monoclonal Antibody

In another aspect, provided herein are methods of treating cancer (e.g., colorectal cancer) or an infectious disease (e.g., a viral infection) using a combination of an anti-PD-1 monoclonal antibody or antigen binding fragment thereof, an anti-LAG3 monoclonal antibody or antigen binding fragment thereof, and an anti-TIGIT monoclonal antibody or antigen binding fragment thereof,

In certain embodiments, provided herein are methods of treating cancer, comprising administering to a human patient in need thereof:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In some embodiments, the cancer is selected from the group consisting of osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer (e.g., non-small cell lung cancer), gastric cancer, colorectal cancer, cervical cancer, synovial sarcoma, head and neck cancer, squamous cell carcinoma, lymphoma (e.g., DLBCL or NHL), multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, and carcinoid cancer.

In certain embodiments, the cancer is metastatic. In some embodiments, the cancer is relapsed. In other embodiments, the cancer is refractory. In yet other embodiments, the cancer is relapsed and refractory.

In one embodiment, the cancer is osteosarcoma. In another embodiment, the cancer is rhabdomyosarcoma. In yet another embodiment, the cancer is neuroblastoma. In still another embodiment, the cancer is kidney cancer. In one embodiment, the cancer is leukemia. In another embodiment, the cancer is renal transitional cell cancer. In yet another embodiment, the cancer is bladder cancer. In still another embodiment, the cancer is Wilm's cancer. In one embodiment, the cancer is ovarian cancer. In another embodiment, the cancer is pancreatic cancer. In yet another embodiment, the cancer is breast cancer. In still another embodiment, the cancer is prostate cancer. In one embodiment, the cancer is bone cancer. In another embodiment, the cancer is lung cancer. In yet another embodiment, the cancer is non-small cell lung cancer. In still another embodiment, the cancer is gastric cancer. In one embodiment, the cancer is colorectal cancer. In another embodiment, the cancer is cervical cancer. In yet another embodiment, the cancer is synovial sarcoma. In still another embodiment, the cancer is head and neck cancer. In one embodiment, the cancer is squamous cell carcinoma. In another embodiment, the cancer is lymphoma. In one embodiment, the cancer is DLBCL. In another embodiment, the cancer is NHL. In yet another embodiment, the cancer is multiple myeloma. In still another embodiment, the cancer is renal cell cancer. In one embodiment, the cancer is retinoblastoma. In another embodiment, the cancer is hepatoblastoma. In yet another embodiment, the cancer is hepatocellular carcinoma. In still another embodiment, the cancer is melanoma. In one embodiment, the cancer is rhabdoid tumor of the kidney. In another embodiment, the cancer is Ewing's sarcoma. In yet another embodiment, the cancer is chondrosarcoma. In still another embodiment, the cancer is brain cancer. In one embodiment, the cancer is glioblastoma. In another embodiment, the cancer is meningioma. In yet another embodiment, the cancer is pituitary adenoma. In still another embodiment, the cancer is vestibular schwannoma. In one embodiment, the cancer is primitive neuroectodermal tumor. In another embodiment, the cancer is medulloblastoma. In yet another embodiment, the cancer is astrocytoma. In still another embodiment, the cancer is anaplastic astrocytoma. In one embodiment, the cancer is oligodendroglioma. In another embodiment, the cancer is ependymoma. In yet another embodiment, the cancer is choroid plexus papilloma. In still another embodiment, the cancer is polycythemia vera. In one embodiment, the cancer is thrombocythemia. In another embodiment, the cancer is idiopathic myelfibrosis. In yet another embodiment, the cancer is soft tissue sarcoma. In still another embodiment, the cancer is thyroid cancer. In one embodiment, the cancer is endometrial cancer. In another embodiment, the cancer is carcinoid cancer. In yet another embodiment, the cancer is refractory head and neck cancer. In still another embodiment, the cancer is relapsed/refractory NHL (rrNHL). In another embodiment, the cancer is naïve rrNHL. In yet still another embodiment, the cancer is PD-1 refractory rrNHL.

In certain embodiments, provided herein is a method of treating colorectal cancer, comprising administering to a human patient in need thereof:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In some embodiments, provided herein is a method of treating gastric cancer, comprising administering to a human patient in need thereof:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In other embodiments, provided herein is a method of treating head and neck cancer, comprising administering to a human patient in need thereof:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In yet other embodiments, provided herein is a method of treating refractory head and neck cancer, comprising administering to a human patient in need thereof:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In still other embodiments, provided herein is a method of treating lung cancer, comprising administering to a human patient in need thereof:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In certain embodiments, provided herein is a method of treating non-small cell lung cancer, comprising administering to a human patient in need thereof:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In some embodiments, provided herein is a method of treating breast cancer, comprising administering to a human patient in need thereof:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In other embodiments, provided herein is a method of treating cervical cancer, comprising administering to a human patient in need thereof:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In yet other embodiments, provided herein is a method of treating ovarian cancer, comprising administering to a human patient in need thereof:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In still other embodiments, provided herein is a method of treating DLBCL, comprising administering to a human patient in need thereof:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In certain embodiments, provided herein is a method of treating NHL, comprising administering to a human patient in need thereof:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In some embodiments, provided herein is a method of treating rrNHL, comprising administering to a human patient in need thereof:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In other embodiments, provided herein is a method of treating naïve rrNHL, comprising administering to a human patient in need thereof:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In yet other embodiments, provided herein is a method of treating PD-1 refractory rrNHL, comprising administering to a human patient in need thereof:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In certain embodiments, the method of treating cancer comprises administering to a human patient in need thereof:
(a) an anti-human PD-1 monoclonal antibody or antigen binding fragment thereof as disclosed in Section V.3;
(b) an anti-human LAG3 monoclonal antibody or antigen binding fragment thereof as disclosed in Section V.4; and
(c) an anti-human TIGIT monoclonal antibody or antigen binding fragment thereof as disclosed in Section V.5.

In one specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In another specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In yet another specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In one specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In another specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In yet another specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In one specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In another specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In yet another specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In one specific embodiment, the method of treating cancer comprises administering to a human patient in need thereof: (a) an anti-human PD-1 monoclonal antibody or antigen binding fragment thereof that comprises a V_{L} region comprising an amino acid sequence as set forth in SEQ ID NO:4, and a V_{H} region comprising an amino acid sequence as set forth in SEQ ID NO:9; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In another specific embodiment, the method of treating cancer comprises administering to a human patient in need thereof: (a) an anti-human PD-1 monoclonal antibody or antigen binding fragment thereof that comprises a V_{L} region comprising an amino acid sequence as set forth in SEQ ID NO:4, and a V_{H} region comprising an amino acid sequence as set forth in SEQ ID NO:9; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In yet another specific embodiment, the method of treating cancer comprises administering to a human patient in need thereof: (a) an anti-human PD-1 monoclonal antibody or antigen binding fragment thereof that comprises a V_{L} region comprising an amino acid sequence as set forth in SEQ ID NO:4, and a V_{H} region comprising an amino acid sequence as set forth in SEQ ID NO:9; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In one specific embodiment, the method of treating cancer comprises administering to a human patient in need thereof: (a) an anti-human PD-1 monoclonal antibody or antigen binding fragment thereof that comprises a V_{L} region comprising an amino acid sequence as set forth in SEQ ID NO:4, and a V_{H} region comprising an amino acid sequence as set forth in SEQ ID NO:9; (b) an anti-human LAG3 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In another specific embodiment, the method of treating cancer comprises administering to a human patient in need thereof: (a) an anti-human PD-1 monoclonal antibody or antigen binding fragment thereof that comprises a V_{L} region comprising an amino acid sequence as set forth in SEQ ID NO:4, and a V_{H} region comprising an amino acid sequence as set forth in SEQ ID NO:9; (b) an anti-human LAG3 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In yet another specific embodiment, the method of treating cancer comprises administering to a human patient in need thereof: (a) an anti-human PD-1 monoclonal antibody or antigen binding fragment thereof that comprises a V_{L} region comprising an amino acid sequence as set forth in SEQ ID NO:4, and a V_{H} region comprising an amino acid sequence as set forth in SEQ ID NO:9; (b) an anti-human LAG3 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In one specific embodiment, the method of treating cancer comprises administering to a human patient in need thereof: (a) an anti-human PD-1 monoclonal antibody or antigen binding fragment thereof that comprises a V_{L} region comprising an amino acid sequence as set forth in SEQ ID NO:4, and a V_{H} region comprising an amino acid sequence as set forth in SEQ ID NO:9; (b) an anti-human LAG3 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In another specific embodiment, the method of treating cancer comprises administering to a human patient in need thereof: (a) an anti-human PD-1 monoclonal antibody or antigen binding fragment thereof that comprises a V_{L} region comprising an amino acid sequence as set forth in SEQ ID NO:4, and a V_{H} region comprising an amino acid sequence as set forth in SEQ ID NO:9; (b) an anti-human LAG3 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In yet another specific embodiment, the method of treating cancer comprises administering to a human patient in need thereof: (a) an anti-human PD-1 monoclonal antibody or antigen binding fragment thereof that comprises a V_{L} region comprising an amino acid sequence as set forth in SEQ ID NO:4, and a V_{H} region comprising an amino acid sequence as set forth in SEQ ID NO:9; (b) an anti-human LAG3 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In one specific embodiment, the method of treating cancer comprises administering to a human patient in need thereof: (a) an anti-human PD-1 monoclonal antibody or antigen binding fragment thereof that comprises a light chain comprising or consisting of an amino acid sequence as set forth in SEQ ID NO:5 and a heavy chain comprising or consisting of an amino acid sequence as set forth in SEQ ID NO:10; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In another specific embodiment, the method of treating cancer comprises administering to a human patient in need thereof: (a) an anti-human PD-1 monoclonal antibody or antigen binding fragment thereof that comprises a light chain comprising or consisting of an amino acid sequence as set forth in SEQ ID NO:5 and a heavy chain comprising or consisting of an amino acid sequence as set forth in SEQ ID NO:10; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In yet another specific embodiment, the method of treating cancer comprises administering to a human patient in need thereof: (a) an anti-human PD-1 monoclonal antibody or antigen binding fragment thereof that comprises a light chain comprising or consisting of an amino acid sequence as set forth in SEQ ID NO:5 and a heavy chain comprising or consisting of an amino acid sequence as set forth in SEQ ID NO:10; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In one specific embodiment, the method of treating cancer comprises administering to a human patient in need thereof: (a) an anti-human PD-1 monoclonal antibody or antigen binding fragment thereof that comprises a light chain comprising or consisting of an amino acid sequence as set forth in SEQ ID NO:5 and a heavy chain comprising or consisting of an amino acid sequence as set forth in SEQ ID NO:10; (b) an anti-human LAG3 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In another specific embodiment, the method of treating cancer comprises administering to a human patient in need thereof: (a) an anti-human PD-1 monoclonal antibody or antigen binding fragment thereof that comprises a light chain comprising or consisting of an amino acid sequence as set forth in SEQ ID NO:5 and a heavy chain comprising or consisting of an amino acid sequence as set forth in SEQ ID NO:10; (b) an anti-human LAG3 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In yet another specific embodiment, the method of treating cancer comprises administering to a human patient in need thereof: (a) an anti-human PD-1 monoclonal antibody or antigen binding fragment thereof that comprises a light chain comprising or consisting of an amino acid sequence as set forth in SEQ ID NO:5 and a heavy chain comprising or consisting of an amino acid sequence as set forth in SEQ ID NO:10; (b) an anti-human LAG3 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In one specific embodiment, the method of treating cancer comprises administering to a human patient in need thereof: (a) an anti-human PD-1 monoclonal antibody or antigen binding fragment thereof that comprises a light chain comprising or consisting of an amino acid sequence as set forth in SEQ ID NO:5 and a heavy chain comprising or consisting of an amino acid sequence as set forth in SEQ ID NO:10; (b) an anti-human LAG3 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively. In another specific embodiment, the method of treating cancer comprises administering to a human patient in need thereof: (a) an anti-human PD-1 monoclonal antibody or antigen binding fragment thereof that comprises a light chain comprising or consisting of an amino acid sequence as set forth in SEQ ID NO:5 and a heavy chain comprising or consisting of an amino acid sequence as set forth in SEQ ID NO:10; (b) an anti-human LAG3 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In yet another specific embodiment, the method of treating cancer comprises administering to a human patient in need thereof: (a) an anti-human PD-1 monoclonal antibody or antigen binding fragment thereof that comprises a light chain comprising or consisting of an amino acid sequence as set forth in SEQ ID NO:5 and a heavy chain comprising or consisting of an amino acid sequence as set forth in SEQ ID NO:10; (b) an anti-human LAG3 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In one specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In another specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In yet another specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In one specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In another specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In yet another specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In one specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In another specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In yet another specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In one specific embodiment, the method of treating cancer comprises administering to a human patient in need thereof: (a) an anti-human PD-1 monoclonal antibody or antigen binding fragment thereof that comprises a V_{L} region comprising an amino acid sequence as set forth in SEQ ID NO:34, and a V_{H} region comprising an amino acid sequence as set forth in SEQ ID NO:39; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In another specific embodiment, the method of treating cancer comprises administering to a human patient in need thereof: (a) an anti-human PD-1 monoclonal antibody or antigen binding fragment thereof that comprises a V_{L} region comprising an amino acid sequence as set forth in SEQ ID NO:34, and a V_{H} region comprising an amino acid sequence as set forth in SEQ ID NO:39; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In yet another specific embodiment, the method of treating cancer comprises administering to a human patient in need thereof: (a) an anti-human PD-1 monoclonal antibody or antigen binding fragment thereof that comprises a V_{L} region comprising an amino acid sequence as set forth in SEQ ID NO:34, and a V_{H} region comprising an amino acid sequence as set forth in SEQ ID NO:39; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In one specific embodiment, the method of treating cancer comprises administering to a human patient in need thereof: (a) an anti-human PD-1 monoclonal antibody or antigen binding fragment thereof that comprises a V_{L} region comprising an amino acid sequence as set forth in SEQ ID NO:34, and a V_{H} region comprising an amino acid sequence as set forth in SEQ ID NO:39; (b) an anti-human LAG3 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In another specific embodiment, the method of treating cancer comprises administering to a human patient in need thereof: (a) an anti-human PD-1 monoclonal antibody or antigen binding fragment thereof that comprises a V_{L} region comprising an amino acid sequence as set forth in SEQ ID NO:34, and a V_{H} region comprising an amino acid sequence as set forth in SEQ ID NO:39; (b) an anti-human LAG3 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In yet another specific embodiment, the method of treating cancer comprises administering to a human patient in need thereof: (a) an anti-human PD-1 monoclonal antibody or antigen binding fragment thereof that comprises a V_{L} region comprising an amino acid sequence as set forth in SEQ ID NO:34, and a V_{H} region comprising an amino acid sequence as set forth in SEQ ID NO:39; (b) an anti-human LAG3 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In one specific embodiment, the method of treating cancer comprises administering to a human patient in need thereof: (a) an anti-human PD-1 monoclonal antibody or antigen binding fragment thereof that comprises a V_{L} region comprising an amino acid sequence as set forth in SEQ ID NO:34, and a V_{H} region comprising an amino acid sequence as set forth in SEQ ID NO:39; (b) an anti-human LAG3 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In another specific embodiment, the method of treating cancer comprises administering to a human patient in need thereof: (a) an anti-human PD-1 monoclonal antibody or antigen binding fragment thereof that comprises a V_{L} region comprising an amino acid sequence as set forth in SEQ ID NO:34, and a V_{H} region comprising an amino acid sequence as set forth in SEQ ID NO:39; (b) an anti-human LAG3 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In yet another specific embodiment, the method of treating cancer comprises administering to a human patient in need thereof: (a) an anti-human PD-1 monoclonal antibody or antigen binding fragment thereof that comprises a V_{L} region comprising an amino acid sequence as set forth in SEQ ID NO:34, and a V_{H} region comprising an amino acid sequence as set forth in SEQ ID NO:39; (b) an anti-human LAG3 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In one specific embodiment, the method of treating cancer comprises administering to a human patient in need thereof: (a) an anti-human PD-1 monoclonal antibody or antigen binding fragment thereof that comprises a light chain comprising or consisting of an amino acid sequence as set forth in SEQ ID NO:35 and a heavy chain comprising or consisting of an amino acid sequence as set forth in SEQ ID NO:40; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In another specific embodiment, the method of treating cancer comprises administering to a human patient in need thereof: (a) an anti-human PD-1 monoclonal antibody or antigen binding fragment thereof that comprises a light chain comprising or consisting of an amino acid sequence as set forth in SEQ ID NO:35 and a heavy chain comprising or consisting of an amino acid sequence as set forth in SEQ ID NO:40; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In yet another specific embodiment, the method of treating cancer comprises administering to a human patient in need thereof: (a) an anti-human PD-1 monoclonal antibody or antigen binding fragment thereof that comprises a light chain comprising or consisting of an amino acid sequence as set forth in SEQ ID NO:35 and a heavy chain comprising or consisting of an amino acid sequence as set forth in SEQ ID NO:40; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In one specific embodiment, the method of treating cancer comprises administering to a human patient in need thereof: (a) an anti-human PD-1 monoclonal antibody or antigen binding fragment thereof that comprises a light chain comprising or consisting of an amino acid sequence as set forth in SEQ ID NO:35 and a heavy chain comprising or consisting of an amino acid sequence as set forth in SEQ ID NO:40; (b) an anti-human LAG3 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In another specific embodiment, the method of treating cancer comprises administering to a human patient in need thereof: (a) an anti-human PD-1 monoclonal antibody or antigen binding fragment thereof that comprises a light chain comprising or consisting of an amino acid sequence as set forth in SEQ ID NO:35 and a heavy chain comprising or consisting of an amino acid sequence as set forth in SEQ ID NO:40; (b) an anti-human LAG3 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In yet another specific embodiment, the method of treating cancer comprises administering to a human patient in need thereof: (a) an anti-human PD-1 monoclonal antibody or antigen binding fragment thereof that comprises a light chain comprising or consisting of an amino acid sequence as set forth in SEQ ID NO:35 and a heavy chain comprising or consisting of an amino acid sequence as set forth in SEQ ID NO:40; (b) an anti-human LAG3 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In one specific embodiment, the method of treating cancer comprises administering to a human patient in need thereof: (a) an anti-human PD-1 monoclonal antibody or antigen binding fragment thereof that comprises a light chain comprising or consisting of an amino acid sequence as set forth in SEQ ID NO:35 and a heavy chain comprising or consisting of an amino acid sequence as set forth in SEQ ID NO:40; (b) an anti-human LAG3 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In another specific embodiment, the method of treating cancer comprises administering to a human patient in need thereof: (a) an anti-human PD-1 monoclonal antibody or antigen binding fragment thereof that comprises a light chain comprising or consisting of an amino acid sequence as set forth in SEQ ID NO:35 and a heavy chain comprising or consisting of an amino acid sequence as set forth in SEQ ID NO:40; (b) an anti-human LAG3 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In yet another specific embodiment, the method of treating cancer comprises administering to a human patient in need thereof: (a) an anti-human PD-1 monoclonal antibody or antigen binding fragment thereof that comprises a light chain comprising or consisting of an amino acid sequence as set forth in SEQ ID NO:35 and a heavy chain comprising or consisting of an amino acid sequence as set forth in SEQ ID NO:40; (b) an anti-human LAG3 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In one specific embodiment, the method of treating cancer comprises administering to a human patient in need thereof: (a) cemiplimab; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) pidilizumab; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) AMP-514; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) PDR001; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) BGB-A317; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) MGA012; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) BMS-986016; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) REGN3767; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) an anti-human PD-1 monoclonal antibody or antigen binding fragment thereof comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) LAG525; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) GSK2813781; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) BMS-986016; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) REGN3767; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) LAG525; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) GSK2813781; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) BMS-986207.

In one specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) OMP-313M32.

In one specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) MTIG7192A (RG6058).

In one specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) PTZ-201 (ASP8374).

In one specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) BMS-986207.

In one specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) OMP-313M32.

In one specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) MTIG7192A (RG6058).

In one specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) PTZ-201 (ASP8374).

In one specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) BMS-986016; and (c) BMS-986207.

In another specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) BMS-986016; and (c) OMP-313M32.

In yet another specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) BMS-986016; and (c) MTIG7192A (RG6058).

In still another embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) BMS-986016; and (c) PTZ-201 (ASP8374).

In one specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) REGN3767; and (c) BMS-986207.

In another specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) REGN3767; and (c) OMP-313M32.

In yet another specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) REGN3767; and (c) MTIG7192A (RG6058).

In still another embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) REGN3767; and (c) PTZ-201 (ASP8374).

In one specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) LAG525; and (c) BMS-986207.

In another specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} III CDR1, a V_{H} CDR2, and a V_{H} CDR3 comprising amino acid sequences as set forth in SEQ ID NOS:6, 7, and 8, respectively; (b) LAG525; and (c) OMP-313M32.

In yet another specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) LAG525; and (c) MTIG7192A (RG6058).

In still another embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) LAG525; and (c) PTZ-201 (ASP8374).

In one specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) GSK2813781; and (c) BMS-986207.

In another specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) GSK2813781; and (c) OMP-313M32.

In yet another specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) GSK2813781; and (c) MTIG7192A (RG6058).

In still another embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1,2, and 3, respectively, and a V_{H} CDR1, a V_{H} CDR2, and a V_{H} CDR3 comprising amino acid sequences as set forth in SEQ ID NOS:6, 7, and 8, respectively; (b) GSK2813781; and (c) PTZ-201 (ASP8374).

In one specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) BMS-986016; and (c) BMS-986207.

In another specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) BMS-986016; and (c) OMP-313M32.

In yet another specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) BMS-986016; and (c) MTIG7192A (RG6058).

In still another embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) BMS-986016; and (c) PTZ-201 (ASP8374).

In one specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) REGN3767; and (c) BMS-986207.

In another specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) REGN3767; and (c) OMP-313M32.

In yet another specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) REGN3767; and (c) MTIG7192A (RG6058).

In still another embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) REGN3767; and (c) PTZ-201 (ASP8374).

In one specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) LAG525; and (c) BMS-986207.

In another specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) LAG525; and (c) OMP-313M32.

In yet another specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) LAG525; and (c) MTIG7192A (RG6058).

In still another embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) LAG525; and (c) PTZ-201 (ASP8374).

In one specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) GSK2813781; and (c) BMS-986207.

In another specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) GSK2813781; and (c) OMP-313M32.

In yet another specific embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) GSK2813781; and (c) MTIG7192A (RG6058).

In still another embodiment, the method of treating cancer comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) GSK2813781; and (c) PTZ-201 (ASP8374).

In one specific embodiment, the method of treating cancer comprises administering to a human patient in need thereof: (a) cemiplimab; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) BMS-986207.

In another specific embodiment, the method of treating cancer comprises administering to a human patient in need thereof: (a) cemiplimab; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) OMP-313M32.

In yet another specific embodiment, the method of treating cancer comprises administering to a human patient in need thereof: (a) cemiplimab; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) MTIG7192A (RG6058).

In still another embodiment, the method of treating cancer comprises administering to a human patient in need thereof: (a) cemiplimab; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) PTZ-201 (ASP8374).

In one specific embodiment, the method of treating cancer comprises administering to a human patient in need thereof: (a) pidilizumab; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) BMS-986207.

In another specific embodiment, the method of treating cancer comprises administering to a human patient in need thereof: (a) pidilizumab; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) OMP-313M32.

In yet another specific embodiment, the method of treating cancer comprises administering to a human patient in need thereof: (a) pidilizumab; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) MTIG7192A (RG6058).

In still another embodiment, the method of treating cancer comprises administering to a human patient in need thereof: (a) pidilizumab; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) PTZ-201 (ASP8374).

In one specific embodiment, the method of treating cancer comprises administering to a human patient in need thereof: (a) AMP-514; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) BMS-986207.

In another specific embodiment, the method of treating cancer comprises administering to a human patient in need thereof: (a) AMP-514; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) OMP-313M32. In yet another specific embodiment, the method of treating cancer comprises administering to a human patient in need thereof: (a) AMP-514; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) MTIG7192A (RG6058).

In still another embodiment, the method of treating cancer comprises administering to a human patient in need thereof: (a) AMP-514; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) PTZ-201 (ASP8374).

In one specific embodiment, the method of treating cancer comprises administering to a human patient in need thereof: (a) PDR001; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) BMS-986207.

In another specific embodiment, the method of treating cancer comprises administering to a human patient in need thereof: (a) PDR001; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) OMP-313M32.

In yet another specific embodiment, the method of treating cancer comprises administering to a human patient in need thereof: (a) PDR001; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) MTIG7192A (RG6058).

In still another embodiment, the method of treating cancer comprises administering to a human patient in need thereof: (a) PDR001; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) PTZ-201 (ASP8374).

In one specific embodiment, the method of treating cancer comprises administering to a human patient in need thereof: (a) BGB-A317; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) BMS-986207.

In another specific embodiment, the method of treating cancer comprises administering to a human patient in need thereof: (a) BGB-A317; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) OMP-313M32.

In yet another specific embodiment, the method of treating cancer comprises administering to a human patient in need thereof: (a) BGB-A317; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) MTIG7192A (RG6058).

In still another embodiment, the method of treating cancer comprises administering to a human patient in need thereof: (a) BGB-A317; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) PTZ-201 (ASP8374).

In one specific embodiment, the method of treating cancer comprises administering to a human patient in need thereof: (a) MGA012; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) BMS-986207.

In another specific embodiment, the method of treating cancer comprises administering to a human patient in need thereof: (a) MGA012; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) OMP-313M32.

In yet another specific embodiment, the method of treating cancer comprises administering to a human patient in need thereof: (a) MGA012; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) MTIG7192A (RG6058).

In still another embodiment, the method of treating cancer comprises administering to a human patient in need thereof: (a) MGA012; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) PTZ-201 (ASP8374).

In one specific embodiment, the method of treating cancer comprises administering to a human patient in need thereof: (a) cemiplimab; (b) BMS-986016; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) cemiplimab; (b) REGN3767; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) cemiplimab; (b) LAG525; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) cemiplimab; (b) GSK2813781; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) pidilizumab; (b) BMS-986016; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) pidilizumab; (b) REGN3767; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) pidilizumab; (b) LAG525; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively. In one specific embodiment, the method of treating cancer comprises administering to a human patient in need thereof: (a) pidilizumab; (b) GSK2813781; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) AMP-514; (b) BMS-986016; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) AMP-514; (b) REGN3767; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) AMP-514; (b) LAG525; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) AMP-514; (b) GSK2813781; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) PDR001; (b) BMS-986016; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) PDR001; (b) REGN3767; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) PDR001; (b) LAG525; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) PDR001; (b) GSK2813781; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) BGB-A317; (b) BMS-986016; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) BGB-A317; (b) REGN3767; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) BGB-A317; (b) LAG525; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) BGB-A317; (b) GSK2813781 and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) MGA012; (b) BMS-986016; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) MGA012; (b) REGN3767; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) MGA012; (b) LAG525; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: (a) MGA012; (b) GSK2813781; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of treating cancer comprises administering to a human patient in need thereof: pembrolizumab, BMS-986016, and BMS-986207.

In another specific embodiment, the method of treating cancer comprises administering to a human patient in need thereof: pembrolizumab, BMS-986016, and OMP-313M32.

In yet another specific embodiment, the method of treating cancer comprises administering to a human patient in need thereof: pembrolizumab, BMS-986016, and MTIG7192A (RG6058).

In still another embodiment, the method of treating cancer comprises administering to a human patient in need thereof: pembrolizumab, BMS-986016, and PTZ-201 (ASP8374).

In one specific embodiment, the method of treating cancer comprises administering to a human patient in need thereof: pembrolizumab, REGN3767, and BMS-986207.

In another specific embodiment, the method of treating cancer comprises administering to a human patient in need thereof: pembrolizumab, REGN3767, and OMP-313M32.

In yet another specific embodiment, the method of treating cancer comprises administering to a human patient in need thereof: pembrolizumab, REGN3767, and MTIG7192A (RG6058).

In still another embodiment, the method of treating cancer comprises administering to a human patient in need thereof: pembrolizumab, REGN3767, and PTZ-201 (ASP8374).

In one specific embodiment, the method of treating cancer comprises administering to a human patient in need thereof: pembrolizumab, LAG525, and BMS-986207.

In another specific embodiment, the method of treating cancer comprises administering to a human patient in need thereof: pembrolizumab, LAG525, and OMP-313M32.

In yet another specific embodiment, the method of treating cancer comprises administering to a human patient in need thereof: pembrolizumab, LAG525, and MTIG7192A (RG6058).

In still another embodiment, the method of treating cancer comprises administering to a human patient in need thereof: pembrolizumab, LAG525, and PTZ-201 (ASP8374).

In one specific embodiment, the method of treating cancer comprises administering to a human patient in need thereof: pembrolizumab, GSK2813781, and BMS-986207.

In another specific embodiment, the method of treating cancer comprises administering to a human patient in need thereof: pembrolizumab, GSK2813781, and OMP-313M32.

In yet another specific embodiment, the method of treating cancer comprises administering to a human patient in need thereof: pembrolizumab, GSK2813781, and MTIG7192A (RG6058).

In still another embodiment, the method of treating cancer comprises administering to a human patient in need thereof: pembrolizumab, GSK2813781, and PTZ-201 (ASP8374).

In one specific embodiment, the method of treating cancer comprises administering to a human patient in need thereof: nivolumab, BMS-986016, and BMS-986207.

In another specific embodiment, the method of treating cancer comprises administering to a human patient in need thereof: nivolumab, BMS-986016, and OMP-313M32.

In yet another specific embodiment, the method of treating cancer comprises administering to a human patient in need thereof: nivolumab, BMS-986016, and MTIG7192A (RG6058).

In still another embodiment, the method of treating cancer comprises administering to a human patient in need thereof: nivolumab, BMS-986016, and PTZ-201 (ASP8374).

In one specific embodiment, the method of treating cancer comprises administering to a human patient in need thereof: nivolumab, REGN3767, and BMS-986207.

In another specific embodiment, the method of treating cancer comprises administering to a human patient in need thereof: nivolumab, REGN3767, and OMP-313M32.

In yet another specific embodiment, the method of treating cancer comprises administering to a human patient in need thereof: nivolumab, REGN3767, and MTIG7192A (RG6058).

In still another embodiment, the method of treating cancer comprises administering to a human patient in need thereof: nivolumab, REGN3767, and PTZ-201 (ASP8374).

In one specific embodiment, the method of treating cancer comprises administering to a human patient in need thereof: nivolumab, LAG525, and BMS-986207.

In another specific embodiment, the method of treating cancer comprises administering to a human patient in need thereof: nivolumab, LAG525, and OMP-313M32.

In yet another specific embodiment, the method of treating cancer comprises administering to a human patient in need thereof: nivolumab, LAG525, and MTIG7192A (RG6058).

In still another embodiment, the method of treating cancer comprises administering to a human patient in need thereof: nivolumab, LAG525, and PTZ-201 (ASP8374).

In one specific embodiment, the method of treating cancer comprises administering to a human patient in need thereof: nivolumab, GSK2813781, and BMS-986207.

In another specific embodiment, the method of treating cancer comprises administering to a human patient in need thereof: nivolumab, GSK2813781, and OMP-313M32.

In yet another specific embodiment, the method of treating cancer comprises administering to a human patient in need thereof: nivolumab, GSK2813781, and MTIG7192A (RG6058).

In still another embodiment, the method of treating cancer comprises administering to a human patient in need thereof: nivolumab, GSK2813781, and PTZ-201 (ASP8374).

In one specific embodiment, the method of treating cancer comprises administering to a human patient in need thereof: cemiplimab, BMS-986016, and BMS-986207.

In another specific embodiment, the method of treating cancer comprises administering to a human patient in need thereof: cemiplimab, BMS-986016, and OMP-313M32.

In yet another specific embodiment, the method of treating cancer comprises administering to a human patient in need thereof: cemiplimab, BMS-986016, and MTIG7192A (RG6058).

In still another embodiment, the method of treating cancer comprises administering to a human patient in need thereof: cemiplimab, BMS-986016, and PTZ-201 (ASP8374).

In one specific embodiment, the method of treating cancer comprises administering to a human patient in need thereof: cemiplimab, REGN3767, and BMS-986207.

In another specific embodiment, the method of treating cancer comprises administering to a human patient in need thereof: cemiplimab, REGN3767, and OMP-313M32.

In yet another specific embodiment, the method of treating cancer comprises administering to a human patient in need thereof: cemiplimab, REGN3767, and MTIG7192A (RG6058).

In still another embodiment, the method of treating cancer comprises administering to a human patient in need thereof: cemiplimab, REGN3767, and PTZ-201 (ASP8374).

In one specific embodiment, the method of treating cancer comprises administering to a human patient in need thereof: cemiplimab, LAG525, and BMS-986207.

In another specific embodiment, the method of treating cancer comprises administering to a human patient in need thereof: cemiplimab, LAG525, and OMP-313M32.

In yet another specific embodiment, the method of treating cancer comprises administering to a human patient in need thereof: cemiplimab, LAG525, and MTIG7192A (RG6058).

In still another embodiment, the method of treating cancer comprises administering to a human patient in need thereof: cemiplimab, LAG525, and PTZ-201 (ASP8374).

In one specific embodiment, the method of treating cancer comprises administering to a human patient in need thereof: cemiplimab, GSK2813781, and BMS-986207.

In another specific embodiment, the method of treating cancer comprises administering to a human patient in need thereof: cemiplimab, GSK2813781, and OMP-313M32.

In yet another specific embodiment, the method of treating cancer comprises administering to a human patient in need thereof: cemiplimab, GSK2813781, and MTIG7192A (RG6058).

In still another embodiment, the method of treating cancer comprises administering to a human patient in need thereof: cemiplimab, GSK2813781, and PTZ-201 (ASP8374).

In one specific embodiment, the method of treating cancer comprises administering to a human patient in need thereof: pidilizumab, BMS-986016, and BMS-986207.

In another specific embodiment, the method of treating cancer comprises administering to a human patient in need thereof: pidilizumab, BMS-986016, and OMP-313M32.

In yet another specific embodiment, the method of treating cancer comprises administering to a human patient in need thereof: pidilizumab, BMS-986016, and MTIG7192A (RG6058).

In still another embodiment, the method of treating cancer comprises administering to a human patient in need thereof: pidilizumab, BMS-986016, and PTZ-201 (ASP8374).

In one specific embodiment, the method of treating cancer comprises administering to a human patient in need thereof: pidilizumab, REGN3767, and BMS-986207.

In another specific embodiment, the method of treating cancer comprises administering to a human patient in need thereof: pidilizumab, REGN3767, and OMP-313M32.

In yet another specific embodiment, the method of treating cancer comprises administering to a human patient in need thereof: pidilizumab, REGN3767, and MTIG7192A (RG6058).

In still another embodiment, the method of treating cancer comprises administering to a human patient in need thereof: pidilizumab, REGN3767, and PTZ-201 (ASP8374).

In one specific embodiment, the method of treating cancer comprises administering to a human patient in need thereof: pidilizumab, LAG525, and BMS-986207.

In another specific embodiment, the method of treating cancer comprises administering to a human patient in need thereof: pidilizumab, LAG525, and OMP-313M32.

In yet another specific embodiment, the method of treating cancer comprises administering to a human patient in need thereof: pidilizumab, LAG525, and MTIG7192A (RG6058).

In still another embodiment, the method of treating cancer comprises administering to a human patient in need thereof: pidilizumab, LAG525, and PTZ-201 (ASP8374).

In one specific embodiment, the method of treating cancer comprises administering to a human patient in need thereof: pidilizumab, GSK2813781, and BMS-986207.

In another specific embodiment, the method of treating cancer comprises administering to a human patient in need thereof: pidilizumab, GSK2813781, and OMP-313M32.

In yet another specific embodiment, the method of treating cancer comprises administering to a human patient in need thereof: pidilizumab, GSK2813781, and MTIG7192A (RG6058).

In still another embodiment, the method of treating cancer comprises administering to a human patient in need thereof: pidilizumab, GSK2813781, and PTZ-201 (ASP8374).

In one specific embodiment, the method of treating cancer comprises administering to a human patient in need thereof: AMP-514, BMS-986016, and BMS-986207.

In another specific embodiment, the method of treating cancer comprises administering to a human patient in need thereof: AMP-514, BMS-986016, and OMP-313M32.

In yet another specific embodiment, the method of treating cancer comprises administering to a human patient in need thereof: AMP-514, BMS-986016, and MTIG7192A (RG6058).

In still another embodiment, the method of treating cancer comprises administering to a human patient in need thereof: AMP-514, BMS-986016, and PTZ-201 (ASP8374).

In one specific embodiment, the method of treating cancer comprises administering to a human patient in need thereof: AMP-514, REGN3767, and BMS-986207.

In another specific embodiment, the method of treating cancer comprises administering to a human patient in need thereof: AMP-514, REGN3767, and OMP-313M32.

In yet another specific embodiment, the method of treating cancer comprises administering to a human patient in need thereof: AMP-514, REGN3767, and MTIG7192A (RG6058).

In still another embodiment, the method of treating cancer comprises administering to a human patient in need thereof: AMP-514, REGN3767, and PTZ-201 (ASP8374).

In one specific embodiment, the method of treating cancer comprises administering to a human patient in need thereof: AMP-514, LAG525, and BMS-986207.

In another specific embodiment, the method of treating cancer comprises administering to a human patient in need thereof: AMP-514, LAG525, and OMP-313M32.

In yet another specific embodiment, the method of treating cancer comprises administering to a human patient in need thereof: AMP-514, LAG525, and MTIG7192A (RG6058).

In still another embodiment, the method of treating cancer comprises administering to a human patient in need thereof: AMP-514, LAG525, and PTZ-201 (ASP8374).

In one specific embodiment, the method of treating cancer comprises administering to a human patient in need thereof: AMP-514, GSK2813781, and BMS-986207.

In another specific embodiment, the method of treating cancer comprises administering to a human patient in need thereof: AMP-514, GSK2813781, and OMP-313M32.

In yet another specific embodiment, the method of treating cancer comprises administering to a human patient in need thereof: AMP-514, GSK2813781, and MTIG7192A (RG6058).

In still another embodiment, the method of treating cancer comprises administering to a human patient in need thereof: AMP-514, GSK2813781, and PTZ-201 (ASP8374).

In one specific embodiment, the method of treating cancer comprises administering to a human patient in need thereof: PDR001, BMS-986016, and BMS-986207.

In another specific embodiment, the method of treating cancer comprises administering to a human patient in need thereof: PDR001, BMS-986016, and OMP-313M32.

In yet another specific embodiment, the method of treating cancer comprises administering to a human patient in need thereof: PDR001, BMS-986016, and MTIG7192A (RG6058).

In still another embodiment, the method of treating cancer comprises administering to a human patient in need thereof: PDR001, BMS-986016, and PTZ-201 (ASP8374).

In one specific embodiment, the method of treating cancer comprises administering to a human patient in need thereof: PDR001, REGN3767, and BMS-986207.

In another specific embodiment, the method of treating cancer comprises administering to a human patient in need thereof: PDR001, REGN3767, and OMP-313M32.

In yet another specific embodiment, the method of treating cancer comprises administering to a human patient in need thereof: PDR001, REGN3767, and MTIG7192A (RG6058).

In still another embodiment, the method of treating cancer comprises administering to a human patient in need thereof: PDR001, REGN3767, and PTZ-201 (ASP8374).

In one specific embodiment, the method of treating cancer comprises administering to a human patient in need thereof: PDR001, LAG525, and BMS-986207.

In another specific embodiment, the method of treating cancer comprises administering to a human patient in need thereof: PDR001, LAG525, and OMP-313M32.

In yet another specific embodiment, the method of treating cancer comprises administering to a human patient in need thereof: PDR001, LAG525, and MTIG7192A (RG6058).

In still another embodiment, the method of treating cancer comprises administering to a human patient in need thereof: PDR001, LAG525, and PTZ-201 (ASP8374).

In one specific embodiment, the method of treating cancer comprises administering to a human patient in need thereof: PDR001, GSK2813781, and BMS-986207.

In another specific embodiment, the method of treating cancer comprises administering to a human patient in need thereof: PDR001, GSK2813781, and OMP-313M32.

In yet another specific embodiment, the method of treating cancer comprises administering to a human patient in need thereof: PDR001, GSK2813781, and MTIG7192A (RG6058).

In still another embodiment, the method of treating cancer comprises administering to a human patient in need thereof: PDR001, GSK2813781, and PTZ-201 (ASP8374).

In one specific embodiment, the method of treating cancer comprises administering to a human patient in need thereof: BGB-A317, BMS-986016, and BMS-986207.

In another specific embodiment, the method of treating cancer comprises administering to a human patient in need thereof: BGB-A317, BMS-986016, and OMP-313M32.

In yet another specific embodiment, the method of treating cancer comprises administering to a human patient in need thereof: BGB-A317, BMS-986016, and MTIG7192A (RG6058).

In still another embodiment, the method of treating cancer comprises administering to a human patient in need thereof: BGB-A317, BMS-986016, and PTZ-201 (ASP8374).

In one specific embodiment, the method of treating cancer comprises administering to a human patient in need thereof: BGB-A317, REGN3767, and BMS-986207.

In another specific embodiment, the method of treating cancer comprises administering to a human patient in need thereof: BGB-A317, REGN3767, and OMP-313M32.

In yet another specific embodiment, the method of treating cancer comprises administering to a human patient in need thereof: BGB-A317, REGN3767, and MTIG7192A (RG6058).

In still another embodiment, the method of treating cancer comprises administering to a human patient in need thereof: BGB-A317, REGN3767, and PTZ-201 (ASP8374).

In one specific embodiment, the method of treating cancer comprises administering to a human patient in need thereof: BGB-A317, LAG525, and BMS-986207.

In another specific embodiment, the method of treating cancer comprises administering to a human patient in need thereof: BGB-A317, LAG525, and OMP-313M32.

In yet another specific embodiment, the method of treating cancer comprises administering to a human patient in need thereof: BGB-A317, LAG525, and MTIG7192A (RG6058).

In still another embodiment, the method of treating cancer comprises administering to a human patient in need thereof: BGB-A317, LAG525, and PTZ-201 (ASP8374).

In one specific embodiment, the method of treating cancer comprises administering to a human patient in need thereof: BGB-A317, GSK2813781, and BMS-986207.

In another specific embodiment, the method of treating cancer comprises administering to a human patient in need thereof: BGB-A317, GSK2813781, and OMP-313M32.

In yet another specific embodiment, the method of treating cancer comprises administering to a human patient in need thereof: BGB-A317, GSK2813781, and MTIG7192A (RG6058).

In still another embodiment, the method of treating cancer comprises administering to a human patient in need thereof: BGB-A317, GSK2813781, and PTZ-201 (ASP8374).

In one specific embodiment, the method of treating cancer comprises administering to a human patient in need thereof: MGA012, BMS-986016, and BMS-986207.

In another specific embodiment, the method of treating cancer comprises administering to a human patient in need thereof: MGA012, BMS-986016, and OMP-313M32.

In yet another specific embodiment, the method of treating cancer comprises administering to a human patient in need thereof: MGA012, BMS-986016, and MTIG7192A (RG6058).

In still another embodiment, the method of treating cancer comprises administering to a human patient in need thereof: MGA012, BMS-986016, and PTZ-201 (ASP8374).

In one specific embodiment, the method of treating cancer comprises administering to a human patient in need thereof: MGA012, REGN3767, and BMS-986207.

In another specific embodiment, the method of treating cancer comprises administering to a human patient in need thereof: MGA012, REGN3767, and OMP-313M32.

In yet another specific embodiment, the method of treating cancer comprises administering to a human patient in need thereof: MGA012, REGN3767, and MTIG7192A (RG6058).

In still another embodiment, the method of treating cancer comprises administering to a human patient in need thereof: MGA012, REGN3767, and PTZ-201 (ASP8374).

In one specific embodiment, the method of treating cancer comprises administering to a human patient in need thereof: MGA012, LAG525, and BMS-986207.

In another specific embodiment, the method of treating cancer comprises administering to a human patient in need thereof: MGA012, LAG525, and OMP-313M32.

In yet another specific embodiment, the method of treating cancer comprises administering to a human patient in need thereof: MGA012, LAG525, and MTIG7192A (RG6058).

In still another embodiment, the method of treating cancer comprises administering to a human patient in need thereof: MGA012, LAG525, and PTZ-201 (ASP8374).

In one specific embodiment, the method of treating cancer comprises administering to a human patient in need thereof: MGA012, GSK2813781, and BMS-986207.

In another specific embodiment, the method of treating cancer comprises administering to a human patient in need thereof: MGA012, GSK2813781, and OMP-313M32.

In yet another specific embodiment, the method of treating cancer comprises administering to a human patient in need thereof: MGA012, GSK2813781, and MTIG7192A (RG6058).

In still another embodiment, the method of treating cancer comprises administering to a human patient in need thereof: MGA012, GSK2813781, and PTZ-201 (ASP8374).

In certain embodiments, provided herein is a method of treating colorectal cancer, comprising administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In some embodiments, provided herein is a method of treating gastric cancer, comprising administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In other embodiments, provided herein is a method of treating head and neck cancer, comprising administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In yet other embodiments, provided herein is a method of treating refractory head and neck cancer, comprising administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In still other embodiments, provided herein is a method of treating lung cancer, comprising administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In certain embodiments, provided herein is a method of treating non-small cell lung cancer, comprising administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In some embodiments, provided herein is a method of treating breast cancer, comprising administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In other embodiments, provided herein is a method of treating cervical cancer, comprising administering to a human patient in need thereof(a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In yet other embodiments, provided herein is a method of treating ovarian cancer, comprising administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In still other embodiments, provided herein is a method of treating DLBCL, comprising administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In certain embodiments, provided herein is a method of treating NHL, comprising administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In some embodiments, provided herein is a method of treating rrNHL, comprising administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In other embodiments, provided herein is a method of treating naïve rrNHL, comprising administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In yet other embodiments, provided herein is a method of treating PD-1 refractory rrNHL, comprising administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In certain embodiments, provided herein is a method of treating colorectal cancer, comprising administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In some embodiments, provided herein is a method of treating gastric cancer, comprising administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In other embodiments, provided herein is a method of treating head and neck cancer, comprising administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In yet other embodiments, provided herein is a method of treating refractory head and neck cancer, comprising administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In still other embodiments, provided herein is a method of treating lung cancer, comprising administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In certain embodiments, provided herein is a method of treating non-small cell lung cancer, comprising administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In some embodiments, provided herein is a method of treating breast cancer, comprising administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In other embodiments, provided herein is a method of treating cervical cancer, comprising administering to a human patient in need thereof(a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In yet other embodiments, provided herein is a method of treating ovarian cancer, comprising administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In still other embodiments, provided herein is a method of treating DLBCL, comprising administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In certain embodiments, provided herein is a method of treating NHL, comprising administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In some embodiments, provided herein is a method of treating rrNHL, comprising administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In other embodiments, provided herein is a method of treating naïve rrNHL, comprising administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In yet other embodiments, provided herein is a method of treating PD-1 refractory rrNHL, comprising administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In some embodiments, the anti-human PD-1 monoclonal antibody is a human antibody. In other embodiments, the anti-human PD-1 monoclonal antibody is a humanized antibody.

In some embodiments, the anti-human LAG3 monoclonal antibody is a human antibody. In other embodiments, the anti-human LAG3 monoclonal antibody is a humanized antibody.

In some embodiments, the anti-human TIGIT monoclonal antibody is a human antibody. In other embodiments, the anti-human TIGIT monoclonal antibody is a humanized antibody.

Thus, in certain embodiments, provided herein is a method for treating cancer, comprising administering to a human patient in need thereof:
(a) a human or humanized anti-human PD-1 monoclonal antibody or antigen binding fragment thereof;
(b) a human or humanized anti-human LAG3 monoclonal antibody or antigen binding fragment thereof; and
(c) a human or humanized anti-human TIGIT monoclonal antibody or antigen binding fragment thereof.

In some embodiments, provided herein is a method for treating cancer, comprising administering to a human patient in need thereof:
(a) a human anti-human PD-1 monoclonal antibody or antigen binding fragment thereof;
(b) a human anti-human LAG3 monoclonal antibody or antigen binding fragment thereof; and
(c) a human anti-human TIGIT monoclonal antibody or antigen binding fragment thereof.

In other embodiments, provided herein is a method for treating cancer, comprising administering to a human patient in need thereof:
(a) a humanized anti-human PD-1 monoclonal antibody or antigen binding fragment thereof;
(b) a humanized anti-human LAG3 monoclonal antibody or antigen binding fragment thereof; and
(c) a humanized anti-human TIGIT monoclonal antibody or antigen binding fragment thereof.

Also provided herein are methods for treating an infectious disease, comprising administering to a human patient in need thereof:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In one embodiment, the infectious disease is viral infection. In another embodiment, the infectious disease is bacterial infection. In yet another embodiment, the infectious disease is parasitic infection. In still another embodiment, the infectious disease is fungal infection.

In certain embodiments, the viral infection is infection with a virus selected from the group consisting of human immunodeficiency virus (HIV), ebola virus, hepatitis virus A (HAV), hepatitis virus B (HBV), hepatitis virus C (HCV), herpes virus (e.g., VZV, HSV-I, HAV-6, HSV-II, CMV, Epstein Barr virus), adenovirus, influenza virus, flavivirus, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus, and arboviral encephalitis virus.

In some embodiments, the bacterial infection is infection with a bacteria selected from the group consisting of chlamydia, rickettsia, mycobacteria, staphylococci, streptococci, pneumonococci, meningococci, gonococci, klebsiella, proteus, serratia, pseudomonas, legionella, salmonella, bacilli, borriella, Corynebacterium diphtheriae, Vibrio cholerae, Clostridium tetan, Clostridium botulinum, Bacillus anthricis, Yersinia pestis, Mycobacterium leprae, and Mycobacterium lepromatosis.

In other embodiments, the fungal infection is infection with a fungus selected from the group consisting of Candida (albicans, krusei, glabrata, tropicalis, etc.), Cryptococcus neoformans, Aspergillus (fumigatus, niger, etc.), Genus Mucorales (mucor, absidia, rhizopus), Sporothrix schenkii, Blastomyces dermatitidis, Paracoccidioides brasiliensis, Coccidioides immitis and Histoplasma capsulatum.

In yet other embodiments, the parasitic infection is infection with a parasite selected from the group consisting of Entamoeba histolytica, Balantidium coli, Naegleria fowleri, Acanthamoeba, Giardia lambia, Cryptosporidium, Pneumocystis carinii, Plasmodium vivax, Babesia microti, Trypanosoma brucei, Trypanosoma cruzi, Leishmania donovani, Toxoplasma gondii, Nippostrongylus brasiliensis.

In some embodiments, the method for treating an infectious disease comprises administering to a human patient in need thereof:
(a) an anti-human PD-1 monoclonal antibody or antigen binding fragment thereof disclosed in Section V.3;
(b) an anti-human LAG3 monoclonal antibody or antigen binding fragment thereof disclosed in Section V.4; and
(c) an anti-human TIGIT monoclonal antibody or antigen binding fragment thereof disclosed in Section V.5.

In one specific embodiment, the method of treating an infectious disease comprises contacting the T cells with: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In another specific embodiment, the method of treating an infectious disease comprises administering to a human patient in need thereof: (a) an anti-human PD-1 monoclonal antibody or antigen binding fragment thereof that comprises a V_{L} region comprising an amino acid sequence as set forth in SEQ ID NO:4, and a V_{H} region comprising an amino acid sequence as set forth in SEQ ID NO:9; (b) an anti-human LAG3 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In yet another specific embodiment, the method of treating an infectious disease comprises administering to a human patient in need thereof: (a) an anti-human PD-1 monoclonal antibody or antigen binding fragment thereof that comprises a light chain comprising or consisting of an amino acid sequence as set forth in SEQ ID NO:5 and a heavy chain comprising or consisting of an amino acid sequence as set forth in SEQ ID NO:10; (b) an anti-human LAG3 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In one specific embodiment, the method of treating an infectious disease comprises administering to a human patient in need thereof: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In another specific embodiment, the method of treating an infectious disease comprises administering to a human patient in need thereof: (a) an anti-human PD-1 monoclonal antibody or antigen binding fragment thereof that comprises a V_{L} region comprising an amino acid sequence as set forth in SEQ ID NO:34, and a V_{H} region comprising an amino acid sequence as set forth in SEQ ID NO:39; (b) an anti-human LAG3 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In yet another specific embodiment, the method of treating an infectious disease comprises administering to a human patient in need thereof: (a) an anti-human PD-1 monoclonal antibody or antigen binding fragment thereof that comprises a light chain comprising or consisting of an amino acid sequence as set forth in SEQ ID NO:35 and a heavy chain comprising or consisting of an amino acid sequence as set forth in SEQ ID NO:40; (b) an anti-human LAG3 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In some embodiments, the anti-human PD-1 monoclonal antibody is a human antibody. In other embodiments, the anti-human PD-1 monoclonal antibody is a humanized antibody.

In some embodiments, the anti-human LAG3 monoclonal antibody is a human antibody. In other embodiments, the anti-human LAG3 monoclonal antibody is a humanized antibody.

In some embodiments, the anti-human TIGIT monoclonal antibody is a human antibody. In other embodiments, the anti-human TIGIT monoclonal antibody is a humanized antibody.

Thus, in certain embodiments, provided herein is a method for treating an infectious disease, comprising administering to a human patient in need thereof:
(a) a human or humanized anti-human PD-1 monoclonal antibody or antigen binding fragment thereof;
(b) a human or humanized anti-human LAG3 monoclonal antibody or antigen binding fragment thereof; and
(c) a human or humanized anti-human TIGIT monoclonal antibody or antigen binding fragment thereof.

In some embodiments, provided herein is a method for treating an infectious disease, comprising administering to a human patient in need thereof:
(a) a human anti-human PD-1 monoclonal antibody or antigen binding fragment thereof;
(b) a human anti-human LAG3 monoclonal antibody or antigen binding fragment thereof; and
(c) a human anti-human TIGIT monoclonal antibody or antigen binding fragment thereof.

In other embodiments, provided herein is a method for treating an infectious disease, comprising administering to a human patient in need thereof:
(a) a humanized anti-human PD-1 monoclonal antibody or antigen binding fragment thereof;
(b) a humanized anti-human LAG3 monoclonal antibody or antigen binding fragment thereof; and
(c) a humanized anti-human TIGIT monoclonal antibody or antigen binding fragment thereof.

Any combination of an anti-PD-1 monoclonal antibody or antigen binding fragment thereof disclosed in Section V.3, an anti-LAG3 monoclonal antibody or antigen binding fragment thereof disclosed in Section V.4, and an anti-TIGIT monoclonal antibody or antigen binding fragment thereof disclosed in Section V.5 are contemplated herein in methods of treating cancer (e.g., colorectal cancer) or an infectious disease (e.g., a viral infection, a bacterial infection, etc.).

7. Methods of Enhancing Immune Cell Activity Using a Combination of an Anti-PD-1 Monoclonal Antibody, an Anti-LAG3 Monoclonal Antibody, and an Anti-TIGIT Monoclonal Antibody

Further, the antibodies or antigen binding fragments of disclosed herein can enhance the activity of an immune cell. The increase of the activity of an immune cell can be detected using any methods known in the art. In one embodiment, the increase in activity of an immune cell can be detected by measuring the proliferation of the immune cell. For example, an increase in activity of a T cell can be detected by measuring the proliferation of the T cell or signal transduction events such as tyrosine phosphorylation of immune receptors or downstream kinases that transmit signals to transcriptional regulators. In other embodiments, the increase in activity of an immune cell can be detected by measuring CTL or NK cell cytotoxic function on specific target cells or IFN-γ cytokine responses, which are associated with stimulation of immunity. In yet other embodiments, the increase in activity of an immune cell can be detected by measuring T cell activation ex vivo in a sample derived from the subject. In one embodiment, the increase in T cell activity is determined by measuring production of one or more cytokines selected from the group consisting of: IL-1, IL-2, IL-6, IL-12, IL-17, IL-22, IL-23, GM-CSF, IFN-γ, and TNF-α. In some embodiments, the ability of the antibodies or antigen binding fragments of the invention to increase the activity of an immune cell can be detected by CD25 and CD69 upregulation by flow cytometry.

Thus, also provided herein are methods of enhancing T cell activity (e.g., increasing cytokine production and/or cell proliferation), comprising contacting the T cells with:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In some embodiments, the enhancement of T cell activity occurs in vitro. In other embodiments, the enhancement of T cell activity occurs in vivo.

In some embodiments, the enhancement of T cell activity is measured by increased cytokine production. In other embodiments, the enhancement of T cell activity is measured by increased cell proliferation.

Thus, in certain embodiments, provided herein is a method of increasing cytokine production of T cells, comprising contacting the T cells with:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In some embodiments, the increased cytokine production of T cells occurs in vitro. In other embodiments, the increased cytokine production of T cells occurs in vivo.

In some embodiments, provided herein is a method of increasing proliferation of T cells, comprising contacting the T cells with:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In some embodiments, the increased proliferation of T cells occurs in vitro. In other embodiments, the increased proliferation of T cells occurs in vivo.

In other embodiments, provided herein is a method of increasing cytokine production and proliferation of T cells, comprising contacting the T cells with:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In some embodiments, the increased cytokine production and the increased proliferation of T cells occur in vitro. In other embodiments, the increased cytokine production and the increased proliferation of T cells occur in vivo.

In certain embodiments, the cytokine is selected from the group consisting of IL-1, IL-2, IL-6, IL-12, IL-17, IL-22, IL-23, GM-CSF, IFN-γ, and TNF-α. In one embodiment, the cytokine is IL-1. In another embodiment, the cytokine is IL-2. In yet another embodiment, the cytokine is IL-6. In still another embodiment, the cytokine is IL-12. In one embodiment, the cytokine is IL-17. In another embodiment, the cytokine is IL-22. In yet another embodiment, the cytokine is IL-23. In still another embodiment, the cytokine is GM-CSF. In one embodiment, the cytokine is IFN-γ. In another embodiment, the cytokine is TNF-α. In some embodiments, the cytokine is one, two, three, four, five, six, seven, eight, nine, or ten cytokines selected from the group consisting of IL-1, IL-2, IL-6, IL-12, IL-17, IL-22, IL-23, GM-CSF, IFN-γ, and TNF-α.

In some embodiments, the maximal enhancement of T cell activity is at least about 10%, 20%, 30%, 40%, 45%, 50%, 55%, 60%, 65%, 70%, 75%, 80%, 85%, 90%, 95%, 100%, 2-fold, 3-fold, 4-fold, 5-fold, 10-fold, 15-fold, 20-fold, 50-fold, 75-fold, or 100-fold. In certain embodiments, the maximal increase of cytokine production is at least about 10%, 20%, 30%, 40%, 45%, 50%, 55%, 60%, 65%, 70%, 75%, 80%, 85%, 90%, 95%, 100%, 2-fold, 3-fold, 4-fold, 5-fold, 10-fold, 15-fold, 20-fold, 50-fold, 75-fold, or 100-fold. In other embodiments, the maximal increase of T cell proliferation is at least about 10%, 20%, 30%, 40%, 45%, 50%, 55%, 60%, 65%, 70%, 75%, 80%, 85%, 90%, 95%, 100%, 2-fold, 3-fold, 4-fold, 5-fold, 10-fold, 15-fold, 20-fold, 50-fold, 75-fold, or 100-fold.

In some embodiments, the maximal enhancement of T cell activity is relative to the T cell activity in cells that are not contacted with a combination of an anti-PD-1 monoclonal antibody or antigen binding fragment thereof, an anti-LAG3 monoclonal antibody or antigen binding fragment thereof, and an anti-TIGIT monoclonal antibody or antigen binding fragment thereof. In some embodiments, the maximal enhancement of T cell activity is relative to the T cell activity in cells that are not contacted with any antibody at all. In some embodiments, the maximal enhancement of T cell activity is relative to the T cell activity in cells that are contacted with an unrelated antibody (e.g., an antibody that does not specifically bind to PD-1, LAG3, or TIGIT). In some embodiments, the maximal enhancement of T cell activity is relative to the T cell activity in cells that are contacted with one or two antibodies selected from the group consisting of an anti-PD-1 monoclonal antibody or antigen binding fragment thereof, an anti-LAG3 monoclonal antibody or antigen binding fragment thereof, and an anti-TIGIT monoclonal antibody or antigen binding fragment thereof. In some embodiments, the maximal enhancement of T cell activity is relative to the T cell activity in cells that are contacted with an anti-PD-1 monoclonal antibody or antigen binding fragment thereof. In some embodiments, the maximal enhancement of T cell activity is relative to the T cell activity in cells that are contacted with an anti-LAG3 monoclonal antibody or antigen binding fragment thereof. In some embodiments, the maximal enhancement of T cell activity is relative to the T cell activity in cells that are contacted with an anti-TIGIT monoclonal antibody or antigen binding fragment thereof. In some embodiments, the maximal enhancement of T cell activity is relative to the T cell activity in cells that are contacted with an anti-PD-1 monoclonal antibody or antigen binding fragment thereof and an anti-LAG3 monoclonal antibody or antigen binding fragment thereof. In some embodiments, the maximal enhancement of T cell activity is relative to the T cell activity in cells that are contacted with an anti-PD-1 monoclonal antibody or antigen binding fragment thereof and an anti-TIGIT monoclonal antibody or antigen binding fragment thereof. In some embodiments, the maximal enhancement of T cell activity is relative to the T cell activity in cells that are contacted with an anti-LAG3 monoclonal antibody or antigen binding fragment thereof and an anti-TIGIT monoclonal antibody or antigen binding fragment thereof.

In some embodiments, the maximal increase of cytokine production is relative to the cytokine production in cells that are not contacted with a combination of an anti-PD-1 monoclonal antibody or antigen binding fragment thereof, an anti-LAG3 monoclonal antibody or antigen binding fragment thereof, and an anti-TIGIT monoclonal antibody or antigen binding fragment thereof. In some embodiments, the maximal increase of cytokine production is relative to the cytokine production in cells that are not contacted with any antibody at all. In some embodiments, the maximal increase of cytokine production is relative to the cytokine production in cells that are contacted with an unrelated antibody (e.g., an antibody that does not specifically bind to PD-1, LAG3, or TIGIT). In some embodiments, the maximal increase of cytokine production is relative to the cytokine production in cells that are contacted with one or two antibodies selected from the group consisting of an anti-PD-1 monoclonal antibody or antigen binding fragment thereof, an anti-LAG3 monoclonal antibody or antigen binding fragment thereof, and an anti-TIGIT monoclonal antibody or antigen binding fragment thereof. In some embodiments, the maximal increase of cytokine production is relative to the cytokine production in cells that are contacted with an anti-PD-1 monoclonal antibody or antigen binding fragment thereof. In some embodiments, the maximal increase of cytokine production is relative to the cytokine production in cells that are contacted with an anti-LAG3 monoclonal antibody or antigen binding fragment thereof. In some embodiments, the maximal increase of cytokine production is relative to the cytokine production in cells that are contacted with an anti-TIGIT monoclonal antibody or antigen binding fragment thereof. In some embodiments, the maximal increase of cytokine production is relative to the cytokine production in cells that are contacted with an anti-PD-1 monoclonal antibody or antigen binding fragment thereof and an anti-LAG3 monoclonal antibody or antigen binding fragment thereof. In some embodiments, the maximal increase of cytokine production is relative to the cytokine production in cells that are contacted with an anti-PD-1 monoclonal antibody or antigen binding fragment thereof and an anti-TIGIT monoclonal antibody or antigen binding fragment thereof. In some embodiments, the maximal increase of cytokine production is relative to the cytokine production in cells that are contacted with an anti-LAG3 monoclonal antibody or antigen binding fragment thereof and an anti-TIGIT monoclonal antibody or antigen binding fragment thereof.

In some embodiments, the maximal increase of T cell proliferation is relative to the proliferation of T cells that are not contacted with a combination of an anti-PD-1 monoclonal antibody or antigen binding fragment thereof, an anti-LAG3 monoclonal antibody or antigen binding fragment thereof, and an anti-TIGIT monoclonal antibody or antigen binding fragment thereof. In some embodiments, the maximal increase of T cell proliferation is relative to the proliferation of T cells that are not contacted with any antibody at all. In some embodiments, the maximal increase of T cell proliferation is relative to the proliferation of T cells that are contacted with an unrelated antibody (e.g., an antibody that does not specifically bind to PD-1, LAG3, or TIGIT). In some embodiments, the maximal increase of T cell proliferation is relative to the proliferation of T cells that are contacted with one or two antibodies selected from the group consisting of an anti-PD-1 monoclonal antibody or antigen binding fragment thereof, an anti-LAG3 monoclonal antibody or antigen binding fragment thereof, and an anti-TIGIT monoclonal antibody or antigen binding fragment thereof. In some embodiments, the maximal increase of T cell proliferation is relative to the proliferation of T cells that are contacted with an anti-PD-1 monoclonal antibody or antigen binding fragment thereof. In some embodiments, the maximal increase of T cell proliferation is relative to the proliferation of T cells that are contacted with an anti-LAG3 monoclonal antibody or antigen binding fragment thereof. In some embodiments, the maximal increase of T cell proliferation is relative to the proliferation of T cells that are contacted with an anti-TIGIT monoclonal antibody or antigen binding fragment thereof. In some embodiments, the maximal increase of T cell proliferation is relative to the proliferation of T cells that are contacted with an anti-PD-1 monoclonal antibody or antigen binding fragment thereof and an anti-LAG3 monoclonal antibody or antigen binding fragment thereof. In some embodiments, the maximal increase of T cell proliferation is relative to the proliferation of T cells that are contacted with an anti-PD-1 monoclonal antibody or antigen binding fragment thereof and an anti-TIGIT monoclonal antibody or antigen binding fragment thereof. In some embodiments, the maximal increase of T cell proliferation is relative to the proliferation of T cells that are contacted with an anti-LAG3 monoclonal antibody or antigen binding fragment thereof and an anti-TIGIT monoclonal antibody or antigen binding fragment thereof.

In some embodiments, the EC₅₀ value of cytokine production is at most about 50, 40, 30, 20, 10, 5, 1, 0.75, 0.5, 0.1, 0.05, 0.01, 0.005, or 0.001 nM. In certain embodiments, the EC₅₀ value of cytokine production is at least about 50, 40, 30, 20, 10, 5, 1, 0.75, 0.5, 0.1, 0.05, 0.01, 0.005, or 0.001 nM. In other embodiments, the EC₅₀ value of T cell proliferation is at most about 50, 40, 30, 20, 10, 5, 1, 0.75, 0.5, 0.1, 0.05, 0.01, 0.005, or 0.001 nM. In yet other embodiments, the EC₅₀ value of T cell proliferation is at least about 50, 40, 30, 20, 10, 5, 1, 0.75, 0.5, 0.1, 0.05, 0.01, 0.005, or 0.001 nM.

In certain embodiments, the maximal increase of IL-1 production is at least about 10%, 20%, 30%, 40%, 45%, 50%, 55%, 60%, 65%, 70%, 75%, 80%, 85%, 90%, 95%, 100%, 2-fold, 3-fold, 4-fold, 5-fold, 10-fold, 15-fold, 20-fold, 50-fold, 75-fold, or 100-fold. In some embodiments, the EC₅₀ value of IL-1 production is at most about 50, 40, 30, 20, 10, 5, 1, 0.75, 0.5, 0.1, 0.05, 0.01, 0.005, or 0.001 nM. In certain embodiments, the EC₅₀ value of IL-1 production is at least about 50, 40, 30, 20, 10, 5, 1, 0.75, 0.5, 0.1, 0.05, 0.01, 0.005, or 0.001 nM.

In certain embodiments, the maximal increase of IL-2 production is at least about 10%, 20%, 30%, 40%, 45%, 50%, 55%, 60%, 65%, 70%, 75%, 80%, 85%, 90%, 95%, 100%, 2-fold, 3-fold, 4-fold, 5-fold, 10-fold, 15-fold, 20-fold, 50-fold, 75-fold, or 100-fold. In some embodiments, the EC₅₀ value of IL-2 production is at most about 50, 40, 30, 20, 10, 5, 1, 0.75, 0.5, 0.1, 0.05, 0.01, 0.005, or 0.001 nM. In certain embodiments, the EC₅₀ value of IL-2 production is at least about 50, 40, 30, 20, 10, 5, 1, 0.75, 0.5, 0.1, 0.05, 0.01, 0.005, or 0.001 nM.

In certain embodiments, the maximal increase of IL-6 production is at least about 10%, 20%, 30%, 40%, 45%, 50%, 55%, 60%, 65%, 70%, 75%, 80%, 85%, 90%, 95%, 100%, 2-fold, 3-fold, 4-fold, 5-fold, 10-fold, 15-fold, 20-fold, 50-fold, 75-fold, or 100-fold. In some embodiments, the EC₅₀ value of IL-6 production is at most about 50, 40, 30, 20, 10, 5, 1, 0.75, 0.5, 0.1, 0.05, 0.01, 0.005, or 0.001 nM. In certain embodiments, the EC₅₀ value of IL-6 production is at least about 50, 40, 30, 20, 10, 5, 1, 0.75, 0.5, 0.1, 0.05, 0.01, 0.005, or 0.001 nM.

In certain embodiments, the maximal increase of IL-12 production is at least about 10%, 20%, 30%, 40%, 45%, 50%, 55%, 60%, 65%, 70%, 75%, 80%, 85%, 90%, 95%, 100%, 2-fold, 3-fold, 4-fold, 5-fold, 10-fold, 15-fold, 20-fold, 50-fold, 75-fold, or 100-fold. In some embodiments, the EC₅₀ value of IL-12 production is at most about 50, 40, 30, 20, 10, 5, 1, 0.75, 0.5, 0.1, 0.05, 0.01, 0.005, or 0.001 nM. In certain embodiments, the EC₅₀ value of IL-12 production is at least about 50, 40, 30, 20, 10, 5, 1, 0.75, 0.5, 0.1, 0.05, 0.01, 0.005, or 0.001 nM.

In certain embodiments, the maximal increase of IL-17 production is at least about 10%, 20%, 30%, 40%, 45%, 50%, 55%, 60%, 65%, 70%, 75%, 80%, 85%, 90%, 95%, 100%, 2-fold, 3-fold, 4-fold, 5-fold, 10-fold, 15-fold, 20-fold, 50-fold, 75-fold, or 100-fold. In some embodiments, the EC₅₀ value of IL-17 production is at most about 50, 40, 30, 20, 10, 5, 1, 0.75, 0.5, 0.1, 0.05, 0.01, 0.005, or 0.001 nM. In certain embodiments, the EC₅₀ value of IL-17 production is at least about 50, 40, 30, 20, 10, 5, 1, 0.75, 0.5, 0.1, 0.05, 0.01, 0.005, or 0.001 nM.

In certain embodiments, the maximal increase of IL-22 production is at least about 10%, 20%, 30%, 40%, 45%, 50%, 55%, 60%, 65%, 70%, 75%, 80%, 85%, 90%, 95%, 100%, 2-fold, 3-fold, 4-fold, 5-fold, 10-fold, 15-fold, 20-fold, 50-fold, 75-fold, or 100-fold. In some embodiments, the EC₅₀ value of IL-22 production is at most about 50, 40, 30, 20, 10, 5, 1, 0.75, 0.5, 0.1, 0.05, 0.01, 0.005, or 0.001 nM. In certain embodiments, the EC₅₀ value of IL-22 production is at least about 50, 40, 30, 20, 10, 5, 1, 0.75, 0.5, 0.1, 0.05, 0.01, 0.005, or 0.001 nM.

In certain embodiments, the maximal increase of IL-23 production is at least about 10%, 20%, 30%, 40%, 45%, 50%, 55%, 60%, 65%, 70%, 75%, 80%, 85%, 90%, 95%, 100%, 2-fold, 3-fold, 4-fold, 5-fold, 10-fold, 15-fold, 20-fold, 50-fold, 75-fold, or 100-fold. In some embodiments, the EC₅₀ value of IL-23 production is at most about 50, 40, 30, 20, 10, 5, 1, 0.75, 0.5, 0.1, 0.05, 0.01, 0.005, or 0.001 nM. In certain embodiments, the EC₅₀ value of IL-23 production is at least about 50, 40, 30, 20, 10, 5, 1, 0.75, 0.5, 0.1, 0.05, 0.01, 0.005, or 0.001 nM.

In certain embodiments, the maximal increase of GM-CSF production is at least about 10%, 20%, 30%, 40%, 45%, 50%, 55%, 60%, 65%, 70%, 75%, 80%, 85%, 90%, 95%, 100%, 2-fold, 3-fold, 4-fold, 5-fold, 10-fold, 15-fold, 20-fold, 50-fold, 75-fold, or 100-fold. In some embodiments, the EC₅₀ value of GM-CSF production is at most about 50, 40, 30, 20, 10, 5, 1, 0.75, 0.5, 0.1, 0.05, 0.01, 0.005, or 0.001 nM. In certain embodiments, the EC₅₀ value of GM-CSF production is at least about 50, 40, 30, 20, 10, 5, 1, 0.75, 0.5, 0.1, 0.05, 0.01, 0.005, or 0.001 nM.

In certain embodiments, the maximal increase of IFN-γ production is at least about 10%, 20%, 30%, 40%, 45%, 50%, 55%, 60%, 65%, 70%, 75%, 80%, 85%, 90%, 95%, 100%, 2-fold, 3-fold, 4-fold, 5-fold, 10-fold, 15-fold, 20-fold, 50-fold, 75-fold, or 100-fold. In some embodiments, the EC₅₀ value of IFN-γ production is at most about 50, 40, 30, 20, 10, 5, 1, 0.75, 0.5, 0.1, 0.05, 0.01, 0.005, or 0.001 nM. In certain embodiments, the EC₅₀ value of IFN-γ production is at least about 50, 40, 30, 20, 10, 5, 1, 0.75, 0.5, 0.1, 0.05, 0.01, 0.005, or 0.001 nM.

In certain embodiments, the maximal increase of TNF-α production is at least about 10%, 20%, 30%, 40%, 45%, 50%, 55%, 60%, 65%, 70%, 75%, 80%, 85%, 90%, 95%, 100%, 2-fold, 3-fold, 4-fold, 5-fold, 10-fold, 15-fold, 20-fold, 50-fold, 75-fold, or 100-fold. In some embodiments, the EC₅₀ value of TNF-α production is at most about 50, 40, 30, 20, 10, 5, 1, 0.75, 0.5, 0.1, 0.05, 0.01, 0.005, or 0.001 nM. In certain embodiments, the EC₅₀ value of TNF-α production is at least about 50, 40, 30, 20, 10, 5, 1, 0.75, 0.5, 0.1, 0.05, 0.01, 0.005, or 0.001 nM.

In one embodiment, the maximal increase of IFN-γ production is at least about 10%. In another embodiment, the maximal increase of IFN-γ production is at least about 20%.

In yet another embodiment, the maximal increase of IFN-γ production is at least about 30%. In still another embodiment, the maximal increase of IFN-γ production is at least about 40%. In one embodiment, the maximal increase of IFN-γ production is at least about 50%. In another embodiment, the maximal increase of IFN-γ production is at least about 60%. In yet another embodiment, the maximal increase of IFN-γ production is at least about 70%. In still another embodiment, the maximal increase of IFN-γ production is at least about 80%. In one embodiment, the maximal increase of IFN-γ production is at least about 90%. In another embodiment, the maximal increase of IFN-γ production is at least about 100%. In yet another embodiment, the maximal increase of IFN-γ production is at least about 2-fold. In still another embodiment, the maximal increase of IFN-γ production is at least about 3-fold. In one embodiment, the maximal increase of IFN-γ production is at least about 4-fold. In another embodiment, the maximal increase of IFN-γ production is at least about 5-fold. In yet another embodiment, the maximal increase of IFN-γ production is at least about 10-fold. In still another embodiment, the maximal increase of IFN-γ production is at least about 50-fold. In one embodiment, the maximal increase of IFN-γ production is at least about 100-fold.

In one embodiment, the EC₅₀ value of IFN-γ production is at most about 50 nM. In another embodiment, the EC₅₀ value of IFN-γ production is at most about 40 nM. In yet another embodiment, the EC₅₀ value of IFN-γ production is at most about 30 nM. In still another embodiment, the EC₅₀ value of IFN-γ production is at most about 20 nM. In one embodiment, the EC₅₀ value of IFN-γ production is at most about 10 nM. In another embodiment, the EC₅₀ value of IFN-γ production is at most about 5 nM. In yet another embodiment, the EC₅₀ value of IFN-γ production is at most about 1 nM. In still another embodiment, the EC₅₀ value of IFN-γ production is at most about 0.75 nM. In one embodiment, the EC₅₀ value of IFN-γ production is at most about 0.5 nM. In another embodiment, the EC₅₀ value of IFN-γ production is at most about 0.1 nM. In yet another embodiment, the EC₅₀ value of IFN-γ production is at most about 0.05 nM. In still another embodiment, the EC₅₀ value of IFN-γ production is at most about 0.01 nM. In one embodiment, the EC₅₀ value of IFN-γ production is at most about 0.005 nM. In another embodiment, EC₅₀ value of IFN-γ production is at most about 0.001 nM.

In one embodiment, the EC₅₀ value of IFN-γ production is at least about 50 nM. In another embodiment, the EC₅₀ value of IFN-γ production is at least about 40 nM. In yet another embodiment, the EC₅₀ value of IFN-γ production is at least about 30 nM. In still another embodiment, the EC₅₀ value of IFN-γ production is at least about 20 nM. In one embodiment, the EC₅₀ value of IFN-γ production is at least about 10 nM. In another embodiment, the EC₅₀ value of IFN-γ production is at least about 5 nM. In yet another embodiment, the EC₅₀ value of IFN-γ production is at least about 1 nM. In still another embodiment, the EC₅₀ value of IFN-γ production is at least about 0.75 nM. In one embodiment, the EC₅₀ value of IFN-γ production is at least about 0.5 nM. In another embodiment, the EC₅₀ value of IFN-γ production is at least about 0.1 nM. In yet another embodiment, the EC₅₀ value of IFN-γ production is at least about 0.05 nM. In still another embodiment, the EC₅₀ value of IFN-γ production is at least about 0.01 nM. In one embodiment, the EC₅₀ value of IFN-γ production is at least about 0.005 nM. In another embodiment, EC₅₀ value of IFN-γ production is at least about 0.001 nM.

In one embodiment, the maximal increase of T cell proliferation is at least about 10%. In another embodiment, the maximal increase of T cell proliferation is at least about 20%. In yet another embodiment, the maximal increase of T cell proliferation is at least about 30%. In still another embodiment, the maximal increase of T cell proliferation is at least about 40%. In one embodiment, the maximal increase of T cell proliferation is at least about 50%. In another embodiment, the maximal increase of T cell proliferation is at least about 60%. In yet another embodiment, the maximal increase of T cell proliferation is at least about 70%. In still another embodiment, the maximal increase of T cell proliferation is at least about 80%. In one embodiment, the maximal increase of T cell proliferation is at least about 90%. In another embodiment, the maximal increase of T cell proliferation is at least about 100%. In yet another embodiment, the maximal increase of T cell proliferation is at least about 2-fold. In still another embodiment, the maximal increase of T cell proliferation is at least about 3-fold. In one embodiment, the maximal increase of T cell proliferation is at least about 4-fold. In another embodiment, the maximal increase of T cell proliferation is at least about 5-fold. In yet another embodiment, the maximal increase of T cell proliferation is at least about 10-fold. In still another embodiment, the maximal increase of T cell proliferation is at least about 50-fold. In one embodiment, the maximal increase of T cell proliferation is at least about 100-fold.

In one embodiment, the EC₅₀ value of T cell proliferation is at most about 50 nM. In another embodiment, the EC₅₀ value of T cell proliferation is at most about 40 nM. In yet another embodiment, the EC₅₀ value of T cell proliferation is at most about 30 nM. In still another embodiment, the EC₅₀ value of T cell proliferation is at most about 20 nM. In one embodiment, the EC₅₀ value of T cell proliferation is at most about 10 nM. In another embodiment, the EC₅₀ value of T cell proliferation is at most about 5 nM. In yet another embodiment, the EC₅₀ value of T cell proliferation is at most about 1 nM. In still another embodiment, the EC₅₀ value of T cell proliferation is at most about 0.75 nM. In one embodiment, the EC₅₀ value of T cell proliferation is at most about 0.5 nM. In another embodiment, the EC₅₀ value of T cell proliferation is at most about 0.1 nM. In yet another embodiment, the EC₅₀ value of T cell proliferation is at most about 0.05 nM. In still another embodiment, the EC₅₀ value of T cell proliferation is at most about 0.01 nM. In one embodiment, the EC₅₀ value of T cell proliferation is at most about 0.005 nM. In another embodiment, EC₅₀ value of T cell proliferation is at most about 0.001 nM.

In one embodiment, the EC₅₀ value of T cell proliferation is at least about 50 nM. In another embodiment, the EC₅₀ value of T cell proliferation is at least about 40 nM. In yet another embodiment, the EC₅₀ value of T cell proliferation is at least about 30 nM. In still another embodiment, the EC₅₀ value of T cell proliferation is at least about 20 nM. In one embodiment, the EC₅₀ value of T cell proliferation is at least about 10 nM. In another embodiment, the EC₅₀ value of T cell proliferation is at least about 5 nM. In yet another embodiment, the EC₅₀ value of T cell proliferation is at least about 1 nM. In still another embodiment, the EC₅₀ value of T cell proliferation is at least about 0.75 nM. In one embodiment, the EC₅₀ value of T cell proliferation is at least about 0.5 nM. In another embodiment, the EC₅₀ value of T cell proliferation is at least about 0.1 nM. In yet another embodiment, the EC₅₀ value of T cell proliferation is at least about 0.05 nM. In still another embodiment, the EC₅₀ value of T cell proliferation is at least about 0.01 nM. In one embodiment, the EC₅₀ value of T cell proliferation is at least about 0.005 nM. In another embodiment, EC₅₀ value of T cell proliferation is at least about 0.001 nM.

In certain embodiments, the method of enhancing T cell activity comprises contacting the T cells with:
(a) an anti-human PD-1 monoclonal antibody or antigen binding fragment thereof as disclosed in Section V.3;
(b) an anti-human LAG3 monoclonal antibody or antigen binding fragment thereof as disclosed in Section V.4; and
(c) an anti-human TIGIT monoclonal antibody or antigen binding fragment thereof as disclosed in Section V.5.

In some embodiments, the enhancement of T cell activity occurs in vitro. In other embodiments, the enhancement of T cell activity occurs in vivo.

In certain embodiments, the method of increasing cytokine production of T cells comprises contacting the T cells with:
(a) an anti-human PD-1 monoclonal antibody or antigen binding fragment thereof as disclosed in Section V.3;
(b) an anti-human LAG3 monoclonal antibody or antigen binding fragment thereof as disclosed in Section V.4; and
(c) an anti-human TIGIT monoclonal antibody or antigen binding fragment thereof as disclosed in Section V.5.

In some embodiments, the increased cytokine production of T cells occurs in vitro. In other embodiments, the increased cytokine production of T cells occurs in vivo.

In certain embodiments, the method of increasing T cell proliferation comprises contacting the T cells with:
(a) an anti-human PD-1 monoclonal antibody or antigen binding fragment thereof as disclosed in Section V.3;
(b) an anti-human LAG3 monoclonal antibody or antigen binding fragment thereof as disclosed in Section V.4; and
(c) an anti-human TIGIT monoclonal antibody or antigen binding fragment thereof as disclosed in Section V.5.

In some embodiments, the increased proliferation of T cells occurs in vitro. In other embodiments, the increased proliferation of T cells occurs in vivo.

In one specific embodiment, the method of enhancing T cell activity comprises contacting the T cells with: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of increasing cytokine production of T cells comprises contacting the T cells with: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of increasing T cell proliferation comprises contacting the T cells with: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In another specific embodiment, the method of enhancing T cell activity comprises contacting the T cells with: (a) an anti-human PD-1 monoclonal antibody or antigen binding fragment thereof that comprises a V_{L} region comprising an amino acid sequence as set forth in SEQ ID NO:4, and a V_{H} region comprising an amino acid sequence as set forth in SEQ ID NO:9; (b) an anti-human LAG3 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In another specific embodiment, the method of increasing cytokine production of T cells comprises contacting the T cells with: (a) an anti-human PD-1 monoclonal antibody or antigen binding fragment thereof that comprises a V_{L} region comprising an amino acid sequence as set forth in SEQ ID NO:4, and a V_{H} region comprising an amino acid sequence as set forth in SEQ ID NO:9; (b) an anti-human LAG3 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In another specific embodiment, the method of increasing T cell proliferation comprises contacting the T cells with: (a) an anti-human PD-1 monoclonal antibody or antigen binding fragment thereof that comprises a V_{L} region comprising an amino acid sequence as set forth in SEQ ID NO:4, and a V_{H} region comprising an amino acid sequence as set forth in SEQ ID NO:9; (b) an anti-human LAG3 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In yet another specific embodiment, the method of enhancing T cell activity comprises contacting the T cells with: (a) an anti-human PD-1 monoclonal antibody or antigen binding fragment thereof that comprises a light chain comprising or consisting of an amino acid sequence as set forth in SEQ ID NO:5 and a heavy chain comprising or consisting of an amino acid sequence as set forth in SEQ ID NO:10; (b) an anti-human LAG3 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In yet another specific embodiment, the method of increasing cytokine production of T cells comprises contacting the T cells with: (a) an anti-human PD-1 monoclonal antibody or antigen binding fragment thereof that comprises a light chain comprising or consisting of an amino acid sequence as set forth in SEQ ID NO:5 and a heavy chain comprising or consisting of an amino acid sequence as set forth in SEQ ID NO:10; (b) an anti-human LAG3 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In yet another specific embodiment, the method of increasing T cell proliferation comprises contacting the T cells with: (a) an anti-human PD-1 monoclonal antibody or antigen binding fragment thereof that comprises a light chain comprising or consisting of an amino acid sequence as set forth in SEQ ID NO:5 and a heavy chain comprising or consisting of an amino acid sequence as set forth in SEQ ID NO:10; (b) an anti-human LAG3 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In one specific embodiment, the method of enhancing T cell activity comprises contacting the T cells with: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of increasing cytokine production of T cells comprises contacting the T cells with: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of increasing T cell proliferation comprises contacting the T cells with: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In another specific embodiment, the method of enhancing T cell activity comprises contacting the T cells with: (a) an anti-human PD-1 monoclonal antibody or antigen binding fragment thereof that comprises a V_{L} region comprising an amino acid sequence as set forth in SEQ ID NO:34, and a V_{H} region comprising an amino acid sequence as set forth in SEQ ID NO:39; (b) an anti-human LAG3 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In another specific embodiment, the method of increasing cytokine production of T cells comprises contacting the T cells with: (a) an anti-human PD-1 monoclonal antibody or antigen binding fragment thereof that comprises a V_{L} region comprising an amino acid sequence as set forth in SEQ ID NO:34, and a V_{H} region comprising an amino acid sequence as set forth in SEQ ID NO:39; (b) an anti-human LAG3 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In another specific embodiment, the method of increasing T cell proliferation comprises contacting the T cells with: (a) an anti-human PD-1 monoclonal antibody or antigen binding fragment thereof that comprises a V_{L} region comprising an amino acid sequence as set forth in SEQ ID NO:34, and a V_{H} region comprising an amino acid sequence as set forth in SEQ ID NO:39; (b) an anti-human LAG3 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In yet another specific embodiment, the method of enhancing T cell activity comprises contacting the T cells with: (a) an anti-human PD-1 monoclonal antibody or antigen binding fragment thereof that comprises a light chain comprising or consisting of an amino acid sequence as set forth in SEQ ID NO:35 and a heavy chain comprising or consisting of an amino acid sequence as set forth in SEQ ID NO:40; (b) an anti-human LAG3 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In yet another specific embodiment, the method of increasing cytokine production of T cells comprises contacting the T cells with: (a) an anti-human PD-1 monoclonal antibody or antigen binding fragment thereof that comprises a light chain comprising or consisting of an amino acid sequence as set forth in SEQ ID NO:35 and a heavy chain comprising or consisting of an amino acid sequence as set forth in SEQ ID NO:40; (b) an anti-human LAG3 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In yet another specific embodiment, the method of increasing T cell proliferation comprises contacting the T cells with: (a) an anti-human PD-1 monoclonal antibody or antigen binding fragment thereof that comprises a light chain comprising or consisting of an amino acid sequence as set forth in SEQ ID NO:35 and a heavy chain comprising or consisting of an amino acid sequence as set forth in SEQ ID NO:40; (b) an anti-human LAG3 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In some embodiments, the anti-human PD-1 monoclonal antibody is a human antibody. In other embodiments, the anti-human PD-1 monoclonal antibody is a humanized antibody.

In some embodiments, the anti-human LAG3 monoclonal antibody is a human antibody. In other embodiments, the anti-human LAG3 monoclonal antibody is a humanized antibody.

In some embodiments, the anti-human TIGIT monoclonal antibody is a human antibody. In other embodiments, the anti-human TIGIT monoclonal antibody is a humanized antibody.

Thus, in certain embodiments, provided herein is a method for enhancing T cell activity, comprising contacting the T cells with:
(a) a human or humanized anti-human PD-1 monoclonal antibody or antigen binding fragment thereof;
(b) a human or humanized anti-human LAG3 monoclonal antibody or antigen binding fragment thereof; and
(c) a human or humanized anti-human TIGIT monoclonal antibody or antigen binding fragment thereof.

In some embodiments, the enhancement of T cell activity occurs in vitro. In other embodiments, the enhancement of T cell activity occurs in vivo.

In some embodiments, provided herein is a method for increasing cytokine production of T cells, comprising contacting the T cells with:
(a) a human or humanized anti-human PD-1 monoclonal antibody or antigen binding fragment thereof;
(b) a human or humanized anti-human LAG3 monoclonal antibody or antigen binding fragment thereof; and
(c) a human or humanized anti-human TIGIT monoclonal antibody or antigen binding fragment thereof.

In some embodiments, the increased cytokine production of T cells occurs in vitro. In other embodiments, the increased cytokine production of T cells occurs in vivo.

In other embodiments, provided herein is a method for increasing proliferation of T cells, comprising contacting the T cells with:
(a) a human or humanized anti-human PD-1 monoclonal antibody or antigen binding fragment thereof;
(b) a human or humanized anti-human LAG3 monoclonal antibody or antigen binding fragment thereof; and
(c) a human or humanized anti-human TIGIT monoclonal antibody or antigen binding fragment thereof.

In some embodiments, the increased proliferation of T cells occurs in vitro. In other embodiments, the increased proliferation of T cells occurs in vivo.

Any combination of an anti-PD-1 monoclonal antibody or antigen binding fragment thereof disclosed in Section V.3, an anti-LAG3 monoclonal antibody or antigen binding fragment thereof disclosed in Section V.4, and an anti-TIGIT monoclonal antibody or antigen binding fragment thereof disclosed in Section V.5 are contemplated herein in methods of enhancing T cell activity (e.g., increasing cytokine production or increasing T cell proliferation).

In one specific embodiment, the anti-PD-1 monoclonal antibody or antigen binding fragment thereof is pembrolizumab. In another specific embodiment, the anti-PD-1 monoclonal antibody or antigen binding fragment thereof is nivolumab. In another specific embodiment, the anti-PD-1 monoclonal antibody or antigen binding fragment thereof is cemiplimab.

In certain embodiments of various methods of increasing cytokine production of T cells, the cytokine is selected from the group consisting of IL-1, IL-2, IL-6, IL-12, IL-17, IL-22, IL-23, GM-CSF, IFN-γ, and TNF-α. In one embodiment, the cytokine is IL-1. In another embodiment, the cytokine is IL-2. In yet another embodiment, the cytokine is IL-6. In still another embodiment, the cytokine is IL-12. In one embodiment, the cytokine is IL-17. In another embodiment, the cytokine is IL-22. In yet another embodiment, the cytokine is IL-23. In still another embodiment, the cytokine is GM-CSF. In one embodiment, the cytokine is IFN-γ. In another embodiment, the cytokine is TNF-α. In some embodiments, the cytokine is one, two, three, four, five, six, seven, eight, nine, or ten cytokines selected from the group consisting of IL-1, IL-2, IL-6, IL-12, IL-17, IL-22, IL-23, GM-CSF, IFN-γ, and TNF-α.

8. Dosing and Administration

Further provided herein are dosing regimens and routes of administration for treating cancer (e.g., colorectal cancer) or an infectious disease (e.g., a viral infection) using a combination of an anti-human PD-1 monoclonal antibody or antigen binding fragment thereof, an anti-human LAG3 monoclonal antibody or antigen binding fragment thereof, and an anti-human TIGIT monoclonal antibody or antigen binding fragment thereof.

The anti-PD-1 monoclonal antibody or antigen binding fragment thereof, the anti-LAG3 monoclonal antibody or antigen binding fragment thereof, and anti-TIGIT monoclonal antibody or antigen binding fragment thereof disclosed herein may be administered by continuous infusion, or by doses administered. e.g., daily, 1-7 times per week, weekly, bi-weekly, tri-weekly, every four weeks, every five weeks, every 6 weeks, monthly, bimonthly, quarterly, semiannually, annually, etc. Doses may be administered, e.g., intravenously, subcutaneously, topically, orally, nasally, rectally, intramuscular, intracerebrally, intraspinally, or by inhalation. In certain embodiments, the doses are administered intravenously. In certain embodiments, the doses are administered subcutaneously. A total dose for a treatment interval is generally at least 0.05 μg/kg body weight, more generally at least 0.2 μg/kg, 0.5 μg/kg, 1 μg/kg, 10 μg/kg, 100 μg/kg, 0.25 mg/kg, 1.0 mg/kg. 2.0 mg/kg, 5.0 mg/ml, 10 mg/kg, 25 mg/kg, 50 mg/kg or more. Doses may also be provided to achieve a pre-determined target concentration of the antibody (e.g., anti-PD-1 antibody) or antigen binding fragment thereof in the subject's serum, such as 0.1, 0.3, 1, 3, 10, 30, 100, 300 μg/mL or more.

In some embodiments, the anti-PD-1 monoclonal antibody or antigen binding fragment thereof is administered subcutaneously or intravenously, on a weekly, biweekly, triweekly, every 4 weeks, every 5 weeks, every 6 weeks, monthly, bimonthly, or quarterly basis at 10, 20, 50, 80, 100, 200, 300, 400, 500, 1000 or 2500 mg/subject. In some specific methods, the dose of the anti-PD-1 monoclonal antibody or antigen binding fragment thereof is from about 0.01 mg/kg to about 50 mg/kg, from about 0.05 mg/kg to about 25 mg/kg, from about 0.1 mg/kg to about 10 mg/kg, from about 0.2 mg/kg to about 9 mg/kg, from about 0.3 mg/kg to about 8 mg/kg, from about 0.4 mg/kg to about 7 mg/kg, from about 0.5 mg/kg to about 6 mg/kg, from about 0.6 mg/kg to about 5 mg/kg, from about 0.7 mg/kg to about 4 mg/kg, from about 0.8 mg/kg to about 3 mg/kg, from about 0.9 mg/kg to about 2 mg/kg, from about 1.0 mg/kg to about 1.5 mg/kg, from about 1.0 mg/kg to about 2.0 mg/kg, from about 1.0 mg/kg to about 3.0 mg/kg, from about 2.0 mg/kg to about 4.0 mg/kg. In some specific methods, the dose of the anti-PD-1 monoclonal antibody or antigen binding fragment thereof is from about 10 mg to about 500 mg, from about 25 mg to about 500 mg, from about 50 mg to about 500 mg, from about 100 mg to about 500 mg, from about 200 mg to about 500 mg, from about 150 mg to about 250 mg, from about 175 mg to about 250 mg, from about 200 mg to about 250 mg, from about 150 mg to about 240 mg, from about 175 mg to about 240 mg, from about 200 mg to about 240 mg. In some embodiments, the dose of the anti-PD-1 monoclonal antibody or antigen binding fragment thereof is 50 mg, 75 mg, 100 mg, 125 mg, 150 mg, 175 mg, 200 mg, 225 mg, 240 mg, 250 mg, 300 mg, 400 mg, or 500 mg.

In some embodiments, the anti-LAG3 monoclonal antibody or antigen binding fragment thereof is administered subcutaneously or intravenously, on a weekly, biweekly, triweekly, every 4 weeks, every 5 weeks, every 6 weeks, monthly, bimonthly, or quarterly basis at 10, 20, 50, 80, 100, 200, 500, 1000 or 2500 mg/subject. In some specific methods, the dose of the anti-LAG3 monoclonal antibody or antigen binding fragment thereof is from about 0.01 mg/kg to about 50 mg/kg, from about 0.05 mg/kg to about 25 mg/kg, from about 0.1 mg/kg to about 10 mg/kg, from about 0.2 mg/kg to about 9 mg/kg, from about 0.3 mg/kg to about 8 mg/kg, from about 0.4 mg/kg to about 7 mg/kg, from about 0.5 mg/kg to about 6 mg/kg, from about 0.6 mg/kg to about 5 mg/kg, from about 0.7 mg/kg to about 4 mg/kg, from about 0.8 mg/kg to about 3 mg/kg, from about 0.9 mg/kg to about 2 mg/kg, from about 1.0 mg/kg to about 1.5 mg/kg, from about 1.0 mg/kg to about 2.0 mg/kg, from about 1.0 mg/kg to about 3.0 mg/kg, from about 2.0 mg/kg to about 4.0 mg/kg. In some specific methods, the dose of the anti-LAG3 monoclonal antibody or antigen binding fragment thereof is from about 10 mg to about 500 mg, from about 25 mg to about 500 mg, from about 50 mg to about 500 mg, from about 100 mg to about 500 mg, from about 200 mg to about 500 mg, from about 150 mg to about 250 mg, from about 175 mg to about 250 mg, from about 200 mg to about 250 mg, from about 150 mg to about 240 mg, from about 175 mg to about 240 mg, from about 200 mg to about 240 mg. In some embodiments, the dose of the anti-LAG3 monoclonal antibody or antigen binding fragment thereof is 50 mg, 75 mg, 100 mg, 125 mg, 150 mg, 175 mg, 200 mg, 225 mg, 240 mg, 250 mg, 300 mg, 400 mg, or 500 mg.

In some embodiments, the anti-TIGIT monoclonal antibody or antigen binding fragment thereof is administered subcutaneously or intravenously, on a weekly, biweekly, triweekly, every 4 weeks, every 5 weeks, every 6 weeks, monthly, bimonthly, or quarterly basis at 10, 20, 50, 80, 100, 200, 500, 1000 or 2500 mg/subject. In some specific methods, the dose of the anti-TIGIT monoclonal antibody or antigen binding fragment thereof is from about 0.01 mg/kg to about 50 mg/kg, from about 0.05 mg/kg to about 25 mg/kg, from about 0.1 mg/kg to about 10 mg/kg, from about 0.2 mg/kg to about 9 mg/kg, from about 0.3 mg/kg to about 8 mg/kg, from about 0.4 mg/kg to about 7 mg/kg, from about 0.5 mg/kg to about 6 mg/kg, from about 0.6 mg/kg to about 5 mg/kg, from about 0.7 mg/kg to about 4 mg/kg, from about 0.8 mg/kg to about 3 mg/kg, from about 0.9 mg/kg to about 2 mg/kg, from about 1.0 mg/kg to about 1.5 mg/kg, from about 1.0 mg/kg to about 2.0 mg/kg, from about 1.0 mg/kg to about 3.0 mg/kg, from about 2.0 mg/kg to about 4.0 mg/kg. In some specific methods, the dose of the anti-TIGIT monoclonal antibody or antigen binding fragment thereof is from about 10 mg to about 500 mg, from about 25 mg to about 500 mg, from about 50 mg to about 500 mg, from about 100 mg to about 500 mg, from about 200 mg to about 500 mg, from about 150 mg to about 250 mg, from about 175 mg to about 250 mg, from about 200 mg to about 250 mg, from about 150 mg to about 240 mg, from about 175 mg to about 240 mg, from about 200 mg to about 240 mg. In some embodiments, the dose of the anti-TIGIT monoclonal antibody or antigen binding fragment thereof is 50 mg, 75 mg, 100 mg, 125 mg, 150 mg, 175 mg, 200 mg, 225 mg, 240 mg, 250 mg, 300 mg, 400 mg, or 500 mg.

In a specific embodiment, the anti-human PD-1 monoclonal antibody or antigen binding fragment thereof is pembrolizumab. In another specific embodiment, the anti-human PD-1 monoclonal antibody or antigen binding fragment thereof is nivolumab. In another specific embodiment, the anti-human PD-1 monoclonal antibody or antigen binding fragment thereof is cemiplimab.

Thus, in certain embodiments, the human patient is administered:
(a) 200 mg of pembrolizumab;
(b) an anti-human LAG3 monoclonal antibody or antigen binding fragment thereof; and
(c) an anti-human TIGIT monoclonal antibody or antigen binding fragment thereof;
wherein pembrolizumab is administered once every three weeks.

In some embodiments, the human patient is administered:
(a) 240 mg of pembrolizumab;
(b) an anti-human LAG3 monoclonal antibody or antigen binding fragment thereof; and
(c) an anti-human TIGIT monoclonal antibody or antigen binding fragment thereof;
wherein pembrolizumab is administered once every three weeks.

In other embodiments, the human patient is administered:
(a) 2 mg/kg of pembrolizumab;
(b) an anti-human LAG3 monoclonal antibody or antigen binding fragment thereof; and
(c) an anti-human TIGIT monoclonal antibody or antigen binding fragment thereof;
wherein pembrolizumab is administered once every three weeks.

In some embodiments, the human patient is administered:
(a) 400 mg of pembrolizumab;
(b) an anti-human LAG3 monoclonal antibody or antigen binding fragment thereof; and
(c) an anti-human TIGIT monoclonal antibody or antigen binding fragment thereof;
wherein pembrolizumab is administered once every six weeks.

In some embodiments, the human patient is administered:
(a) 240 mg of nivolumab;
(b) an anti-human LAG3 monoclonal antibody or antigen binding fragment thereof; and
(c) an anti-human TIGIT monoclonal antibody or antigen binding fragment thereof;
wherein nivolumab is administered once every two weeks.

In other embodiments, the human patient is administered:
(a) 3 mg/kg of nivolumab;
(b) an anti-human LAG3 monoclonal antibody or antigen binding fragment thereof; and
(c) an anti-human TIGIT monoclonal antibody or antigen binding fragment thereof;
wherein nivolumab is administered once every two weeks.

In some embodiments, the human patient is administered:
(a) 350 mg of cemiplimab;
(b) an anti-human LAG3 monoclonal antibody or antigen binding fragment thereof; and
(c) an anti-human TIGIT monoclonal antibody or antigen binding fragment thereof;
wherein pembrolizumab is administered once every three weeks.

In certain embodiments, the human patient is administered:
(a) 200 mg of an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively;
(b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively;
wherein the anti-human PD-1 monoclonal antibody or antigen binding fragment thereof is administered once every three weeks.

In some embodiments, the human patient is administered:
(a) 240 mg of an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively;
(b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively;
wherein the anti-human PD-1 monoclonal antibody or antigen binding fragment thereof is administered once every three weeks.

In other embodiments, the human patient is administered:
(a) 2 mg/kg of an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively;
(b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively;
wherein the anti-human PD-1 monoclonal antibody or antigen binding fragment thereof is administered once every three weeks.

In some embodiments, the human patient is administered:
(a) 400 mg of an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively;
(b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively;
wherein the anti-human PD-1 monoclonal antibody or antigen binding fragment thereof is administered once every six weeks.

In some embodiments, the human patient is administered:
(a) 240 mg of an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively;
(b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively;
wherein the anti-human PD-1 monoclonal antibody or antigen binding fragment thereof is administered once every two weeks.

In other embodiments, the human patient is administered:
(a) 3 mg/kg of an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively;
(b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively;
wherein the anti-human PD-1 monoclonal antibody or antigen binding fragment thereof is administered once every two weeks.

In certain embodiments, the human patient is administered:
(a) 200 mg of pembrolizumab;
(b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively;
wherein pembrolizumab is administered once every three weeks.

In some embodiments, the human patient is administered:
(a) 240 mg of pembrolizumab;
(b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively;
wherein pembrolizumab is administered once every three weeks.

In other embodiments, the human patient is administered:
(a) 2 mg/kg of pembrolizumab;
(b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively;
wherein pembrolizumab is administered once every three weeks.

In some embodiments, the human patient is administered:
(a) 400 mg of pembrolizumab;
(b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively;
wherein pembrolizumab is administered once every six weeks.

In some embodiments, the human patient is administered:
(a) 240 mg of nivolumab;
(b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively;
wherein nivolumab is administered once every two weeks.

In other embodiments, the human patient is administered:
(a) 3 mg/kg of nivolumab;
(b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively;
wherein nivolumab is administered once every two weeks.

In some embodiments, the human patient is administered:
(a) 350 mg of cemiplimab;
(b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively;
wherein pembrolizumab is administered once every three weeks.

In some embodiments, at least one of the therapeutic agents (e.g., the anti-PD-1 monoclonal antibody or binding fragment thereof, the anti-LAG3 monoclonal antibody or binding fragment thereof, or the anti-TIGIT monoclonal antibody or binding fragment thereof) in the combination therapy is administered using the same dosage regimen (dose, frequency, and duration of treatment) that is typically employed when the agent is used as monotherapy for treating the same condition. In other embodiments, the patient receives a lower total amount of at least one of the therapeutic agents (e.g., the anti-PD-1 monoclonal antibody or binding fragment thereof, the anti-LAG3 monoclonal antibody or binding fragment thereof, or the anti-TIGIT monoclonal antibody or binding fragment thereof) in the combination therapy than when the agent is used as monotherapy, e.g., smaller doses, less frequent doses, and/or shorter treatment duration.

A combination therapy disclosed herein may be used prior to or following surgery to remove a tumor and may be used prior to, during, or after radiation treatment.

In some embodiments, a combination therapy disclosed herein is administered to a patient who has not previously been treated with a biotherapeutic or chemotherapeutic agent, i.e., is treatment-naïve. In other embodiments, the combination therapy is administered to a patient who failed to achieve a sustained response after prior therapy with the biotherapeutic or chemotherapeutic agent, i.e., is treatment-experienced.

The therapeutic combination disclosed herein may be used in combination with one or more other active agents, including but not limited to, other anti-cancer agents that are used in the prevention, treatment, control, amelioration, or reduction of risk of a particular disease or condition (e.g., cancer). Such other active agents may be administered, by a route and in an amount commonly used therefor, contemporaneously or sequentially with a therapeutic combination of the present disclosure.

The one or more additional active agents may be co-administered with the anti-PD-1 monoclonal antibody or antigen binding fragment thereof, the anti-LAG3 monoclonal antibody or antigen binding fragment thereof, or the anti-TIGIT monoclonal antibody or antigen binding fragment thereof. The additional active agent(s) can be administered in a single dosage form with one or more co-administered agent selected from the anti-PD-1 monoclonal antibody or antigen binding fragment thereof, the anti-LAG3 monoclonal antibody or antigen binding fragment thereof, and the anti-TIGIT monoclonal antibody or antigen binding fragment thereof. The additional active agent(s) can also be administered in separate dosage form(s) from the dosage forms containing the anti-PD-1 monoclonal antibody or antigen binding fragment thereof, the anti-LAG3 monoclonal antibody or antigen binding fragment thereof, and/or the anti-TIGIT monoclonal antibody or antigen binding fragment thereof.

The additional active agent can be, e.g., a chemotherapeutic, a biotherapeutic agent (including but not limited to antibodies or antigen binding fragments thereof that specifically bind to an antigen selected from the group consisting of: PD-L1, PD-L2, CTLA4, BTLA, TIM3, HVEM, GITR, CD27, ILT2, ILT3, ILT4, ILT5, SIRPα, NKG2A, NKG2C, NKG2E, TSLP, IL10, VISTA, VEGF, EGFR, Her2/neu, VEGF receptors, other growth factor receptors, CD20, CD28, CD40, CD-40L, CD70, OX-40, 4-1BB, and ICOS).

The additional active agent can be selected from the group consisting of STING agonists, poly ADP ribose polymerase (PARP) inhibitors, mitogen-activated protein kinase (MEK) inhibitors, cyclin-dependent kinase (CDK) inhibitors, indoleamine 2,3-dioxygenase (IDO) inhibitors, tryptophan 2,3-dioxygenase (TDO) selective inhibitors, anti-viral compounds, antigens, adjuvants, anti-cancer agents, CTLA-4 pathway antagonists, lipids, liposomes, peptides, cytotoxic agents, chemotherapeutic agents, immunomodulatory cell lines, checkpoint inhibitors, vascular endothelial growth factor (VEGF) receptor inhibitors, topoisomerase II inhibitors, smoothen inhibitors, alkylating agents, anti-tumor antibiotics, anti-metabolites, retinoids, and immunomodulatory agents including but not limited to anti-cancer vaccines.

The additional active agent can be an anti-viral compound, including but not limited to, hepatitis B virus (HBV) inhibitors, hepatitis C virus (HCV) protease inhibitors, HCV polymerase inhibitors, HCV NS4A inhibitors, HCV NS5A inhibitors, HCV NS5b inhibitors, and human immunodeficiency virus (HIV) inhibitors.

The additional active agent can be an cytotoxic agent, including but not limited to, arsenic trioxide (sold under the tradename TRISENOX®), asparaginase (also known as L-asparaginase, and Erwinia L-asparaginase, sold under the tradenames ELSPAR® and KIDROLASE®).

The additional active agent can be an chemotherapeutic agent, including but not limited to, abiraterone acetate, altretamine, anhydrovinblastine, auristatin, bexarotene, bicalutamide, BMS 184476, 2,3,4,5,6-pentafluoro-N-(3-fluoro-4-methoxyphenyl)benzene sulfonamide, bleomycin, N,N-dimethyl-L-valyl-L-valyl-N-methyl-L-valyl-L-prolyl-1-Lproline-t-butylamide, cachectin, cemadotin, chlorambucil, cyclophosphamide, 3′,4′-didehydro-4′deoxy-8′-norvin-caleukoblastine, dinaciclib, docetaxol, doxetaxel, cyclophosphamide, carboplatin, carmustine, cisplatin, cryptophycin, cyclophosphamide, cytarabine, dacarbazine (DTIC), dactinomycin, daunorubicin, decitabine dolastatin, doxorubicin (adriamycin), etoposide, 5-fluorouracil, finasteride, flutamide, hydroxyurea and hydroxyurea andtaxanes, ifosfamide, liarozole, lonidamine, lomustine, MDV3100, mechlorethamine (nitrogen mustard), melphalan, mivobulin isethionate, rhizoxin, sertenef, streptozocin, mitomycin, methotrexate, taxanes, nilutamide, olaparib, onapristone, paclitaxel, prednimustine, procarbazine, RPR109881, selumetinib, stramustine phosphate, tamoxifen, tasonermin, taxol, tretinoin, vinblastine, vincristine, vindesine sulfate, and vinflunine, and pharmaceutically acceptable salts thereof.

The additional active agent can be a vascular endothelial growth factor (VEGF) receptor inhibitors, including but not limited to, bevacizumab (sold under the trademark AVASTIN by Genentech/Roche), axitinib (described in PCT International Patent Publication No. WO01/002369), Brivanib Alaninate ((S)-((R)-1-(4-(4-Fluoro-2-methyl-1H-indol-5-yloxy)-5-methylpyrrolo[2,1-f][1,2,4]triazin-6-yloxy)propan-2-yl)2-aminopropanoate, also known as BMS-582664), motesanib (N-(2,3-dihydro-3,3-dimethyl-1H-indol-6-yl)-2-[(4-pyridinylmethyl)amino]-3-pyridinecarboxamide, and described in PCT International Patent Application Publication No. WO02/068470), pasireotide (also known as SO 230, and described in PCT International Patent Publication No. WO02/010192), and sorafenib. The additional active agent can be a topoisomerase II inhibitor, including but not limited to, etoposide and teniposide.

The additional active agent can be an alkylating agent, including but not limited to, 5-azacytidine, decitabine, temozolomide, dactinomycin (also known as actinomycin-D, melphalan, altretamine, carmustine, bendamustine, busulfan, carboplatin, lomustine, cisplatin, chlorambucil, cyclophosphamide, dacarbazine, altretamine, ifosfamide, procarbazine, mechlorethamine, streptozocin, thiotepa, and pharmaceutically acceptable salts thereof.

The additional active agent can be an anti-tumor antibiotic, including but not limited to, doxorubicin, bleomycin, daunorubicin daunorubicin liposomal (daunorubicin citrate liposome), mitoxantrone, epirubicin, idarubicin, and mitomycin C.

The additional active agent can be an anti-metabolite, including but not limited to, claribine, 5-fluorouracil, 6-thioguanine, pemetrexed (sold under the tradename ALIMTA®), cytarabine (also known as arabinosylcytosine (Ara-C)), cytarabine liposomal (also known as Liposomal Ara-C, sold under the tradename DEPOCYTTM™), decitabine (sold under the tradename DACOGEN®), hydroxyurea and fludarabine, floxuridine, cladribine (also known as 2-chlorodeoxyadenosine (2-CdA), methotrexate (also known as amethopterin, methotrexate sodium (MTX)), and pentostatin.

The additional active agent can be a retinoid, including but not limited to, alitretinoin, tretinoin, isotretinoin, and bexarotene.

9. Pharmaceutical Compositions

In yet another aspect, provided herein are pharmaceutical compositions comprising:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In certain embodiments, the pharmaceutical composition further comprises a pharmaceutically acceptable carrier.

The pharmaceutical compositions comprising an anti-human PD-1 monoclonal antibody or antigen binding fragment thereof, an anti-human LAG3 monoclonal antibody or antigen binding fragment thereof, and an anti-human TIGIT monoclonal antibody or antigen binding fragment thereof can be prepared for storage by mixing the antibodies having the desired degree of purity with optionally physiologically acceptable carriers, excipients, or stabilizers (see. e.g., Remington, Remington's Pharmaceutical Sciences (18^{th} ed. 1980)) in the form of aqueous solutions or lyophilized or other dried forms.

The pharmaceutically acceptable carriers, excipients, or stabilizers are non-toxic to the cell or mammalian being exposed thereto at the dosage and concentrations employed. Often the pharmaceutically acceptable carrier is an aqueous pH buffered solution. Examples of pharmaceutically acceptable carriers include buffers, such as phosphate, citrate, acetate, and other organic acids; antioxidants, such as ascorbic acid; low molecular weight (e.g., fewer than about 10 amino acid residues) polypeptide; proteins, such as serum albumin, gelatin, or immunoglobulin; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. The pharmaceutically acceptable carriers can also refer to a diluent, adjuvate (e.g., Freund's adjuvate (complete or incomplete)), excipient, or vehicle. Such carriers can be sterile liquids, such as water and oils, including those of petroleum, animal, vegetable, or synthetic origin, such as peanut oil, soybean oil, mineral oil, sesame oil, and the like. Water is an exemplary carrier when a composition (e.g., a pharmaceutical composition) is administered intravenously. Saline solutions and aqueous dextrose and glycerol solutions can also be employed as liquid carriers, particularly for injectable solutions. Suitable excipients (e.g., pharmaceutical excipients) include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsions, tablets, pills, capsules, powders, sustained-release formulations, and the like.

In one embodiment, provided herein is a pharmaceutical composition comprising:
(a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively;
(b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In another embodiment, provided herein is a pharmaceutical composition comprising:
(a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively;
(b) an anti-human LAG3 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In yet another embodiment, provided herein is a pharmaceutical composition comprising:
(a) an anti-human PD-1 monoclonal antibody or antigen binding fragment thereof that comprises a V_{L} region comprising an amino acid sequence as set forth in SEQ ID NO:4, and a V_{H} region comprising an amino acid sequence as set forth in SEQ ID NO:9;
(b) an anti-human LAG3 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and
(c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In still another embodiment, provided herein is a pharmaceutical composition comprising:
(a) an anti-human PD-1 monoclonal antibody or antigen binding fragment thereof that comprises a V_{L} region comprising an amino acid sequence as set forth in SEQ ID NO:34, and a V_{H} region comprising an amino acid sequence as set forth in SEQ ID NO:39;
(b) an anti-human LAG3 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and
(c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In yet another embodiment, provided herein is a pharmaceutical composition comprising:
(a) an anti-human PD-1 monoclonal antibody or antigen binding fragment thereof that comprises a light chain comprising or consisting of an amino acid sequence as set forth in SEQ ID NO:5 and a heavy chain comprising or consisting of an amino acid sequence as set forth in SEQ ID NO:10;
(b) an anti-human LAG3 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and
(c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In still another embodiment, provided herein is a pharmaceutical composition comprising:
(a) an anti-human PD-1 monoclonal antibody or antigen binding fragment thereof that comprises a light chain comprising or consisting of an amino acid sequence as set forth in SEQ ID NO:35 and a heavy chain comprising or consisting of an amino acid sequence as set forth in SEQ ID NO:40;
(b) an anti-human LAG3 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and
(c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In some embodiments, the anti-PD-1 monoclonal antibody or antigen binding fragment thereof is pembrolizumab. In some embodiments, the anti-PD-1 monoclonal antibody or antigen binding fragment thereof is nivolumab. In some embodiments, the anti-PD-1 monoclonal antibody or antigen binding fragment thereof is cemiplimab.

In various embodiments, the pharmaceutical compositions disclosed herein can further comprise one or more active ingredients (such as an anti-cancer agent) described in Section V.8.

10. Kits

In still another aspect, provided herein are kits comprising a therapeutic combination of the antibodies or antigen binding fragments thereof disclosed herein (e.g., an anti-PD-1 monoclonal antibody or antigen binding fragment thereof, an anti-LAG3 monoclonal antibody or antigen binding fragment thereof, and an anti-TIGIT monoclonal antibody or antigen binding fragment thereof) or a pharmaceutical composition thereof, packaged into suitable packaging material. A kit optionally includes a label or packaging insert that include a description of the components or instructions for use in vitro, in vivo, or ex vivo, of the components therein.

In some embodiments, the kit comprises
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In certain embodiments, the kit further comprises instructions for administering to a human patient the anti-human PD-1 monoclonal antibody or antigen binding fragment thereof, the anti-human LAG3 monoclonal antibody or antigen binding fragment thereof, and the anti-human TIGIT monoclonal antibody or antigen binding fragment thereof.

In one embodiment, the kit comprises: (a) a dosage of an anti-PD-1 monoclonal antibody or antigen binding fragment thereof; (b) a dosage of an anti-LAG3 monoclonal antibody or antigen binding fragment thereof; (c) a dosage of an anti-TIGIT monoclonal antibody or antigen binding fragment thereof; and (d) instructions for administering to a human patient the anti-human PD-1 monoclonal antibody or antigen binding fragment thereof, the anti-human LAG3 monoclonal antibody or antigen binding fragment thereof, and the anti-human TIGIT monoclonal antibody or antigen binding fragment thereof.

In some embodiments, the anti-PD-1 monoclonal antibody or antigen binding fragment thereof is pembrolizumab. In some embodiments, the anti-PD-1 monoclonal antibody or antigen binding fragment thereof is nivolumab. In some embodiments, the anti-PD-1 monoclonal antibody or antigen binding fragment thereof is cemiplimab.

In another embodiment, the kit comprises: (a) a dosage of 240 mg of pembrolizumab; (b) a dosage of an anti-LAG3 monoclonal antibody or antigen binding fragment thereof; (c) a dosage of an anti-TIGIT monoclonal antibody or antigen binding fragment thereof; and (d) instructions for administering to a human patient the anti-human PD-1 monoclonal antibody or antigen binding fragment thereof, the anti-human LAG3 monoclonal antibody or antigen binding fragment thereof, and the anti-human TIGIT monoclonal antibody or antigen binding fragment thereof.

In yet another embodiment, the kit comprises: (a) a dosage of 200 mg of pembrolizumab; (b) a dosage of an anti-LAG3 monoclonal antibody or antigen binding fragment thereof; (c) a dosage of an anti-TIGIT monoclonal antibody or antigen binding fragment thereof; and (d) instructions for administering to a human patient the anti-human PD-1 monoclonal antibody or antigen binding fragment thereof, the anti-human LAG3 monoclonal antibody or antigen binding fragment thereof, and the anti-human TIGIT monoclonal antibody or antigen binding fragment thereof.

In yet another embodiment, the kit comprises: (a) a dosage of 400 mg of pembrolizumab; (b) a dosage of an anti-LAG3 monoclonal antibody or antigen binding fragment thereof; (c) a dosage of an anti-TIGIT monoclonal antibody or antigen binding fragment thereof; and (d) instructions for administering to a human patient the anti-human PD-1 monoclonal antibody or antigen binding fragment thereof, the anti-human LAG3 monoclonal antibody or antigen binding fragment thereof, and the anti-human TIGIT monoclonal antibody or antigen binding fragment thereof.

In still another embodiment, the kit comprises: (a) a dosage of 240 mg of nivolumab; (b) a dosage of an anti-LAG3 monoclonal antibody or antigen binding fragment thereof; (c) a dosage of an anti-TIGIT monoclonal antibody or antigen binding fragment thereof; and (d) instructions for administering to a human patient the anti-human PD-1 monoclonal antibody or antigen binding fragment thereof, the anti-human LAG3 monoclonal antibody or antigen binding fragment thereof, and the anti-human TIGIT monoclonal antibody or antigen binding fragment thereof.

In yet another embodiment, the kit comprises: (a) a dosage of 350 mg of cemiplimab; (b) a dosage of an anti-LAG3 monoclonal antibody or antigen binding fragment thereof; (c) a dosage of an anti-TIGIT monoclonal antibody or antigen binding fragment thereof; and (d) instructions for administering to a human patient the anti-human PD-1 monoclonal antibody or antigen binding fragment thereof, the anti-human LAG3 monoclonal antibody or antigen binding fragment thereof, and the anti-human TIGIT monoclonal antibody or antigen binding fragment thereof.

In some embodiments, the kit comprises means for separately retaining the components, such as a container, divided bottle, or divided foil packet. A kit of this disclosure can be used for administration of different dosage forms, for example, oral and parenteral, for administration of the separate compositions at different dosage intervals, or for titration of the separate compositions against one another.

11. Uses of a Therapeutic Combination for Treating Cancer or Infection

In still another aspect, provided herein are uses of a therapeutic combination for treating cancer (e.g., colorectal cancer) or an infectious disease (e.g., a viral infection) in a human patient, wherein the therapeutic combination comprises:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In some embodiments, the cancer is selected from the group consisting of osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer (e.g., non-small cell lung cancer), gastric cancer, colorectal cancer, cervical cancer, synovial sarcoma, head and neck cancer, squamous cell carcinoma, lymphoma (e.g., DLBCL or NHL), multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, and carcinoid cancer.

In certain embodiments, the cancer is metastatic. In some embodiments, the cancer is relapsed. In other embodiments, the cancer is refractory. In yet other embodiments, the cancer is relapsed and refractory.

In one embodiment, the cancer is osteosarcoma. In another embodiment, the cancer is rhabdomyosarcoma. In yet another embodiment, the cancer is neuroblastoma. In still another embodiment, the cancer is kidney cancer. In one embodiment, the cancer is leukemia. In another embodiment, the cancer is renal transitional cell cancer. In yet another embodiment, the cancer is bladder cancer. In still another embodiment, the cancer is Wilm's cancer. In one embodiment, the cancer is ovarian cancer. In another embodiment, the cancer is pancreatic cancer. In yet another embodiment, the cancer is breast cancer. In still another embodiment, the cancer is prostate cancer. In one embodiment, the cancer is bone cancer. In another embodiment, the cancer is lung cancer. In yet another embodiment, the cancer is non-small cell lung cancer. In still another embodiment, the cancer is gastric cancer. In one embodiment, the cancer is colorectal cancer. In another embodiment, the cancer is cervical cancer. In yet another embodiment, the cancer is synovial sarcoma. In still another embodiment, the cancer is head and neck cancer. In one embodiment, the cancer is squamous cell carcinoma. In another embodiment, the cancer is lymphoma. In one embodiment, the cancer is DLBCL. In another embodiment, the cancer is NHL. In yet another embodiment, the cancer is multiple myeloma. In still another embodiment, the cancer is renal cell cancer. In one embodiment, the cancer is retinoblastoma. In another embodiment, the cancer is hepatoblastoma. In yet another embodiment, the cancer is hepatocellular carcinoma. In still another embodiment, the cancer is melanoma. In one embodiment, the cancer is rhabdoid tumor of the kidney. In another embodiment, the cancer is Ewing's sarcoma. In yet another embodiment, the cancer is chondrosarcoma. In still another embodiment, the cancer is brain cancer. In one embodiment, the cancer is glioblastoma. In another embodiment, the cancer is meningioma. In yet another embodiment, the cancer is pituitary adenoma. In still another embodiment, the cancer is vestibular schwannoma. In one embodiment, the cancer is primitive neuroectodermal tumor. In another embodiment, the cancer is medulloblastoma. In yet another embodiment, the cancer is astrocytoma. In still another embodiment, the cancer is anaplastic astrocytoma. In one embodiment, the cancer is oligodendroglioma. In another embodiment, the cancer is ependymoma. In yet another embodiment, the cancer is choroid plexus papilloma. In still another embodiment, the cancer is polycythemia vera. In one embodiment, the cancer is thrombocythemia. In another embodiment, the cancer is idiopathic myelfibrosis. In yet another embodiment, the cancer is soft tissue sarcoma. In still another embodiment, the cancer is thyroid cancer. In one embodiment, the cancer is endometrial cancer. In another embodiment, the cancer is carcinoid cancer. In yet another embodiment, the cancer is refractory head and neck cancer. In still another embodiment, the cancer is relapsed/refractory NHL (rrNHL). In yet still another embodiment, the cancer is PD-1 refractory rrNHL.

In one embodiment, provided herein is use of a therapeutic combination for treating colorectal cancer in a human patient, wherein the therapeutic combination comprises:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In some embodiments, provided herein is use of a therapeutic combination for treating gastric cancer in a human patient, wherein the therapeutic combination comprises:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In other embodiments, provided herein is use of a therapeutic combination for treating head and neck cancer in a human patient, wherein the therapeutic combination comprises:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In yet other embodiments, provided herein is use of a therapeutic combination for treating refractory head and neck cancer in a human patient, wherein the therapeutic combination comprises:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In still other embodiments, provided herein is use of a therapeutic combination for treating lung cancer in a human patient, wherein the therapeutic combination comprises:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In certain embodiments, provided herein is use of a therapeutic combination for treating non-small cell lung cancer in a human patient, wherein the therapeutic combination comprises:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In some embodiments, provided herein is use of a therapeutic combination for treating breast cancer in a human patient, wherein the therapeutic combination comprises:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In other embodiments, provided herein is use of a therapeutic combination for treating cervical cancer in a human patient, wherein the therapeutic combination comprises:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In yet other embodiments, provided herein is use of a therapeutic combination for treating ovarian cancer in a human patient, wherein the therapeutic combination comprises:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In still other embodiments, provided herein is use of a therapeutic combination for treating DLBCL in a human patient, wherein the therapeutic combination comprises:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In certain embodiments, provided herein is use of a therapeutic combination for treating NHL in a human patient, wherein the therapeutic combination comprises:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In some embodiments, provided herein is use of a therapeutic combination for treating rrNHL in a human patient, wherein the therapeutic combination comprises:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In other embodiments, provided herein is use of a therapeutic combination for treating naïve rrNHL in a human patient, wherein the therapeutic combination comprises:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In yet other embodiments, provided herein is use of a therapeutic combination for treating PD-1 refractory rrNHL in a human patient, wherein the therapeutic combination comprises:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In still other embodiments, provided herein is use of a therapeutic combination for treating an infectious disease, wherein the therapeutic combination comprises:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof.

In one embodiment, the infectious disease is viral infection. In another embodiment, the infectious disease is bacterial infection. In yet another embodiment, the infectious disease is parasitic infection. In still another embodiment, the infectious disease is fungal infection.

In certain embodiments, the viral infection is infection with a virus selected from the group consisting of human immunodeficiency virus (HIV), ebola virus, hepatitis virus A (HAV), hepatitis virus B (HBV), hepatitis virus C (HCV), herpes virus (e.g., VZV, HSV-I, HAV-6, HSV-II, CMV, Epstein Barr virus), adenovirus, influenza virus, flavivirus, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus, and arboviral encephalitis virus.

In some embodiments, the bacterial infection is infection with a bacteria selected from the group consisting of chlamydia, rickettsia, mycobacteria, staphylococci, streptococci, pneumonococci, meningococci, gonococci, klebsiella, proteus, serratia, pseudomonas, legionella, salmonella, bacilli, borriella, Corynebacterium diphtheriae, Vibrio cholerae, Clostridium tetan, Clostridium botulinum, Bacillus anthricis, Yersinia pestis, Mycobacterium leprae, and Mycobacterium lepromatosis.

In other embodiments, the fungal infection is infection with a fungus selected from the group consisting of Candida (albicans, krusei, glabrata, tropicalis, etc.), Cryptococcus neoformans, Aspergillus (fumigatus, niger, etc.), Genus Mucorales (mucor, absidia, rhizopus), Sporothrix schenkii, Blastomyces dermatitidis, Paracoccidioides brasiliensis, Coccidioides immitis and Histoplasma capsulatum.

In yet other embodiments, the parasitic infection is infection with a parasite selected from the group consisting of Entamoeba histolytica, Balantidium coli, Naegleria fowleri, Acanthamoeba, Giardia lambia, Cryptosporidium, Pneumocystis carinii, Plasmodium vivax, Babesia microti, Trypanosoma brucei, Trypanosoma cruzi, Leishmania donovani, Toxoplasma gondii, Nippostrongylus brasiliensis.

In some embodiments, the anti-PD-1 monoclonal antibody or antigen binding fragment thereof is an anti-human PD-1 monoclonal antibody or antigen binding fragment thereof as disclosed in Section V.3.

In some embodiments, the anti-LAG3 monoclonal antibody or antigen binding fragment thereof is an anti-human LAG3 monoclonal antibody or antigen binding fragment thereof as disclosed in Section V.4.

In some embodiments, the anti-TIGIT monoclonal antibody or antigen binding fragment thereof is an anti-human TIGIT monoclonal antibody or antigen binding fragment thereof as disclosed in Section V.5.

In some embodiments, the anti-PD-1 monoclonal antibody or antigen binding fragment thereof is pembrolizumab. In some embodiments, the anti-PD-1 monoclonal antibody or antigen binding fragment thereof is nivolumab. In some embodiments, the anti-PD-1 monoclonal antibody or antigen binding fragment thereof is cemiplimab.

A number of embodiments of the invention have been described. It will be understood that various modifications may be made without departing from the spirit and scope of the invention. It will be further understood that each embodiment may be combined with one or more other embodiments, to the extent that such a combination is consistent with the description of the embodiments. Accordingly, the following examples are intended to illustrate but not limit the scope of the invention described in the claims.

### VI. EXAMPLES

The examples in this section (section VI) are offered by way of illustration, and not by way of limitation.

### 6.1 Example 1: A Combination of an Anti-mouse PD-1 Antibody, an Anti-mouse LAG3 Antibody, and an Anti-mouse TIGIT Antibody Exhibited Anti-Tumor Effect in Syngeneic Mouse Carcinoma Models

Three syngeneic mouse carcinoma models were used for studying the anti-tumor efficacy of a combination of three anti-mouse monoclonal antibodies (anti-mouse PD-1, anti-mouse LAG3, and anti-mouse TIGIT mAbs). These models include CT-26 colon carcinoma model, MBT-2 bladder carcinoma model, and RENCA renal carcinoma model.

6.1.1 Antibodies

Surrogate anti-mouse PD-1, anti-mouse LAG3, and anti-mouse TIGIT monoclonal antibodies and isotype control antibodies are described in Table 3.

TABLE 3
Antibodies and Formulation of Antibodies

| Antibody | | | | |
|---|---|---|---|---|
| Name | Target | Isotype | Source | Formulation |
| mIgG1 | Adenoviral | Mouse IgG1 | Merck | 20 mM sodium |
| D265A | hexon 25 | D265A/kappa | Research | acetate, 9% |
| isotype | | | Laboratories | sucrose, pH 5.5 |
| control | | | | |
| mIgG1 | Adenoviral | Mouse IgG1 | Merck | 10 mM sodium |
| isotype | hexon 25 | kappa | Research | phosphate, 75 |
| control | | | Laboratories | mM sodium |
| | | | | chloride, 3% |
| | | | | sucrose, pH 7.3 |
| mIgG2a | Unknown | Mouse | BioXCell | Phosphate- |
| isotype | specificity | IgG2a/kappa | Cat. No. | buffered saline, |
| control | | | BE0085 | pH 7.0 |
| muDX400 | Mouse | Mouse IgG1 | Merck | 20 mM sodium |
| | PD-1 | D265A/kappa | Research | acetate, 9% |
| | | | Laboratories | sucrose, pH 5.5 |
| c28G10 | Mouse | Rat/mouse | Merck | 20 mM sodium |
| | LAGS | chimera, mouse | Research | acetate, 9% |
| | | IgG1 | Laboratories | sucrose, pH 5.5 |
| | | D265A/kappa | | |
| c18G10 | Mouse | Rat/mouse | Merck | 20 mM sodium |
| | TIGIT | chimera, mouse | Research | acetate, 9% |
| | | IgG1 | Laboratories | sucrose, pH 5.5 |
| | | D265A/kappa | | |

Stocks of the antibodies were stored frozen at −80° C., and dosing solutions were prepared by diluting the antibodies in their respective formulation solutions.

6.1.2 Animals

Details of animals used for each carcinoma model are shown in Table 4.

TABLE 4
Animals Used for Mouse Carcinoma Model Studies

| | | | Mean body |
|---|---|---|---|
| | | | weight at |
| | | | enrollment |
| Model | Animals | Vendor | (Day 0) |
| CT-26 | 7-8 week old female BALB/ | Taconic | 20 g |
| | cAnNTaC | Biosciences, | |
| MBT-2 | 8-12 week old female C3H/ | Inc. (Hudson, | 25 g |
| | HeNTac | NY) | |
| RENCA | 8-12 week old female BALB/ | | 19 g |
| | cAnNTaC | | |

Conventional animal chow and water were provided ad libitum. Animals were housed for one week prior to the start of the study.

6.1.3 Tumor Cell Lines

CT-26 (ATCC, Manassas, VA) was cultured in DMEM supplemented with 10% fetal bovine serum.

MBT-2 (MRL, Palo Alto, CA) was cultured in DMEM supplemented with 10% heat-inactivated fetal bovine serum.

RENCA (ATCC, Manassas, VA) was cultured in RPMI 1640 supplemented with 10% heat-inactivated fetal bovine serum.

6.1.4 Tumor Measurements and Body Weights

Tumor length and width were measured using electronic calipers, and tumor volume was determined using the formula Volume (mm³)=0.5×Length×Width², where length is the longer dimension. Animals were weighed and tumors were measured twice a week. To prevent bias, any outliers by weight or tumor volume were removed and the remaining mice were assigned into groups of 10 based on tumor volumes. If tumor volume reached or exceeded 2000 mm³ before the scheduled take down, animals were euthanized.

6.1.5 Tumor Challenge Experiments

6.1.5.1 CT-26 Colon Carcinoma Challenge

5×10⁵ CT-26 cells were injected subcutaneously (SC) in 0.1 mL of serum-free DMEM in the lower right dorsal flank of each animal. Dosing was initiated 13 days following tumor implantation. Day 0 indicates the first day of dosing. All antibodies were administered intraperitoneally (IP) at 10 mg/kg on Days 0, 4, 8, and 12. Animals were weighed and tumors were measured twice a week. Animals that remained on the study on Day 16 were euthanized.

6.1.5.2 MBT-2 Bladder Carcinoma Challenge

5×10⁵ MBT-2 cells were injected SC in 0.1 mL of serum-free DMEM in the lower right dorsal flank of each animal. Dosing was initiated 15 days following tumor implantation. Day 0 indicates the first day of dosing. Anti-mouse PD-1 antibody muDX400 was dosed at 5 mg/kg, and the other antibodies were dosed at 10 mg/kg. With the exception of animals that were euthanized or found dead prior to Day 16, antibodies were administered IP on Days 0, 4, 8, 12, and 16. Animals were weighed and tumors were measured twice a week. Animals that remained on the study on Day 30 were euthanized.

6.1.5.3 RENCA Renal Carcinoma Challenge

1×10⁶ RENCA cells were injected SC in 0.1 mL of serum-free RPMI 1640 in the lower right dorsal flank of each animal. Dosing was initiated 12 days following tumor implantation. Day 0 indicates the first day of dosing. Anti-mouse PD-1 antibody muDX400 was dosed at 5 mg/kg, and the other antibodies were dosed at 10 mg/kg. With the exception of animals that were euthanized or found dead prior to Day 23 or were not dosed as scheduled due to body weight loss exceeding 15%, antibodies were administered IP on Days 0, 4, 8, 12, 16, and 23. Animals that were given a “dosing holiday” and remained on study to Day 23 were administered a “make-up” dose following recovery in body weight. Animals were weighed and tumors were measured twice a week. Animals that remained on the study on Day 49 were euthanized.

6.1.6 Statistical Methods

Comparison of tumor volumes between treatments were made at each day of follow-up, and also collectively over all time points using area under the curve (AUC) as a summary measure for each tumor.

6.1.6.1 Analysis of Tumor Volumes by Day

Follow-up of individual animals could be terminated early because of excessive tumor burden or other reasons. Depending on the reason and tumor size at the last measurement, the last observed tumor volume was treated as a lower bound on volume at all later days for that animal (right-censored data).

To compare two treatment groups on a given day, the Peto & Peto version of the Gehan-Breslow test for right-censored data was used (Klein and Moeschberger, Survival Analysis, 2^{nd} ed. Springer (2003)). In the absence of censoring, this reduces to the nonparametric Mann-Whitney (or Wilcoxon rank sum) test. Two-sided p-values were estimated from 20,000 random reassignments of animals between the two treatment groups being compared. To control the familywise error rate across all time points for a given pair of treatment groups, p-values were multiplicity adjusted by Holm's method. A p-value of less than 0.05 was used to define statistical significance.

For descriptive purposes, volumes for each day and treatment group were summarized by their median. To allow for censoring, a distribution function for each day and treatment group was estimated by the Kaplan-Meier method, with confidence band obtained using the beta product confidence procedure (Fay et al., Biostatistics, 14:723-736 (2013)). The median was estimated as the 50th percentile of the distribution function, with confidence interval obtained by inverting the confidence band. A 68% confidence level was used, to be comparable to the common “mean±SE” format for summarizing data, since the latter is approximately a 68% confidence interval for the mean.

When follow-up of an animal was terminated early, the reason was categorized and the animal's data were handled as follows: (1) tumor burden: right-censor at last measured value; (2) tumor ulceration; found dead, metastases found; found dead, unknown reason; protocol deviation; administrative; accident: right-censor at last measured value, provided this exceeded a threshold (1000 mm³); otherwise omit animal at later times; (3) weight loss/ill; acute treatment toxicity; found dead, with evidence of illness: omit animal at later times.

6.1.6.2 Analysis of AUC

For each tumor, area under the tumor volume curve (AUC) was calculated from baseline to the last measurement day for that animal, using linear interpolation between measurement times. This produced a single summary value for each tumor. However as noted above, follow-up times could differ between animals and therefore their AUC values were not directly comparable. To compare treatment groups with respect to AUC while allowing for right-censoring and dropouts, the Wilcoxon-type nonparametric test proposed by Vardi et al., Biometrika, 88:949-960 (2001) was used. P-values were obtained by the same random permutation procedure as used for by-day analyses. Because AUC combines data over time, no multiplicity adjustment across time points was needed.

6.1.7 Treatment Results

6.1.7.1 CT-26 Colon Carcinoma Challenge Results

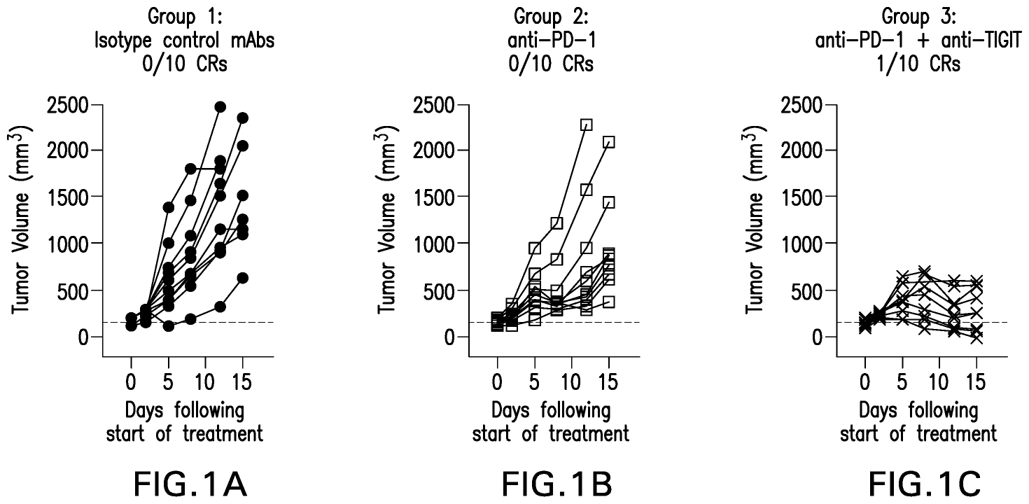

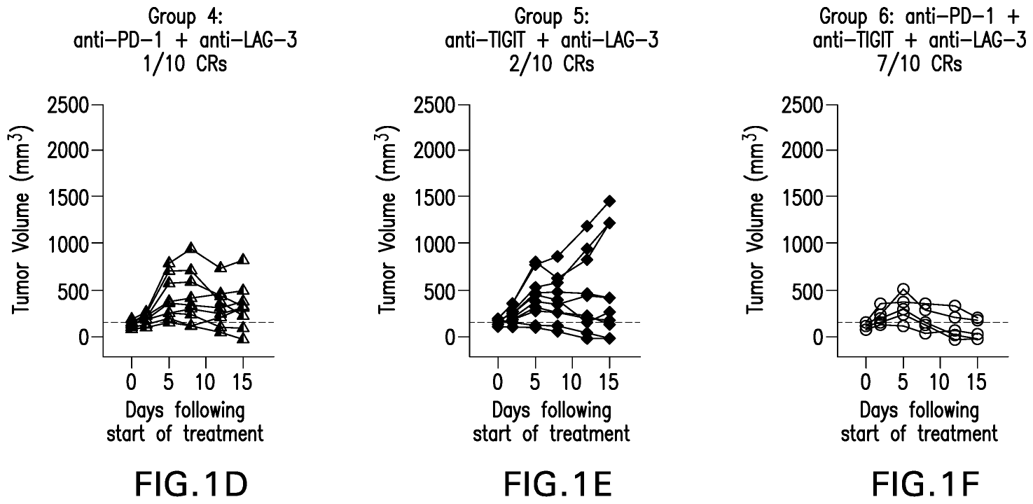

CT-26 tumor-bearing BALB/c mice were grouped into six treatment groups on Day 0 when the mean volume of tumors reached approximately 166 mm³ (109 mm³-221 mm³): (1) mIgG1 D265A isotype control+mIgG2a isotype control; (2) anti-mPD-1 mIgG1 D265A+mIgG2a isotype control; (3) anti-mPD-1 mIgG1 D265A+anti-TIGIT mIgG2a; (4) anti-PD-1 mIgG1 D265A+anti-LAG-3 mIgG1 D265A; and (5) anti-TIGIT mIgG2a+anti-LAG-3 mIgG1 D265A; and (6) anti-PD-1 mIgG1 D265A+anti-TIGIT mIgG2a+anti-LAG-3 mIgG1 D265A. Complete regression (CR) of a tumor was defined as the absence of a measurable tumor or either the length or width not exceeding 3 mm. The results are shown in FIGS. 1A-1F. Blockade of all three checkpoint inhibitors PD-1, TIGIT, and LAG3 resulted in 7 CRs (FIG. 1F), whereas blockade of PD-1 and TIGIT resulted in 1 CR (FIG. 1C), blockade of PD-1 and LAG3 resulted in 1 CR (FIG. 1D), and blockade of LAG-3 and TIGIT resulted in 2 CRs (FIG. 1E).

Compared to blockade of two of the three checkpoint inhibitors (Groups 3, 4, or 5), a cocktail of anti-PD-1+anti-TIGIT+anti-LAG3 (Group 6) resulted in significantly reduced tumor volumes by Day 15 (p<0.05, multiplicity adjusted across time points) (Table 5). By AUC analysis, a cocktail of anti-PD-1+anti-TIGIT+anti-LAG3 (Group 6) resulted in significantly reduced tumor compared to blockade of PD-1 and TIGIT (Group 3) or blockade of PD-1 and LAG3 (Group 4) (Table 6).

TABLE 5
Unadjusted and Multiplicity-adjusted P-values for Comparison of Tumor Volumes

| | Groups being compared | | Day 2 | Day 5 | Day 8 | Day 12 | Day 15 |
|---|---|---|---|---|---|---|---|
| Unadjusted | anti-PD-1 + | anti-PD-1 + | 0.16415 | 0.06005 | 0.01350 | 0.00770 | 0.00160 |
| p-value* | anti-LAG3 + | anti-TIGIT | | | | | |
| | anti-TIGIT | | | | | | |
| | anti-PD-1 + | anti-PD-1 + | 0.79715 | 0.27385 | 0.05215 | 0.00250 | 0.00060 |
| | anti-LAG3 + | anti-LAG3 | | | | | |
| | anti-TIGIT | | | | | | |
| | anti-PD-1 + | anti-TIGIT + | 0.57945 | 0.12240 | 0.03555 | 0.02515 | 0.00840 |
| | anti-LAG3 + | anti-LAG3 | | | | | |
| | anti-TIGIT | | | | | | |
| Multiplicity- | anti-PD-1 + | anti-PD-1 + | 0.16415 | 0.12010 | 0.04050 | 0.03080 | 0.00800 |
| adjusted | anti-LAG3 + | anti-TIGIT | | | | | |
| p-value** | anti-TIGIT | | | | | | |
| | anti-PD-1 + | anti-PD-1 + | 0.79715 | 0.54770 | 0.15645 | 0.01000 | 0.00300 |
| | anti-LAG3 + | anti-LAG3 | | | | | |
| | anti-TIGIT | | | | | | |
| | anti-PD-1 + | anti-TIGIT + | 0.57945 | 0.24480 | 0.10665 | 0.10060 | 0.04200 |
| | anti-LAG3 + | anti-LAG3 | | | | | |
| | anti-TIGIT | | | | | | |

*Two-sided p-values based on the Peto & Peto version of the Gehan-Breslow nonparametric test statistic for right-censored data. P-values were estimated from 20,000 random reassignments of animals between the two treatments being compared.
**P-values adjusted to control the familywise error rate across all time points for a given pair of treatments. Adjustment was by Holm's method.

TABLE 6
P-values for Comparison of Area under
the Tumor Volume Curve (AUC)

| | Groups being compared | | AUC |
|---|---|---|---|
| p-value* | anti-PD-1 + | anti-PD-1 + | 0.01350 |
| | anti-LAG3 + | anti-TIGIT | |
| | anti-TIGIT | | |
| | anti-PD-1 + | anti-PD-1 + | 0.01105 |
| | anti-LAG3 + | anti-LAG3 | |
| | anti-TIGIT | | |
| | anti-PD-1 + | anti-TIGIT + | 0.06435 |
| | anti-LAG3 + | anti-LAG3 | |
| | anti-TIGIT | | |

*Comparison of groups using the AUC of tumor volume for each animal. AUC for a given animal was calculated from baseline to the last measurement time for that animal.

The Wilcoxon-type nonparametric test proposed by Vardi et al (2001) was used to compare AUC's, with permutation p-values obtained as for the day-by-day analyses.

6.1.7.2 MBT-2 Bladder Carcinoma Challenge Results

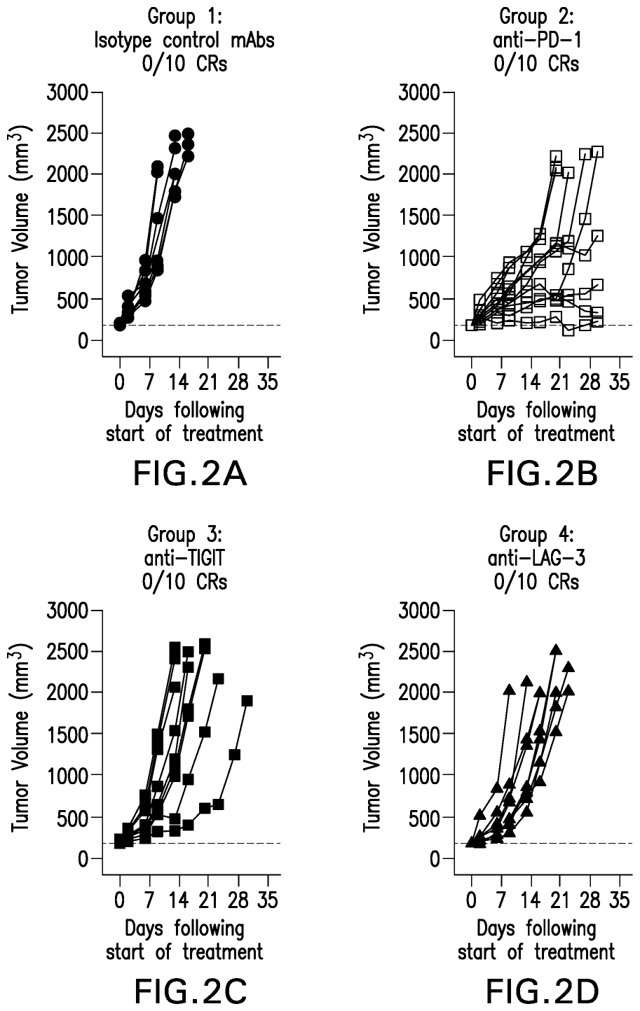

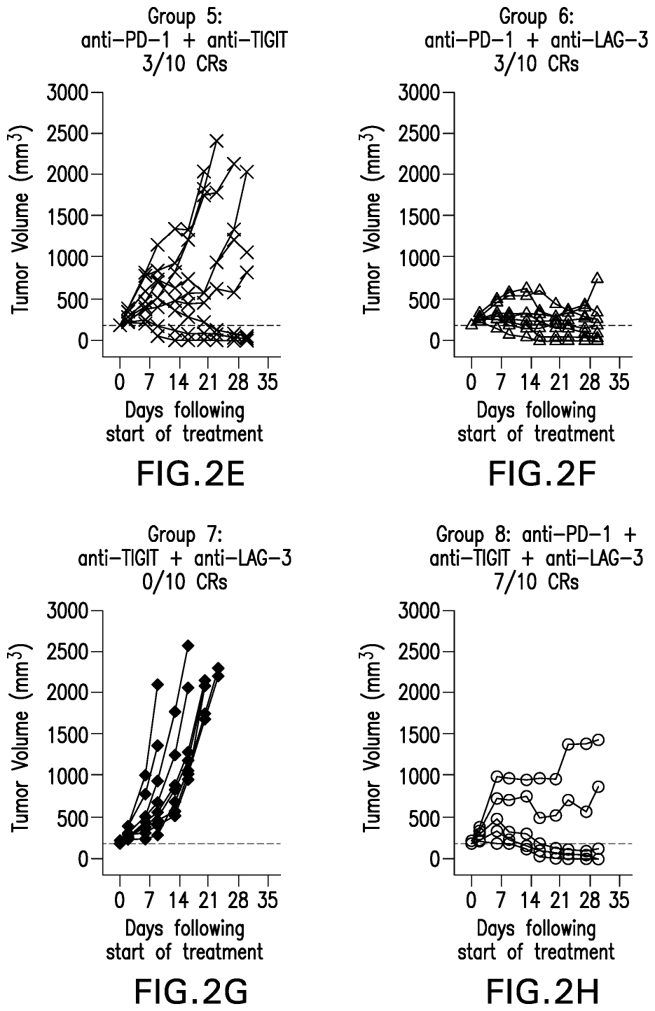

MBT-2 tumor-bearing C3H/He mice were grouped into eight treatment groups on Day 0 when the mean volume of tumors reached approximately 196 mm³(180 mm³-220 mm³): (1) mIgG1 isotype control+mIgG2a isotype control; (2) anti-mPD-1 mIgG1 D265A+mIgG2a isotype control; (3) anti-TIGIT mIgG2a+mIgG1 isotype control; (4) anti-LAG-3 mIgG1 D265A+mIgG2a isotype control; (5) anti-mPD-1 mIgG1 D265A+anti-TIGIT mIgG2a; (6) anti-PD-1 mIgG1 D265A+anti-LAG-3 mIgG1 D265A; (7) anti-TIGIT mIgG2a+anti-LAG-3 mIgG1 D265A; and (8) anti-PD-1 mIgG1 D265A+anti-TIGIT mIgG2a+anti-LAG-3 mIgG1 D265A. Complete regression (CR) of a tumor was defined as the absence of a measurable tumor. The results are shown in FIGS. 2A-2H. Blockade of all three checkpoint inhibitors PD-1, TIGIT, and LAG3 resulted in 7 CRs (FIG. 2H), whereas blockade of PD-1 and TIGIT resulted in 3 CRs (FIG. 2E), blockade of PD-1 and LAG3 resulted in 3 CRs (FIG. 2F), and blockade of LAG-3 and TIGIT resulted in 0 CR (FIG. 2G).

Compared to blockade of LAG3 and TIGIT (Group 7), a cocktail of anti-PD-1+anti-TIGIT+anti-LAG3 (Group 8) resulted in significantly reduced tumor volumes by Day 30 (p<0.05, multiplicity adjusted across time points) (Table 7) and by AUC analysis (Table 8). However, no statistically significant reduction of tumor volumes was observed when comparing blockade of all three checkpoint inhibitors (Group 8) to blockade of PD-1 and TIGIT (Group 5) or blockade of PD-1 and LAG3 (Group 6).

TABLE 7
Unadjusted and Multiplicity-adjusted P-values for Comparison of Tumor Volumes

| | Groups being compared | | Day 16 | Day 20 | Day 23 | Day 27 | Day 30 |
|---|---|---|---|---|---|---|---|
| Unadjusted | anti-PD-1 + | anti-PD-1 + | 0.15755 | 0.37925 | 0.34020 | 0.11595 | 0.02600 |
| p-value* | anti-LAG3 + | anti-TIGIT | | | | | |
| | anti-TIGIT | | | | | | |
| | anti-PD-1 + | anti-PD-1 + | 0.91090 | 0.18760 | 0.68555 | 0.30640 | 0.04155 |
| | anti-LAG3 + | anti-LAG3 | | | | | |
| | anti-TIGIT | | | | | | |
| | anti-PD-1 + | anti-TIGIT + | 0.00005 | 0.00005 | 0.00005 | 0.00005 | 0.00005 |
| | anti-LAG3 + | anti-LAG3 | | | | | |
| | anti-TIGIT | | | | | | |
| Multiplicity- | anti-PD-1 + | anti-PD-1 + | 0.47265 | 0.68040 | 0.68040 | 0.46380 | 0.13000 |
| adjusted | anti-LAG3 + | anti-TIGIT | | | | | |
| p-value** | anti-TIGIT | | | | | | |
| | anti-PD-1 + | anti-PD-1 + | 1.00000 | 0.75040 | 1.00000 | 0.91920 | 0.20775 |
| | anti-LAG3 + | anti-LAG3 | | | | | |
| | anti-TIGIT | | | | | | |
| | anti-PD-1 + | anti-TIGIT + | 0.00025 | 0.00025 | 0.00025 | 0.00025 | 0.00025 |
| | anti-LAG3 + | anti-LAG3 | | | | | |
| | anti-TIGIT | | | | | | |

*Two-sided p-values based on the Peto & Peto version of the Gehan-Breslow nonparametric test statistic for right-censored data. P-values were estimated from 20,000 random reassignments of animals between the two treatments being compared.
**P-values adjusted to control the familywise error rate across all time points for a given pair of treatments. Adjustment was by Holm's method.

TABLE 8
P-values for Comparison of Area under
the Tumor Volume Curve (AUC)

| | Groups being compared | | AUC |
|---|---|---|---|
| p-value* | anti-PD-1 + | anti-PD-1 + | 0.12075 |
| | anti-LAG3 + | anti-TIGIT | |
| | anti-TIGIT | | |
| | anti-PD-1 + | anti-PD-1 + | 0.34995 |
| | anti-LAG3 + | anti-LAG3 | |
| | anti-TIGIT | | |
| | anti-PD-1 + | anti-TIGIT + | 0.00005 |
| | anti-LAG3 + | anti-LAG3 | |
| | anti-TIGIT | | |

*Comparison of groups using the AUC of tumor volume for each animal. AUC for a given animal was calculated from baseline to the last measurement time for that animal. The Wilcoxon-type nonparametric test proposed by Vardi et al (2001) was used to compare AUC's, with permutation p-values obtained as for the day-by-day analyses.

6.1.7.3 RENCA Renal Carcinoma Challenge Results

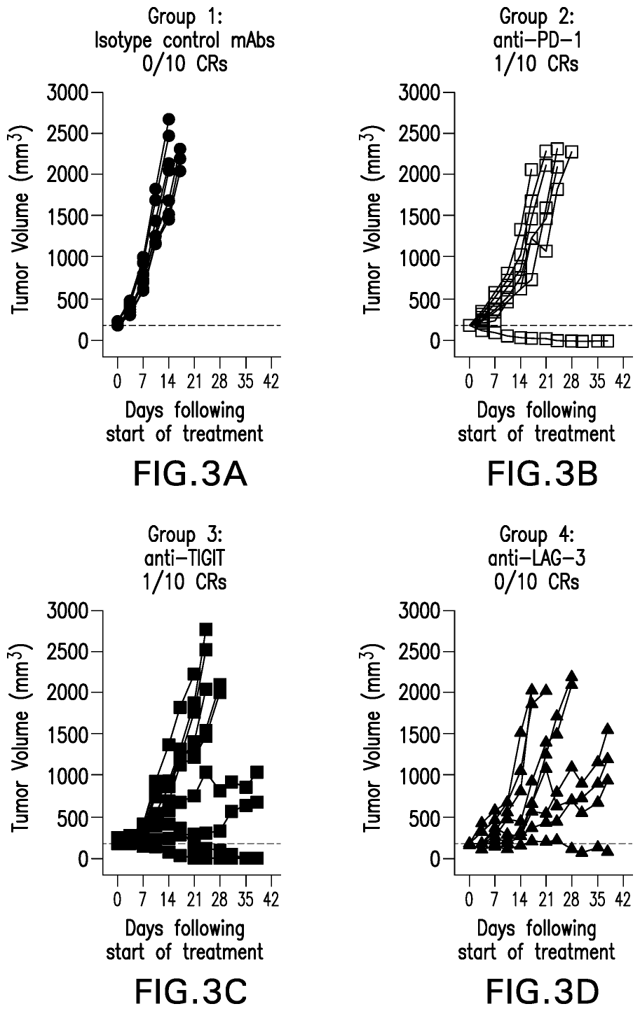

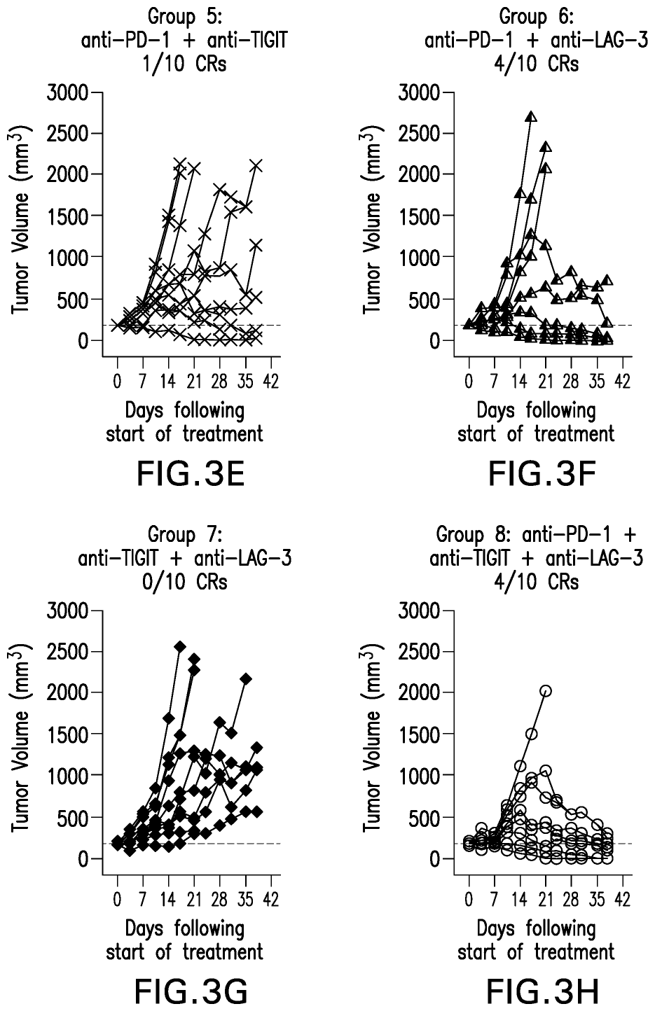

RENCA tumor-bearing BALB/c mice were grouped into eight treatment groups on Day 0 when the mean volume of tumors reached approximately 205 mm³ (172 mm³-225 mm³): (1) mIgG1 isotype control+mIgG2a isotype control; (2) anti-mPD-1 mIgG1 D265A+mIgG2a isotype control; (3) anti-TIGIT mIgG2a+mIgG1 isotype control; (4) anti-LAG-3 mIgG1 D265A+mIgG2a isotype control; (5) anti-mPD-1 mIgG1 D265A+anti-TIGIT mIgG2a; (6) anti-PD-1 mIgG1 D265A+anti-LAG-3 mIgG1 D265A; (7) anti-TIGIT mIgG2a+anti-LAG-3 mIgG1 D265A; and (8) anti-PD-1 mIgG1 D265A+anti-TIGIT mIgG2a+anti-LAG-3 mIgG1 D265A. Complete regression (CR) of a tumor was defined as the absence of a measurable tumor. The results are shown in FIGS. 3A-3H. Blockade of all three checkpoint inhibitors PD-1, TIGIT, and LAG3 resulted in 4 CRs (FIG. 3H), whereas blockade of PD-1 and TIGIT resulted in 1 CR (FIG. 3E), blockade of PD-1 and LAG3 resulted in 4 CRs (FIG. 3F), and blockade of LAG-3 and TIGIT resulted in 0 CR (FIG. 3G).

Compared to blockade of LAG3 and TIGIT (Group 7), a cocktail of anti-PD-1+anti-TIGIT+anti-LAG3 (Group 8) resulted in significantly reduced tumor volumes by Day 24 (p<0.05, multiplicity adjusted across time points) (Table 9) and by AUC analysis (Table 10). However, no statistically significant reduction of tumor volumes was observed when comparing blockade of all three checkpoint inhibitors to blockade of PD-1 and LAG3 or blockade of PD-1 and TIGIT.

TABLE 9
Unadjusted and Multiplicity-adjusted P-values for Comparison of Tumor Volumes

| | | | Day | Day | Day | Day | Day | Day | Day | Day |
|---|---|---|---|---|---|---|---|---|---|---|
| | Groups being compared | | 3 | 7 | 10 | 14 | 17 | 21 | 24 | 28 |
| Unadjusted | anti-PD-1 + | anti-PD-1 + | 0.79510 | 0.01150 | 0.08830 | 0.35475 | 0.22065 | 0.15235 | 0.07410 | 0.06070 |
| p-value* | anti-LAG3 + | anti-TIGIT | | | | | | | | |
| | anti-TIGIT | | | | | | | | | |
| | anti-PD-1 + | anti-PD-1 + | 0.85390 | 0.97015 | 0.58085 | 0.85380 | 0.74115 | 0.63600 | 0.90120 | 0.53660 |
| | anti-LAG3 + | anti-LAG3 | | | | | | | | |
| | anti-TIGIT | | | | | | | | | |
| | anti-PD-1 + | anti-TIGIT + | 0.85655 | 0.68270 | 0.39450 | 0.21825 | 0.12385 | 0.03540 | 0.00610 | 0.00280 |
| | anti-LAG3 + | anti-LAG3 | | | | | | | | |
| | anti-TIGIT | | | | | | | | | |
| Multiplicity- | anti-PD-1 + | anti-PD-1 + | 0.79510 | 0.16100 | 0.62040 | 0.70950 | 0.66195 | 0.62040 | 0.62040 | 0.62040 |
| adjusted | anti-LAG3 + | anti-TIGIT | | | | | | | | |
| p-value** | anti-TIGIT | | | | | | | | | |
| | anti-PD-1 + | anti-PD-1 + | 1.00000 | 1.00000 | 1.00000 | 1.00000 | 1.00000 | 1.00000 | 1.00000 | 1.00000 |
| | anti-LAG3 + | anti-LAG3 | | | | | | | | |
| | anti-TIGIT | | | | | | | | | |
| | anti-PD-1 + | anti-TIGIT + | 1.00000 | 1.00000 | 1.00000 | 0.87300 | 0.61925 | 0.21240 | 0.04270 | 0.02520 |
| | anti-LAG3 + | anti-LAG3 | | | | | | | | |
| | anti-TIGIT | | | | | | | | | |

| | | | Day | Day | Day | Day | Day | Day |
|---|---|---|---|---|---|---|---|---|
| | Groups being compared | | 31 | 35 | 38 | 42 | 45 | 49 |
| Unadjusted | anti-PD-1 + | anti-PD-1 + | 0.06050 | 0.08020 | 0.04255 | 0.05170 | 0.06280 | 0.05200 |
| p-value* | anti-LAG3 + | anti-TIGIT | | | | | | |
| | anti-TIGIT | | | | | | | |
| | anti-PD-1 + | anti-PD-1 + | 0.53570 | 0.50425 | 0.61280 | 0.71680 | 0.59915 | 0.71680 |
| | anti-LAG3 + | anti-LAG3 | | | | | | |
| | anti-TIGIT | | | | | | | |
| | anti-PD-1 + | anti-TIGIT + | 0.00290 | 0.00205 | 0.00205 | 0.00090 | 0.00080 | 0.00065 |
| | anti-LAG3 + | anti-LAG3 | | | | | | |
| | anti-TIGIT | | | | | | | |
| Multiplicity- | anti-PD-1 + | anti-PD-1 + | 0.62040 | 0.62040 | 0.55315 | 0.62040 | 0.62040 | 0.62040 |
| adjusted | anti-LAG3 + | anti-TIGIT | | | | | | |
| p-value** | anti-TIGIT | | | | | | | |
| | anti-PD-1 + | anti-PD-1 + | 1.00000 | 1.00000 | 1.00000 | 1.00000 | 1.00000 | 1.00000 |
| | anti-LAG3 + | anti-LAG3 | | | | | | |
| | anti-TIGIT | | | | | | | |
| | anti-PD-1 + | anti-TIGIT + | 0.02520 | 0.02255 | 0.02255 | 0.01080 | 0.01040 | 0.00910 |
| | anti-LAG3 + | anti-LAG3 | | | | | | |
| | anti-TIGIT | | | | | | | |

*Two-sided p-values based on the Peto & Peto version of the Gehan-Breslow nonparametric test statistic for right-censored data. P-values were estimated from 20,000 random reassignments of animals between the two treatments being compared.
**P-values adjusted to control the familywise error rate across all time points for a given pair of treatments. Adjustment was by Holm's method.

TABLE 10
P-values for Comparison of Area under
the Tumor Volume Curve (AUC)

| | Groups being compared | | AUC |
|---|---|---|---|
| p-value* | anti-PD-1 + | anti-PD-1 + | 0.09260 |
| | anti-LAG3 + | anti-TIGIT | |
| | anti-TIGIT | | |
| | anti-PD-1 + | anti-PD-1 + | 0.68875 |
| | anti-LAG3 + | anti-LAG3 | |
| | anti-TIGIT | | |
| | anti-PD-1 + | anti-TIGIT + | 0.01065 |
| | anti-LAG3 + | anti-LAG3 | |
| | anti-TIGIT | | |

*Comparison of groups using the AUC of tumor volume for each animal. AUC for a given animal was calculated from baseline to the last measurement time for that animal. The Wilcoxon-type nonparametric test proposed by Vardi et al (2001) was used to compare AUC's, with permutation p-values obtained as for the day-by-day analyses.

### 6.2 Example 2: A Combination of an Anti-human PD-1 Monoclonal Antibody, an Anti-human LAG3 Monoclonal Antibody, and an Anti-human TIGIT Monoclonal Antibody Enhanced IFN-γ Production and Proliferation in Human CD4+ T Cells

The effect of a triple combination of an anti-human PD-1 monoclonal antibody, an anti-human LAG3 monoclonal antibody, and an anti-human monoclonal TIGIT antibody on cytokine production (e.g., INF-γ) and cell proliferation were performed on four independently prepared batches of human CD4+ T cells.

6.2.1 Generation and Culture of Human CD4+ T Cell Clone

MHC class II alloantigen specific CD4+ T cell clone BC4-49 was generated at Merck-Palo Alto by 2 rounds of mixed leukocyte reaction with the EBV-transformed B-cell line JY and cloned by limiting dilution. The clone was re-stimulated with allospecific antigens at an interval of every 2 weeks and cultured in Yssel's medium (IMDM, Gibco by Thermo Fisher Scientific, Cat #i12440-053, Waltham, MA; human serum AB, Gemini Bio-Product, Cat #100-512, West Sacromento, CA; penicillin/streptomycin, Coming, Cat #30-002-CI, Manassas, VA; Albumin solution human, Sigma, Cat #A9080, St. Louis, MO; Insulin-Transferrin-Selenium-Ethanolamine ITS-X, Gibco by Thermo Fisher Scientific, Cat #51500056; Transferrin from human serum, Sigma, Cat #10652202001; Palmitic acid, Sigma Cat #P5585; Linoleic Acid-Oleic Acid-Albumin, Sigma, Cat #L9655). Fresh PBMCs were isolated from two human buffy coats provided by Stanford Blood Center and pooled at 1:1 cell ratio. PBMCs were irradiated in a gamma irradiator at a dose of 4000 rads before use. Wildtype JY cells were prepared and irradiated at a dose of 5000 rads. T cell clones were cultured with feeders in 24-well plates at 1 mL per well with final concentrations of CD4+ T cells at 0.2×10⁶/mL, irradiated PBMCs at 1×10⁶/mL, irradiated JY cells at 0.1×10⁶/mL, and 100 ng/mL PHA (Sigma, Cat #L9017). Recombinant human IL-2 (R&D Systems, Cat #202-IL/CF, Minneapolis, MN) was added at a final concentration of 100 ng/mL on day 3 after re-stimulation, and was replenished every 3-4 days throughout the expansion. Cells were passaged to an optimal concentration of 0.5-1.0×10⁶/mL. On day 7 after re-stimulation, abundant expression of LAG3 and TIGIT and moderate expression of PD-1 were detected on the T cell surface.

6.2.2 Human CD4+ T Cell Functional Assays

Alloantigen specific CD4+ T cells were harvested from 24-well culture plates on day 7 after antigen re-stimulation and washed twice with 20 mL PBS (Hyclone, Cat #SH3002802, Pittsburgh, PA) containing 2 mM EDTA (Invitrogen by Thermo Fisher Scientific, Cat #15575-38). The cell pellets were re-suspended into single cell suspension in Yssel's medium. Tested bivalent antibodies were titrated by 5-fold serial dilutions, starting from the highest final concentration of 67 nM (133 nM of binding sites), in 100 μL Yssel's medium in Falcon 96-well U-bottom culture plates (Coming, Cat #353077, Manassas, VA). Fifty microliters of T cell suspension at a density of 4×10′ cells/mL were added into wells containing titrated antibodies. The antibody/T cell mixture was pre-incubated for 1 hour in an incubator at 37° C. with 5% CO₂. JY cells expressing human PD-L1 and CD155 transgenes (JY.hPD-L1/CD155) were co-cultured to provide allo-specific antigens. JY.hPD-L1/CD155 cells cultured in complete RPMI medium (Cellgro RPMI by Coming, Cat #10-040-CV; Hyclone Fetal Bovine Serum, GE Healthcare, Cat #SH30071; penicilin/streptomycin, Coming, Cat #30-002-CI; Sodium Pyruvate, Coming, Cat #25-00CI; MEM Non-Essential Amino Acids, Gibco by Thermo Fisher Scientific, Cat #11140) were harvested and irradiated in a gamma irradiator at a dose of 5000 rads, then washed twice with PBS containing 2 mM EDTA. The pellet was re-suspended with Yssel's medium, and filtered with 40 μm cell strainer (Thermo Fisher Scientific, Cat #22363547) before plating. Fifty microliters per well of JY.hPD-L1/CD155 suspension at a concentration of 2×10⁵ cells/mL was dispensed into pre-incubated antibody-T cells mixture, with T cell to JY.hPD-L1/CD155 cell ratio at 2:1. All conditions were run in duplicates.

After approximately 3-day culture, 100 μL of supernatant per well was harvested for human IFN-γ quantification. Human IFN-γ ELISA was performed in pooled supernatant from duplicates by using hIFN-γ Quantikine kit (R&D Systems, Cat #SIF50, Minneapolis, MN). Assays were run following the standard protocol provided by the manufacturer. Four parameter non-linear curve fitting and EC50 values were calculated using the GraphPad prism software for each individual experiment. hIFN-γ levels from four independent experiments were further analyzed using one-way paired ANOVA, and Tukey's multiple comparison test was chosen to calculate probability values of differences between treatment groups.

The remaining cell suspension was pulsed with 1 μCi ³H-Thymidine (Perkin Elmer, Cat #NET027X005MC, Hopkinton, MA) in 10 μL PBS per well, and incubated at 37° C. with 5% CO₂ for 6 hours. Cells were then harvested to a 96-well formatted glass fibre filtermat (PerkinElmer, Cat #1450-421), and filtermat was air-dried overnight in a vented chemical hood. ³H-Thymidine incorporation was quantified using Microbeta Counter (PerkinElmer 2450 Microplate Counter). Radiation intensity CPM (counts per minute) was used as the readout for cell proliferation. Four parameter non-linear curve fitting and EC50 values were calculated using the GraphPad prism software for each individual experiment.

6.2.3 Human CD4+ T Cell Functional Assay Results

Blockade of all three checkpoint inhibitors PD-1, LAG3, and TIGIT by a combination of an anti-human PD-1 monoclonal antibody, an anti-human LAG3 monoclonal antibody, and an anti-human TIGIT monoclonal antibody allowed human CD4+ T cells to respond to the allogeneic stimulation for IFN-γ production to a greater degree, compared with any dual combinations of the antibodies, and to a much greater degree, compared with any individual antibody alone, all in a dose-dependent manner.

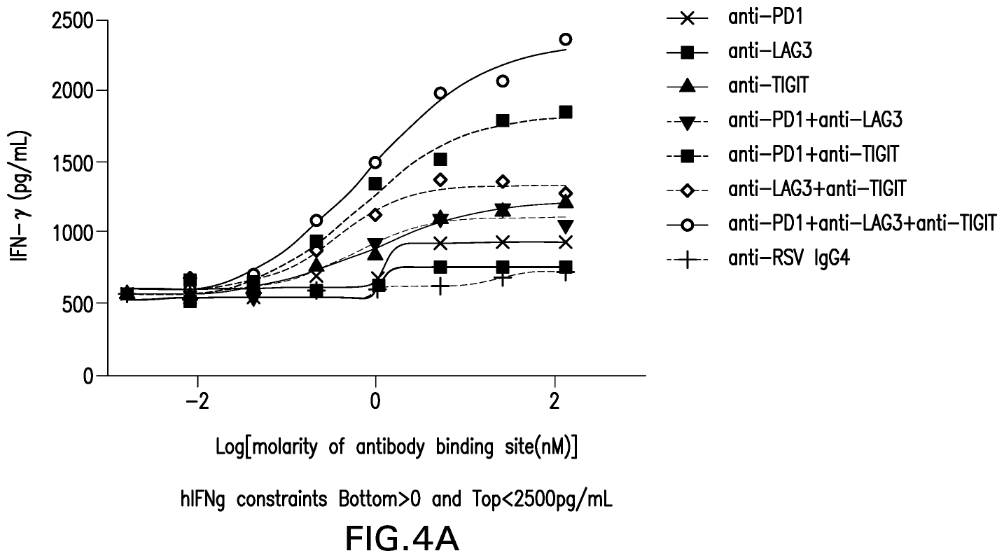

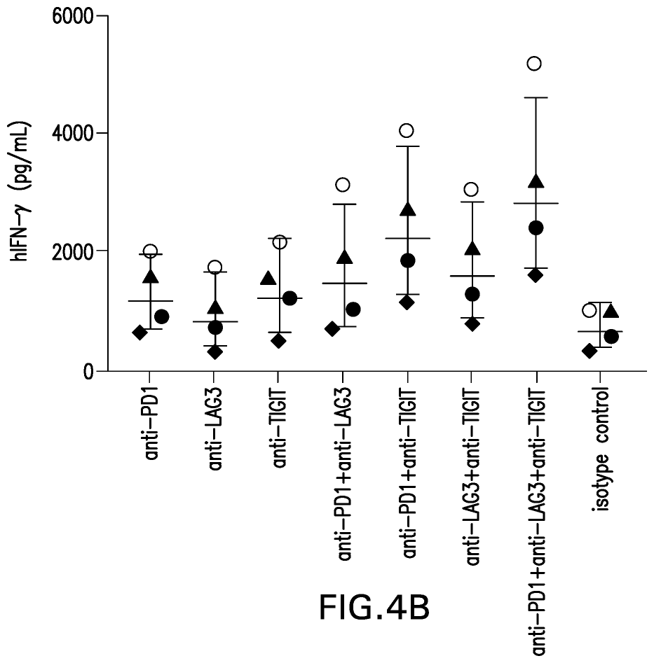

FIG. 4A shows dose-dependent curves of an exemplary assay from four independent experiments, each using a different batch of human CD4+ T cells. EC50 values of each treatment group from four independent experiments are summarized in Table 11. There was no significant difference among EC50 values of different antibodies or combinations of antibodies in inducing IFN-γ production, suggesting that the binding affinity of individual antibodies were not affected while used in combination.

TABLE 11
EC50 Values of Antibody-induced IFNγ Production in Human CD4+ T Cells

| | | | | | | | anti-PD1 + |
|---|---|---|---|---|---|---|---|
| EC50 | anti- | anti- | anti- | anti-PD1 + | anti-PD1 + | anti-LAG3 + | anti-LAG3 + |
| (nM) | PD1 | LAG3 | TIGIT | anti-LAG3 | anti-TIGIT | anti-TIGIT | anti-TIGIT |
| Experiment 1 | 1.31 | 1.19 | 1.05 | 0.54 | 0.73 | 0.38 | 0.83 |
| Experiment 2 | 4.76 | 0.12 | 0.24 | N.A. | N.A. | 0.36 | 1.36 |
| Experiment 3 | 0.50 | 0.11 | 0.30 | 0.34 | 0.74 | 0.21 | 1.02 |
| Experiment 4 | 0.37 | 0.04 | 0.58 | 0.72 | 1.38 | 0.43 | 12.19 |
| Mean | 1.73 | 0.37 | 0.54 | 0.53 | 0.95 | 0.35 | 3.85 |
| SD | 2.06 | 0.55 | 0.37 | 0.19 | 0.37 | 0.09 | 5.56 |

FIG. 4B summarizes the IFN-γ levels at 67 nM antibody concentration in the four independent experiments. Each symbol represents an individual experiment. Statistical test analyses using paired ANOVA and Turkey's multiple comparison indicate that the combination of the three antibodies induced significant increases in the IFN-γ levels, compared to individual antibodies alone or combinations of any two antibodies.

TABLE 12
IFN-γ Statistical Test Analyses

| Treatment to be compared with | Differences of |
|---|---|
| anti-PD-1 + anti-LAG3 + anti-TIGIT | IFN-γ levels |
| anti-PDl | *** |
| anti-LAG3 | **** |
| anti-TIGIT | *** |
| anti-PD1 + anti-LAG3 | ** |
| anti-PD1 + anti-TIGIT# | n.s. |
| anti-LAG3 + anti-TIGIT | ** |
| isotype control | **** |

n.s. non-significant
* P < 0.05
** P < 0.01
*** P < 0.001
**** P < 0.0001
#paired T test P < 0.05

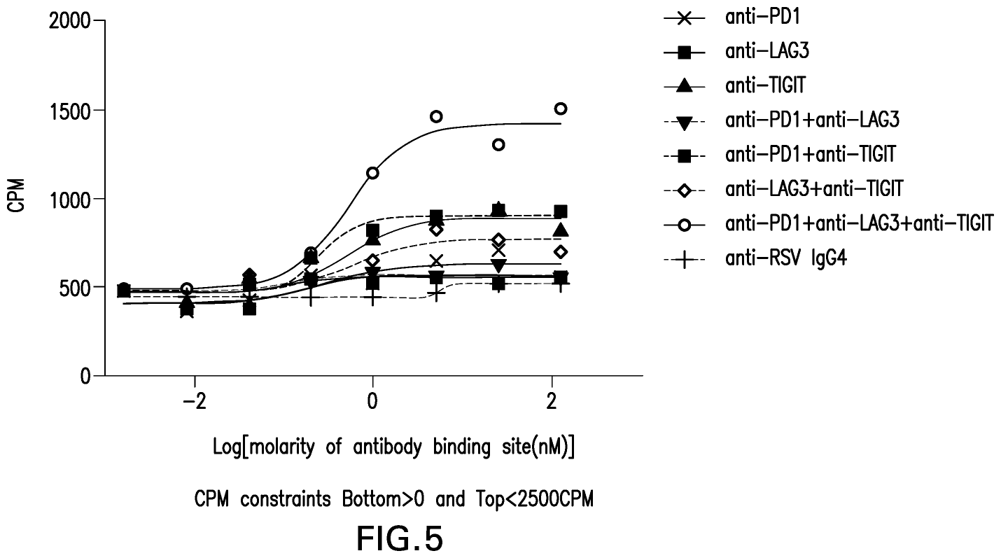

Similarly, blockade of all three checkpoint inhibitors PD-1, LAG3, and TIGIT resulted in enhanced cell proliferation (indicated by ³H-Thymidine incorporation) in human CD4+ T cells upon allogeneic stimulation, compared to blockade of any one or two of the three checkpoint inhibitors (FIG. 5).

### VII. SEQUENCE LISTING

The present specification is being filed with a computer readable form (CRF) copy of the Sequence Listing. The CRF entitled 24563_WO_PCT_SEQLIST.txt, which was created on Jan. 8, 2019 and is 90,328 bytes in size, is incorporated herein by reference in its entirety.

Table 13 below summarizes all sequences disclosed in the present specification.

TABLE 13
SEQ ID NOS and Corresponding Sequences

| SEQ | | |
|---|---|---|
| ID | | |
| NO | Description | Sequence |
| 1 | Pembrolizumab, | RASKGVSTSGYSYLH |
| | VL-CDR1 | |
| 2 | Pembrolizumab, | LASYLES |
| | VL-CDR2 | |
| 3 | Pembrolizumab, | QHSRDLPLT |
| | VL-CDR3 | |
| 4 | Pembrolizumab, | EIVLTQSPATLSLSPGERATLSCRASKGVSTSGYSYLHWYQQKPGQAPRL |
| | VL | LIYLASYLESGVPARFSGSGSGTDFTLTISSLEPEDFAVYYCQHSRDLPL |
| | | TFGGGTKVEIK |
| 5 | Pembrolizumab, | EIVLTQSPATLSLSPGERATLSCRASKGVSTSGYSYLHWYQQKPGQAPRL |
| | light chain | LIYLASYLESGVPARFSGSGSGTDFTLTISSLEPEDFAVYYCQHSRDLPL |
| | | TFGGGTKVEIKRTVAAPSVFIFPPSDEQLKSGTASVVCLLNNFYPREAKV |
| | | QWKVDNALQSGNSQESVTEQDSKDSTYSLSSTLTLSKADYEKHKVYACEV |
| | | THQGLSSPVTKSFNRGEC |
| 6 | Pembrolizumab, | NYYMY |
| | VH-CDR1 | |
| 7 | Pembrolizumab, | GINPSNGGTNFNEKFKN |
| | VH-CDR2 | |
| 8 | Pembrolizumab, | RDYRFDMGFDY |
| | VH-CDR3 | |
| 9 | Pembrolizumab, | QVQLVQSGVEVKKPGASVKVSCKASGYTFTNYYMYWVRQAPGQGLEWMGG |
| | | INPSNGGTNFNEKFKNRVTLTTDSSTTTAYMELKSLQFDDTAVYYCARRD |
| | | VHYRFDMGFDYWGQGTTVTVSS |
| 10 | Pembrolizumab, | QVQLVQSGVEVKKPGASVKVSCKASGYTFTNYYMYWVRQAPGQGLEWMGG |
| | heavy chain | INPSNGGTNENEKEKNRVTLTTDSSTTTAYMELKSLQFDDTAVYYCARRD |
| | | YRFDMGFDYWGQGTTVTVSSASTKGPSVFPLAPCSRSTSESTAALGCLVK |
| | | DYFPEPVTVSWNSGALTSGVHTFPAVLQSSGLYSLSSVVTVPSSSLGTKT |
| | | YTCNVDHKPSNTKVDKRVESKYGPPCPPCPAPEFLGGPSVFLFPPKPKDT |
| | | LMISRTPEVTCVVVDVSQEDPEVQFNWYVDGVEVHNAKTKPREEQFNSTY |
| | | RVVSVLTVLHQDWLNGKEYKCKVSNKGLPSSIEKTISKAKGQPREPQVYT |
| | | LPPSQEEMTKNQVSLTCLVKGFYPSDIAVEWESNGQPENNYKTTPPVLDS |
| | | DGSFFLYSRLTVDKSRWQEGNVFSCSVMHEALHNHYTQKSLSLSLGK |
| 11 | Anti-LAG3 | KASQSLDYEGDSDMN |
| | Ab6, VL-CDR1 | |
| 12 | Anti-LAG3 | GASNLES |
| | Ab6, VL-CDR2 | |
| 13 | Anti-LAG3 | QQSTEDPRT |
| | Ab6, VL-CDR3 | |
| 14 | Anti-LAG3 | DIVMTQTPLSLSVTPGQPASISCKASQSLDYEGDSDMNWYLQKPGQPPQL |
| | Ab6, VL | LIYGASNLESGVPDRFSGSGSGTDFTLKISRVEAEDVGVYYCQQSTEDPR |
| | | TFGGGTKVEIK |
| 15 | Anti-LAG3 | DIVMTQTPLSLSVTPGQPASISCKASQSLDYEGDSDMNWYLQKPGQPPQL |
| | Ab6, light | LIYGASNLESGVPDRFSGSGSGTDFTLKISRVEAEDVGVYYCQQSTEDPR |
| | chain | TFGGGTKVEIKRTVAAPSVFIFPPSDEQLKSGTASVVCLLNNFYPREAKV |
| | | QWKVDNALQSGNSQESVTEQDSKDSTYSLSSTLTLSKADYEKHKVYACEV |
| | | THQGLSSPVTKSFNRGEC |
| 16 | Anti-LAG3 | DYNVD |
| | Ab6, VH-CDR1 | |
| 17 | Anti-LAG3 | DINPNDGGTIYAQKFQE |
| | Ab6, VH-CDR2 | |
| 18 | Anti-LAG3 | NYRWFGAMDH |
| | Ab6, VH-CDR3 | |
| 19 | Anti-LAG3 | QMQLVQSGPEVKKPGTSVKVSCKASGYTFTDYNVDWVRQARGQRLEWIGD |
| | Ab6, VH | INPNDGGTIYAQKFQERVTITVDKSTSTAYMELSSLRSEDTAVYYCARNY |
| | | RWFGAMDHWGQGTTVTVSS |
| 20 | Anti-LAG3 | QMQLVQSGPEVKKPGTSVKVSCKASGYTFTDYNVDWVRQARGQRLEWIGD |
| | Ab6, heavy | INPNDGGTIYAQKFQERVTITVDKSTSTAYMELSSLRSEDTAVYYCARNY |
| | chain | RWFGAMDHWGQGTTVTVSSASTKGPSVFPLAPCSRSTSESTAALGCLVKD |
| | | YFPEPVTVSWNSGALTSGVHTFPAVLQSSGLYSLSSVVTVPSSSLGTKTY |
| | | TCNVDHKPSNTKVDKRVESKYGPPCPPCPAPEFLGGPSVFLFPPKPKDTL |
| | | MISRTPEVTCVVVDVSQEDPEVQFNWYVDGVEVHNAKTKPREEQFNSTYR |
| | | VVSVLTVLHQDWLNGKEYKCKVSNKGLPSSIEKTISKAKGQPREPQVYTL |
| | | PPSQEEMTKNQVSLTCLVKGFYPSDIAVEWESNGQPENNYKTTPPVLDSD |
| | | GSFFLYSRLTVDKSRWQEGNVFSCSVMHEALHNHYTQKSLSLSLGK |
| 21 | Anti-TIGIT | RASEHIYSYLS |
| | antibody, VL- | |
| | CDR1 | |
| 22 | Anti-TIGIT | NAKTLAE |
| | antibody, VL- | |
| | CDR2 | |
| 23 | Anti-TIGIT | QHHFGSPLT |
| | antibody, VL- | |
| | CDR3 | |
| 24 | Anti-TIGIT | DIQMTQSPSSLSASVGDRVTITCRASEHIYSYLSWYQQKPGKVPKLLIYN |
| | antibody, VL | AKTLAEGVPSRFSGSGSGTDFTLTISSLQPEDVATYYCQHHFGSPLTFGQ |
| | | GTRLEIK |
| 25 | Anti-TIGIT | DIQMTQSPSSLSASVGDRVTITCRASEHIYSYLSWYQQKPGKVPKLLIYN |
| | antibody, light | AKTLAEGVPSRFSGSGSGTDFTLTISSLQPEDVATYYCQHHFGSPLTFGQ |
| | chain | GTRLEIKRTVAAPSVFIFPPSDEQLKSGTASVVCLLNNEYPREAKVQWKV |
| | | DNALQSGNSQESVTEQDSKDSTYSLSSTLTLSKADYEKHKVYACEVTHQG |
| | | LSSPVTKSFNRGEC |
| 26 | Anti-TIGIT | SYVMH |
| | antibody, VH- | |
| | CDR1 | |
| 27 | Anti-TIGIT | YIDPYNDGAKYAQKFQG |
| | antibody, VH- | |
| | CDR2 | |
| 28 | Anti-TIGIT | GGPYGWYFDV |
| | antibody, VH- | |
| | CDR3 | |
| 29 | Anti-TIGIT | EVQLVQSGAEVKKPGSSVKVSCKASGYTFSSYVMHWVRQAPGQGLEWIGY |
| | antibody, VH | IDPYNDGAKYAQKFQGRVTLTSDKSTSTAYMELSSLRSEDTAVYYCARGG |
| | | PYGWYFDVWGQGTTVTVSS |
| 30 | Anti-TIGIT | EVQLVQSGAEVKKPGSSVKVSCKASGYTFSSYVMHWVRQAPGQGLEWIGY |
| | antibody, heavy | IDPYNDGAKYAQKFQGRVTLTSDKSTSTAYMELSSLRSEDTAVYYCARGG |
| | chain | PYGWYFDVWGQGTTVTVSSASTKGPSVFPLAPSSKSTSGGTAALGCLVKD |
| | | YFPEPVTVSWNSGALTSGVHTFPAVLQSSGLYSLSSVVTVPSSSLGTQTY |
| | | ICNVNHKPSNTKVDKKVEPKSCDKTHTCPPCPAPELLGGPSVFLFPPKPK |
| | | DTLMISRTPEVTCVVVDVSHEDPEVKFNWYVDGVEVHNAKTKPREEQYNS |
| | | TYRVVSVLTVLHQDWLNGKEYKCKVSNKALPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGK |
| 31 | Nivolumab, | RASQSVSSYLA |
| | VL-CDR1 | |
| 32 | Nivolumab, | DASNRAT |
| | VL-CDR2 | |
| 33 | Nivolumab, | QQSSNWPRT |
| | VL-CDR3 | |
| 34 | Nivolumab, VL | EIVLTQSPATLSLSPGERATLSCRASQSVSSYLAWYQQKPGQAPRLLIYD |
| | | ASNRATGIPARESGSGSGTDFTLTISSLEPEDFAVYYCQQSSNWPRTFGQ |
| | | GTKVEIK |
| 35 | Nivolumab, | EIVLTQSPATLSLSPGERATLSCRASQSVSSYLAWYQQKPGQAPRLLIYD |
| | light chain | ASNRATGIPARESGSGSGTDFTLTISSLEPEDFAVYYCQQSSNWPRTFGQ |
| | | GTKVEIKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTHQG |
| | | LSSPVTKSFNRGEC |
| 36 | Nivolumab, | NSGMH |
| | VH-CDR1 | |
| 37 | Nivolumab, | VIWYDGSKRYYADSVKG |
| | VH-CDR2 | |
| 38 | Nivolumab, | NDDY |
| | VH-CDR3 | |
| 39 | Nivolumab, VH | QVQLVESGGGVVQPGRSLRLDCKASGITESNSGMHWVRQAPGKGLEWVAV |
| | | IWYDGSKRYYADSVKGRFTISRDNSKNTLFLQMNSLRAEDTAVYYCATND |
| | | DYWGQGTLVTVSS |
| 40 | Nivolumab, | QVQLVESGGGVVQPGRSLRLDCKASGITFSNSGMHWVRQAPGKGLEWVAV |
| | heavy chain | IWYDGSKRYYADSVKGRFTISRDNSKNTLFLQMNSLRAEDTAVYYCATND |
| | | DYWGQGTLVTVSSASTKGPSVFPLAPCSRSTSESTAALGCLVKDYFPEPV |
| | | TVSWNSGALTSGVHTFPAVLQSSGLYSLSSVVTVPSSSLGTKTYTCNVDH |
| | | KPSNTKVDKRVESKYGPPCPPCPAPEFLGGPSVFLFPPKPKDTLMISRTP |
| | | EVTCVVVDVSQEDPEVQFNWYVDGVEVHNAKTKPREEQFNSTYRVVSVLT |
| | | VLHQDWLNGKEYKCKVSNKGLPSSIEKTISKAKGQPREPQVYTLPPSQEE |
| | | MTKNQVSLTCLVKGFYPSDIAVEWESNGQPENNYKTTPPVLDSDGSFFLY |
| | | SRLTVDKSRWQEGNVFSCSVMHEALHNHYTQKSLSLSLGK |
| 41 | Anti-LAG3 | DINPNGGGTIYAQKFQE |
| | Ab9, VH-CDR2 | |
| 42 | Anti-LAG3 | DINPNQGGTIYAQKFQE |
| | Ab8, VH-CDR2 | |
| 43 | Anti-LAG3 | DINPNSGGTIYAQKFQE |
| | Ab7, VH-CDR2 | |
| 44 | Anti-LAG3 | DINPNNGGTIYAQKFQE |
| | Ab5, VH-CDR2 | |
| 45 | Anti-LAG3 | QMQLVQSGPEVKKPGTSVKVSCKASGYTFTDYNVDWVRQARGQRLEWIGD |
| | Ab9, VH | INPNGGGTIYAQKFQERVTITVDKSTSTAYMELSSLRSEDTAVYYCARNY |
| | | RWFGAMDHWGQGTTVTVSS |
| 46 | Anti-LAG3 | QMQLVQSGPEVKKPGTSVKVSCKASGYTFTDYNVDWVRQARGQRLEWIGD |
| | Ab8, VH | INPNQGGTIYAQKFQERVTITVDKSTSTAYMELSSLRSEDTAVYYCARNY |
| | | RWFGAMDHWGQGTTVTVSS |
| 47 | Anti-LAG3 | QMQLVQSGPEVKKPGTSVKVSCKASGYTFTDYNVDWVRQARGQRLEWIGD |
| | Ab7, VH | INPNSGGTIYAQKFQERVTITVDKSTSTAYMELSSLRSEDTAVYYCARNY |
| | | RWFGAMDHWGQGTTVTVSS |
| 48 | Anti-LAG3 | QMQLVQSGPEVKKPGTSVKVSCKASGYTFTDYNVDWVRQARGQRLEWIGD |
| | Ab5, VH | INPNNGGTIYAQKFQERVTITVDKSTSTAYMELSSLRSEDTAVYYCARNY |
| | | RWFGAMDHWGQGTTVTVSS |
| 49 | Anti-LAG3 | QMQLVQSGPEVKKPGTSVKVSCKASGYTFTDYNVDWVRQARGQRLEWIGD |
| | variant, IgG1 | INPNGGGTIYAQKFQERVTITVDKSTSTAYMELSSLRSEDTAVYYCARNY |
| | HC with VH- | RWFGAMDHWGQGTTVTVSSASTKGPSVFPLAPSSKSTSGGTAALGCLVKD |
| | CDR2 of Ab9 | YFPEPVTVSWNSGALTSGVHTFPAVLQSSGLYSLSSVVTVPSSSLGTQTY |
| | | ICNVNHKPSNTKVDKKVEPKSCDKTHTCPPCPAPELLGGPSVFLFPPKPK |
| | | DTLMISRTPEVTCVVVDVSHEDPEVKFNWYVDGVEVHNAKTKPREEQYNS |
| | | TYRVVSVLTVLHQDWLNGKEYKCKVSNKALPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGK |
| 50 | Anti-LAG3 | QMQLVQSGPEVKKPGTSVKVSCKASGYTFTDYNVDWVRQARGQRLEWIGD |
| | Ab4, IgG1 HC | INPNQGGTIYAQKFQERVTITVDKSTSTAYMELSSLRSEDTAVYYCARNY |
| | | RWFGAMDHWGQGTTVTVSSASTKGPSVFPLAPSSKSTSGGTAALGCLVKD |
| | | YFPEPVTVSWNSGALTSGVHTFPAVLQSSGLYSLSSVVTVPSSSLGTQTY |
| | | ICNVNHKPSNTKVDKKVEPKSCDKTHTCPPCPAPELLGGPSVFLFPPKPK |
| | | DTLMISRTPEVTCVVVDVSHEDPEVKFNWYVDGVEVHNAKTKPREEQYNS |
| | | TYRVVSVLTVLHQDWLNGKEYKCKVSNKALPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGK |
| 51 | Anti-LAG3 | QMQLVQSGPEVKKPGTSVKVSCKASGYTFTDYNVDWVRQARGQRLEWIGD |
| | Ab2, IgG1 HC | INPNSGGTIYAQKFQERVTITVDKSTSTAYMELSSLRSEDTAVYYCARNY |
| | | RWFGAMDHWGQGTTVTVSSASTKGPSVFPLAPSSKSTSGGTAALGCLVKD |
| | | YFPEPVTVSWNSGALTSGVHTFPAVLQSSGLYSLSSVVTVPSSSLGTQTY |
| | | ICNVNHKPSNTKVDKKVEPKSCDKTHTCPPCPAPELLGGPSVFLFPPKPK |
| | | DTLMISRTPEVTCVVVDVSHEDPEVKFNWYVDGVEVHNAKTKPREEQYNS |
| | | TYRVVSVLTVLHQDWLNGKEYKCKVSNKALPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGK |
| 52 | Anti-LAG3 | QMQLVQSGPEVKKPGTSVKVSCKASGYTFTDYNVDWVRQARGQRLEWIGD |
| | Ab3, IgG1 HC | INPNDGGTIYAQKFQERVTITVDKSTSTAYMELSSLRSEDTAVYYCARNY |
| | | RWFGAMDHWGQGTTVTVSSASTKGPSVFPLAPSSKSTSGGTAALGCLVKD |
| | | YFPEPVTVSWNSGALTSGVHTFPAVLQSSGLYSLSSVVTVPSSSLGTQTY |
| | | ICNVNHKPSNTKVDKKVEPKSCDKTHTCPPCPAPELLGGPSVFLFPPKPK |
| | | DTLMISRTPEVTCVVVDVSHEDPEVKFNWYVDGVEVHNAKTKPREEQYNS |
| | | TYRVVSVLTVLHQDWLNGKEYKCKVSNKALPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGK |
| 53 | Anti-LAG3 | QMQLVQSGPEVKKPGTSVKVSCKASGYTFTDYNVDWVRQARGQRLEWIGD |
| | Ab1, IgG1 HC | INPNNGGTIYAQKFQERVTITVDKSTSTAYMELSSLRSEDTAVYYCARNY |
| | | RWFGAMDHWGQGTTVTVSSASTKGPSVFPLAPSSKSTSGGTAALGCLVKD |
| | | YFPEPVTVSWNSGALTSGVHTFPAVLQSSGLYSLSSVVTVPSSSLGTQTY |
| | | ICNVNHKPSNTKVDKKVEPKSCDKTHTCPPCPAPELLGGPSVFLFPPKPK |
| | | DTLMISRTPEVTCVVVDVSHEDPEVKFNWYVDGVEVHNAKTKPREEQYNS |
| | | TYRVVSVLTVLHQDWLNGKEYKCKVSNKALPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGK |
| 54 | Anti-LAG3 | QMQLVQSGPEVKKPGTSVKVSCKASGYTFTDYNVDWVRQARGQRLEWIGD |
| | Ab9, IgG4- | INPNGGGTIYAQKFQERVTITVDKSTSTAYMELSSLRSEDTAVYYCARNY |
| | S228P HC | RWFGAMDHWGQGTTVTVSSASTKGPSVFPLAPCSRSTSESTAALGCLVKD |
| | | YFPEPVTVSWNSGALTSGVHTFPAVLQSSGLYSLSSVVTVPSSSLGTKTY |
| | | TCNVDHKPSNTKVDKRVESKYGPPCPPCPAPEFLGGPSVFLFPPKPKDTL |
| | | MISRTPEVTCVVVDVSQEDPEVQFNWYVDGVEVHNAKTKPREEQFNSTYR |
| | | VVSVLTVLHQDWLNGKEYKCKVSNKGLPSSIEKTISKAKGQPREPQVYTL |
| | | PPSQEEMTKNQVSLTCLVKGFYPSDIAVEWESNGQPENNYKTTPPVLDSD |
| | | GSFFLYSRLTVDKSRWQEGNVFSCSVMHEALHNHYTQKSLSLSLGK |
| 55 | Anti-LAG3 | QMQLVQSGPEVKKPGTSVKVSCKASGYTFTDYNVDWVRQARGQRLEWIGD |
| | Ab8, IgG4- | INPNQGGTIYAQKFQERVTITVDKSTSTAYMELSSLRSEDTAVYYCARNY |
| | S228P HC | RWFGAMDHWGQGTTVTVSSASTKGPSVFPLAPCSRSTSESTAALGCLVKD |
| | | YFPEPVTVSWNSGALTSGVHTFPAVLQSSGLYSLSSVVTVPSSSLGTKTY |
| | | TCNVDHKPSNTKVDKRVESKYGPPCPPCPAPEFLGGPSVFLFPPKPKDTL |
| | | MISRTPEVTCVVVDVSQEDPEVQFNWYVDGVEVHNAKTKPREEQFNSTYR |
| | | VVSVLTVLHQDWLNGKEYKCKVSNKGLPSSIEKTISKAKGQPREPQVYTL |
| | | PPSQEEMTKNQVSLTCLVKGFYPSDIAVEWESNGQPENNYKTTPPVLDSD |
| | | GSFFLYSRLTVDKSRWQEGNVFSCSVMHEALHNHYTQKSLSLSLGK |
| 56 | Anti-LAG3 | QMQLVQSGPEVKKPGTSVKVSCKASGYTFTDYNVDWVRQARGQRLEWIGD |
| | Ab7, IgG4- | INPNSGGTIYAQKFQERVTITVDKSTSTAYMELSSLRSEDTAVYYCARNY |
| | S228P HC | RWFGAMDHWGQGTTVTVSSASTKGPSVFPLAPCSRSTSESTAALGCLVKD |
| | | YFPEPVTVSWNSGALTSGVHTFPAVLQSSGLYSLSSVVTVPSSSLGTKTY |
| | | TCNVDHKPSNTKVDKRVESKYGPPCPPCPAPEFLGGPSVFLFPPKPKDTL |
| | | MISRTPEVTCVVVDVSQEDPEVQFNWYVDGVEVHNAKTKPREEQFNSTYR |
| | | VVSVLTVLHQDWLNGKEYKCKVSNKGLPSSIEKTISKAKGQPREPQVYTL |
| | | PPSQEEMTKNQVSLTCLVKGFYPSDIAVEWESNGQPENNYKTTPPVLDSD |
| | | GSFFLYSRLTVDKSRWQEGNVFSCSVMHEALHNHYTQKSLSLSLGK |
| 57 | Anti-LAG3 | QMQLVQSGPEVKKPGTSVKVSCKASGYTFTDYNVDWVRQARGQRLEWIGD |
| | Ab5, IgG4- | INPNNGGTIYAQKFQERVTITVDKSTSTAYMELSSLRSEDTAVYYCARNY |
| | S228P HC | RWFGAMDHWGQGTTVTVSSASTKGPSVFPLAPCSRSTSESTAALGCLVKD |
| | | YFPEPVTVSWNSGALTSGVHTFPAVLQSSGLYSLSSVVTVPSSSLGTKTY |
| | | TCNVDHKPSNTKVDKRVESKYGPPCPPCPAPEFLGGPSVFLFPPKPKDTL |
| | | MISRTPEVTCVVVDVSQEDPEVQFNWYVDGVEVHNAKTKPREEQFNSTYR |
| | | VVSVLTVLHQDWLNGKEYKCKVSNKGLPSSIEKTISKAKGQPREPQVYTL |
| | | PPSQEEMTKNQVSLTCLVKGFYPSDIAVEWESNGQPENNYKTTPPVLDSD |
| | | GSFFLYSRLTVDKSRWQEGNVFSCSVMHEALHNHYTQKSLSLSLGK |
| 58 | Anti-TIGIT | YIDPYNDGAKYNEKFKG |
| | antibody, VH- | |
| | CDR2 variant | |
| 59 | Anti-TIGIT | YIDPYNRGAKYNEKFG |
| | antibody, VH- | |
| | CDR2 variant | |
| 60 | Anti-TIGIT | YIDPYNLGAKYNEKFG |
| | antibody, VH- | |
| | CDR2 variant | |
| 61 | Anti-TIGIT | YIDPYNKGAKYNEKFG |
| | antibody, VH- | |
| | CDR2 variant | |
| 62 | Anti-TIGIT | YIDPYNFGAKYNEKFG |
| | antibody, VH- | |
| | CDR2 variant | |
| 63 | Anti-TIGIT | YIDPYNSGAKYNEKFG |
| | antibody, VH- | |
| | CDR2 variant | |
| 64 | Anti-TIGIT | YIDPYNYGAKYNEKFG |
| | antibody, VH- | |
| | CDR2 variant | |
| 65 | Anti-TIGIT | YIDPYNVGAKYNEKFG |
| | antibody, VH- | |
| | CDR2 variant | |
| 66 | Anti-TIGIT | YIDPYNDRAKYNEKFKG |
| | antibody, VH- | |
| | CDR2 variant | |
| 67 | Anti-TIGIT | YIDPYNDNAKYNEKFKG |
| | antibody, VH- | |
| | CDR2 variant | |
| 68 | Anti-TIGIT | YIDPYNDQAKYNEKFKG |
| | antibody, VH- | |
| | CDR2 variant | |
| 69 | Anti-TIGIT | YIDPYNDEAKYNEKFKG |
| | antibody, VH- | |
| | CDR2 variant | |
| 70 | Anti-TIGIT | YIDPYNDLAKYNEKFKG |
| | antibody, VH- | |
| | CDR2 variant | |
| 71 | Anti-TIGIT | YIDPYNDKAKYNEKFKG |
| | antibody, VH- | |
| | CDR2 variant | |
| 72 | Anti-TIGIT | YIDPYNDSAKYNEKFKG |
| | antibody, VH- | |
| | CDR2 variant | |
| 73 | Anti-TIGIT | YIDPYNDYAKYNEKFKG |
| | antibody, VH- | |
| | CDR2 variant | |
| 74 | Anti-TIGIT | YIDPYNDVAKYNEKFKG |
| | antibody, VH- | |
| | CDR2 variant | |
| 75 | Anti-TIGIT | YIDPYNDGAKYSQKFQG |
| | antibody, VH- | |
| | CDR2 variant | |
| 76 | Anti-TIGIT | YIDPYNX₇X₈AKYX₁₂X₁₃KFX₁₆G |
| | antibody, VH- | X₇ = D, R, L, K, F, S, Y or V |
| | CDR2 | X₈ = G, R, N, Q, E, L K, S, Y or V |
| | consensus | X₁₂ = N, A or S |
| | | X₁₃ = E or Q |
| | | X₁₆ = K or Q |
| 77 | Anti-TIGIT | GGPYGAYFDV |
| | antibody, VH- | |
| | CDR3 variant | |
| 78 | Anti-TIGIT | GGPYGDYFDV |
| | antibody, VH- | |
| | CDR3 variant | |
| 79 | Anti-TIGIT | GGPYGEYFDV |
| | antibody, VH- | |
| | CDR3 variant | |
| 80 | Anti-TIGIT | GGPYGFYFDV |
| | antibody, VH- | |
| | CDR3 variant | |
| 81 | Anti-TIGIT | GGPYGGYFDV |
| | antibody, VH- | |
| | CDR3 variant | |
| 82 | Anti-TIGIT | GGPYGIYFDV |
| | antibody, VH- | |
| | CDR3 variant | |
| 83 | Anti-TIGIT | GGPYGKYFDV |
| | antibody, VH- | |
| | CDR3 variant | |
| 84 | Anti-TIGIT | GGPYGNYFDV |
| | antibody, VH- | |
| | CDR3 variant | |
| 85 | Anti-TIGIT | GGPYGQYFDV |
| | antibody, VH- | |
| | CDR3 variant | |
| 86 | Anti-TIGIT | GGPYGRYFDV |
| | antibody, VH- | |
| | CDR3 variant | |
| 87 | Anti-TIGIT | GGPYGSYFDV |
| | antibody, VH- | |
| | CDR3 variant | |
| 88 | Anti-TIGIT | GGPYGTYFDV |
| | antibody, VH- | |
| | CDR3 variant | |
| 89 | Anti-TIGIT | GGPYGVYFDV |
| | antibody, VH- | |
| | CDR3 variant | |
| 90 | Anti-TIGIT | GGPYGYYFDV |
| | antibody, VH- | |
| | CDR3 variant | |
| 91 | Anti-TIGIT | GGPYGX₆YFDV |
| | antibody, VH- | X₆ = W, A, D, E, F, G, I, K, N, Q, R, S, T, V or Y |
| | CDR3 | |
| | consensus | |
| 92 | Anti-TIGIT | AAKTLAE |
| | antibody, VL- | |
| | CDR2 variant | |
| 93 | Anti-TIGIT | YAKTLAE |
| | antibody, VL- | |
| | CDR2 variant | |
| 94 | Anti-TIGIT | WAKTLAE |
| | antibody, VL- | |
| | CDR2 variant | |
| 95 | Anti-TIGIT | SAKTLAE |
| | antibody, VL- | |
| | CDR2 variant | |
| 96 | Anti-TIGIT | TAKTLAE |
| | antibody, VL- | |
| | CDR2 variant | |
| 97 | Anti-TIGIT | IAKTLAE |
| | antibody, VL- | |
| | CDR2 variant | |
| 98 | Anti-TIGIT | VAKTLAE |
| | antibody, VL- | |
| | CDR2 variant | |
| 99 | Anti-TIGIT | NNKTLAE |
| | antibody, VL- | |
| | CDR2 variant | |
| 100 | Anti-TIGIT | NIKTLAE |
| | antibody, VL- | |
| | CDR2 variant | |
| 101 | Anti-TIGIT | NLLTLAE |
| | antibody, VL- | |
| | CDR2 variant | |
| 102 | Anti-TIGIT | NTKTLAE |
| | antibody, VL- | |
| | CDR2 variant | |
| 103 | Anti-TIGIT | NVKTLAE |
| | antibody, VL- | |
| | CDR2 variant | |
| 104 | Anti-TIGIT | X₁X₂KTLAE |
| | antibody, VL- | X₁ = N, A, V, W, S, T, R, H G, I or V |
| | CDR2 | X₂ = A, N, I, L, T or V |
| | consensus | |

1. A method of treating cancer, comprising administering to a human patient in need thereof:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof,
wherein optionally the anti-human PD-1 monoclonal antibody is a human antibody or a humanized antibody;
wherein optionally the anti-human LAG3 monoclonal antibody is a human antibody or a humanized antibody; and
wherein optionally the anti-human TIGIT monoclonal antibody is a human antibody or a humanized antibody.

2. The method of claim 1, wherein the cancer is selected from the group consisting of osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer, non-small cell lung cancer, gastric cancer, colorectal cancer, cervical cancer, synovial sarcoma, head and neck cancer, squamous cell carcinoma, lymphoma, diffuse large B-cell lymphoma, non-Hodgkin lymphoma, multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, and carcinoid cancer.

3. A method of enhancing T cell activity, comprising contacting the T cells with:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof,
wherein optionally the anti-human PD-1 monoclonal antibody is a human antibody or a humanized antibody;
wherein optionally the anti-human LAG3 monoclonal antibody is a human antibody or a humanized antibody; and
wherein optionally the anti-human TIGIT monoclonal antibody is a human antibody or a humanized antibody.

4-6. (canceled)

7. A pharmaceutical composition, or a kit, comprising:
(a) an anti-human PD-1 monoclonal antibody or antigen binding fragment thereof;
(b) an anti-human LAG3 monoclonal antibody or antigen binding fragment thereof; and
(c) an anti-human TIGIT monoclonal antibody or antigen binding fragment thereof,
wherein optionally the anti-human PD-1 monoclonal antibody is a human antibody or a humanized antibody;
wherein optionally the anti-human LAG3 monoclonal antibody is a human antibody or a humanized antibody; and
wherein optionally the anti-human TIGIT monoclonal antibody is a human antibody or a humanized antibody.

8-18. (canceled)

19. The method of claim 1, wherein the anti-human PD-1 monoclonal antibody or antigen binding fragment thereof
(i) comprises a light chain variable region (V_{L}) complementarity determining region 1 (CDR1), a V_{L} CDR2, and a V_{L} CDR3 comprising amino acid sequences as set forth in SEQ ID NOS:1, 2, and 3, respectively, and a heavy chain variable region (V_{H}) CDR1, a V_{H} CDR2, and a V_{H} CDR3 comprising amino acid sequences as set forth in SEQ ID NOS:6, 7, and 8, respectively;
(ii) comprises a V_{L} region comprising an amino acid sequence as set forth in SEQ ID NO:4, and a V_{H} region comprising an amino acid sequence as set forth in SEQ ID NO:9; (iii) a light chain comprising or consisting of an amino acid sequence as set forth in SEQ ID NO:5 and a heavy chain comprising or consisting of an amino acid sequence as set forth in SEQ ID NO:10;
(iv) comprises a light chain variable region (V_{L}) complementarity determining region 1 (CDR1), a V_{L} CDR2, and a V_{L} CDR3 comprising amino acid sequences as set forth in SEQ ID NOS:31, 32, and 33, respectively, and a heavy chain variable region (V_{H}) CDR1, a V_{H} CDR2, and a V_{H} CDR3 comprising amino acid sequences as set forth in SEQ ID NOS:36, 37, and 38, respectively;
(v) comprises a V_{L} region comprising an amino acid sequence as set forth in SEQ ID NO:34, and a V_{H} region comprising an amino acid sequence as set forth in SEQ ID NO:39;
(vi) comprises a light chain comprising or consisting of an amino acid sequence as set forth in SEQ ID NO:35 and a heavy chain comprising or consisting of an amino acid sequence as set forth in SEQ ID NO:40;
(vii) is pembrolizumab;
(viii) is nivolumab; or
(ix) is cemiplimab.

20-27. (canceled)

28. The method of claim 1, wherein the anti-human LAG3 monoclonal antibody or antigen binding fragment thereof comprises:
(i) a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively;
(ii) a V_{L} region comprising an amino acid sequence as set forth in SEQ ID NO:14, and a V_{H} region comprising an amino acid sequence as set forth in SEQ ID NO:19; or
(iii) a light chain comprising or consisting of an amino acid sequence as set forth in SEQ ID NO:15 and a heavy chain comprising or consisting of an amino acid sequence as set forth in SEQ ID NO:20.

29-30. (canceled)

31. The method of claim 1, wherein the anti-human TIGIT monoclonal antibody or antigen binding fragment thereof comprises:
(i) a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively;
(ii) a V_{L} region comprising an amino acid sequence as set forth in SEQ ID NO:24, and a V_{H} region comprising an amino acid sequence as set forth in SEQ ID NO:29; or
(iii) a light chain comprising or consisting of an amino acid sequence as set forth in SEQ ID NO:25 and a heavy chain comprising or consisting of an amino acid sequence as set forth in SEQ ID NO:30.

32-33. (canceled)

34. The method of claim 1, wherein:
(i) (a) the anti-human PD-1 monoclonal antibody or antigen binding fragment thereof comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively;
(b) the anti-human LAG3 monoclonal antibody or antigen binding fragment thereof comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) the anti-human TIGIT monoclonal antibody or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively;
(ii) (a) the anti-human PD-1 monoclonal antibody or antigen binding fragment thereof comprises a V_{L} region comprising an amino acid sequence as set forth in SEQ ID NO:4, and a V_{H} region comprising an amino acid sequence as set forth in SEQ ID NO:9;
(b) the anti-human LAG3 monoclonal antibody or antigen binding fragment thereof comprises a V_{L} region comprising an amino acid sequence as set forth in SEQ ID NO:14, and a V_{H} region comprising an amino acid sequence as set forth in SEQ ID NO:19; and
(c) the anti-human TIGIT monoclonal antibody or antigen binding fragment thereof comprises a V_{L} region comprising an amino acid sequence as set forth in SEQ ID NO:24, and a V_{H} region comprising an amino acid sequence as set forth in SEQ ID NO:29;
(iii) (a) the anti-human PD-1 monoclonal antibody or antigen binding fragment thereof comprises a light chain comprising or consisting of an amino acid sequence as set forth in SEQ ID NO:5 and a heavy chain comprising or consisting of an amino acid sequence as set forth in SEQ ID NO:10;
(b) the anti-human LAG3 monoclonal antibody or antigen binding fragment thereof comprises a light chain comprising or consisting of an amino acid sequence as set forth in SEQ ID NO:15 and a heavy chain comprising or consisting of an amino acid sequence as set forth in SEQ ID NO:20; and
(c) the anti-human TIGIT monoclonal antibody or antigen binding fragment thereof comprises a light chain comprising or consisting of an amino acid sequence as set forth in SEQ ID NO:25 and a heavy chain comprising or consisting of an amino acid sequence as set forth in SEQ ID NO:30;
(iv) (a) the anti-human PD-1 monoclonal antibody or antigen binding fragment thereof comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively;
(b) the anti-human LAG3 monoclonal antibody or antigen binding fragment thereof comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) the anti-human TIGIT monoclonal antibody or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively;
(v) (a) the anti-human PD-1 monoclonal antibody or antigen binding fragment thereof comprises a V_{L} region comprising an amino acid sequence as set forth in SEQ ID NO:34, and a V_{H} region comprising an amino acid sequence as set forth in SEQ ID NO:39;
(b) the anti-human LAG3 monoclonal antibody or antigen binding fragment thereof comprises a V_{L} region comprising an amino acid sequence as set forth in SEQ ID NO:14, and a V_{H} region comprising an amino acid sequence as set forth in SEQ ID NO:19; and
(c) the anti-human TIGIT monoclonal antibody or antigen binding fragment thereof comprises a V_{L} region comprising an amino acid sequence as set forth in SEQ ID NO:24, and a V_{H} region comprising an amino acid sequence as set forth in SEQ ID NO:29; or
(vi) (a) the anti-human PD-1 monoclonal antibody or antigen binding fragment thereof comprises a light chain comprising or consisting of an amino acid sequence as set forth in SEQ ID NO:35 and a heavy chain comprising or consisting of an amino acid sequence as set forth in SEQ ID NO:40;
(b) the anti-human LAG3 monoclonal antibody or antigen binding fragment thereof comprises a light chain comprising or consisting of an amino acid sequence as set forth in SEQ ID NO:15 and a heavy chain comprising or consisting of an amino acid sequence as set forth in SEQ ID NO:20; and
(c) the anti-human TIGIT monoclonal antibody or antigen binding fragment thereof comprises a light chain comprising or consisting of an amino acid sequence as set forth in SEQ ID NO:25 and a heavy chain comprising or consisting of an amino acid sequence as set forth in SEQ ID NO:30.

35-39. (canceled)

40. The method of claim 19(vii), wherein the human patient is administered 200 mg, 240 mg, or 2 mg/kg pembrolizumab, and wherein pembrolizumab is administered once every three weeks.

41. The method of claim 19(vii), wherein the human patient is administered 400 mg pembrolizumab, and wherein pembrolizumab is administered once every six weeks.

42. The method of claim 19(viii) wherein the human patient is administered 240 mg or 3 mg/kg nivolumab, and wherein nivolumab is administered once every two weeks.

43. The method of claim 19(ix) wherein the human patient is administered 350 mg cemiplimab, and wherein cemiplimab is administered once every three weeks.


SEQUENCE LISTING (104 sequences)

SEQ ID NO: 1 - protein, 15 aa, synthetic construct
  REGION 1..15: note = Pembrolizumab, VL-CDR2
RASKGVSTSGYSYLH

SEQ ID NO: 2 - protein, 7 aa, synthetic construct
  REGION 1..7: note = Pembrolizumab, VL-CDR2
LASYLES

SEQ ID NO: 3 - protein, 9 aa, synthetic construct
  REGION 1..9: note = Pembrolizumab, VL-CDR3
QHSRDLPLT

SEQ ID NO: 4 - protein, 111 aa, synthetic construct [sequence omitted]

SEQ ID NO: 5 - protein, 218 aa, synthetic construct
  REGION 1..218: note = Pembrolizumab, light chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 5 aa, synthetic construct
  REGION 1..5: note = Pembrolizumab, VH-CDR1
NYYMY

SEQ ID NO: 7 - protein, 17 aa, synthetic construct
  REGION 1..17: note = Pembrolizumab, VH-CDR2
GINPSNGGTNFNEKFKN

SEQ ID NO: 8 - protein, 11 aa, synthetic construct
  REGION 1..11: note = Pembrolizumab, VH-CDR3
RDYRFDMGFDY

SEQ ID NO: 9 - protein, 120 aa, synthetic construct [sequence omitted]

SEQ ID NO: 10 - protein, 447 aa, synthetic construct
  REGION 1..447: note = Pembrolizumab, heavy chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 15 aa, synthetic construct
  REGION 1..15: note = Anti-LAG3 Ab6, VL-CDR1
KASQSLDYEGDSDMN

SEQ ID NO: 12 - protein, 7 aa, synthetic construct
  REGION 1..7: note = Anti-LAG3 Ab6, VL-CDR2
GASNLES

SEQ ID NO: 13 - protein, 9 aa, synthetic construct
  REGION 1..9: note = Anti-LAG3 Ab6, VL-CDR3
QQSTEDPRT

SEQ ID NO: 14 - protein, 111 aa, synthetic construct
  REGION 1..111: note = Anti-LAG3 Ab6, VL
DIVMTQTPLSLSVTPGQPASISCKASQSLDYEGDSDMNWYLQKPGQPPQLLIYGASNLESGVPDRFSGSGSGTDFTLKISRVEAEDVGVYYCQQSTEDPRTFGGGTKVEIK

SEQ ID NO: 15 - protein, 218 aa, synthetic construct
  REGION 1..218: note = Anti-LAG3 Ab6, light chain
DIVMTQTPLSLSVTPGQPASISCKASQSLDYEGDSDMNWYLQKPGQPPQLLIYGASNLESGVPDRFSGSGSGTDFTLKISRVEAEDVGVYYCQQSTEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 5 aa, synthetic construct
  REGION 1..5: note = Anti-LAG3 Ab6, VH-CDR1
DYNVD

SEQ ID NO: 17 - protein, 17 aa, synthetic construct
  REGION 1..17: note = Anti-LAG3 Ab6, VH-CDR2
DINPNDGGTIYAQKFQE

SEQ ID NO: 18 - protein, 10 aa, synthetic construct
  REGION 1..10: note = Anti-LAG3 Ab6, VH-CDR3
NYRWFGAMDH

SEQ ID NO: 19 - protein, 119 aa, synthetic construct
  REGION 1..119: note = Anti-LAG3 Ab6, VH
QMQLVQSGPEVKKPGTSVKVSCKASGYTFTDYNVDWVRQARGQRLEWIGDINPNDGGTIYAQKFQERVTITVDKSTSTAYMELSSLRSEDTAVYYCARNYRWFGAMDHWGQGTTVTVSS

SEQ ID NO: 20 - protein, 446 aa, synthetic construct
  REGION 1..446: note = Anti-LAG3 Ab6, heavy chain
QMQLVQSGPEVKKPGTSVKVSCKASGYTFTDYNVDWVRQARGQRLEWIGDINPNDGGTIYAQKFQERVTITVDKSTSTAYMELSSLRSEDTAVYYCARNYRWFGAMDHWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 11 aa, synthetic construct
  REGION 1..11: note = Anti-TIGIT antibody, VL-CDR1
RASEHIYSYLS

SEQ ID NO: 22 - protein, 7 aa, synthetic construct
  REGION 1..7: note = Anti-TIGIT antibody, VL-CDR2
NAKTLAE

SEQ ID NO: 23 - protein, 9 aa, synthetic construct
  REGION 1..9: note = Anti-TIGIT antibody, VL-CDR3
QHHFGSPLT

SEQ ID NO: 24 - protein, 107 aa, synthetic construct
  REGION 1..107: note = Anti-TIGIT antibody, VL
DIQMTQSPSSLSASVGDRVTITCRASEHIYSYLSWYQQKPGKVPKLLIYNAKTLAEGVPSRFSGSGSGTDFTLTISSLQPEDVATYYCQHHFGSPLTFGQGTRLEIK

SEQ ID NO: 25 - protein, 214 aa, synthetic construct
  REGION 1..214: note = Anti-TIGIT antibody, light chain
DIQMTQSPSSLSASVGDRVTITCRASEHIYSYLSWYQQKPGKVPKLLIYNAKTLAEGVPSRFSGSGSGTDFTLTISSLQPEDVATYYCQHHFGSPLTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 5 aa, synthetic construct
  REGION 1..5: note = Anti-TIGIT antibody, VH-CDR1
SYVMH

SEQ ID NO: 27 - protein, 17 aa, synthetic construct
  REGION 1..17: note = Anti-TIGIT antibody, VH-CDR2
YIDPYNDGAKYAQKFQG

SEQ ID NO: 28 - protein, 10 aa, synthetic construct
  REGION 1..10: note = Anti-TIGIT antibody, VH-CDR3
GGPYGWYFDV

SEQ ID NO: 29 - protein, 119 aa, synthetic construct
  REGION 1..119: note = Anti-TIGIT antibody, VH
EVQLVQSGAEVKKPGSSVKVSCKASGYTFSSYVMHWVRQAPGQGLEWIGYIDPYNDGAKYAQKFQGRVTLTSDKSTSTAYMELSSLRSEDTAVYYCARGGPYGWYFDVWGQGTTVTVSS

SEQ ID NO: 30 - protein, 449 aa, synthetic construct
  REGION 1..449: note = Anti-TIGIT antibody, heavy chain
EVQLVQSGAEVKKPGSSVKVSCKASGYTFSSYVMHWVRQAPGQGLEWIGYIDPYNDGAKYAQKFQGRVTLTSDKSTSTAYMELSSLRSEDTAVYYCARGGPYGWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 11 aa, synthetic construct
  REGION 1..11: note = Nivolumab, VL-CDR1
RASQSVSSYLA

SEQ ID NO: 32 - protein, 7 aa, synthetic construct
  REGION 1..7: note = Nivolumab, VL-CDR2
DASNRAT

SEQ ID NO: 33 - protein, 9 aa, synthetic construct
  REGION 1..9: note = Nivolumab, VL-CDR3
QQSSNWPRT

SEQ ID NO: 34 - protein, 107 aa, synthetic construct [sequence omitted]

SEQ ID NO: 35 - protein, 214 aa, synthetic construct
  REGION 1..214: note = Nivolumab, light chain
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 5 aa, synthetic construct
  REGION 1..5: note = Nivolumab, VH-CDR1
NSGMH

SEQ ID NO: 37 - protein, 17 aa, synthetic construct
  REGION 1..17: note = Nivolumab, VH-CDR2
VIWYDGSKRYYADSVKG

SEQ ID NO: 38 - protein, 4 aa, synthetic construct
  REGION 1..4: note = Nivolumab, VH-CDR3
NDDY

SEQ ID NO: 39 - protein, 113 aa, synthetic construct [sequence omitted]

SEQ ID NO: 40 - protein, 440 aa, synthetic construct
  REGION 1..440: note = Nivolumab, heavy chain
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 17 aa, synthetic construct
  REGION 1..17: note = Anti-LAG3 Ab9, VH-CDR2
DINPNGGGTIYAQKFQE

SEQ ID NO: 42 - protein, 17 aa, synthetic construct
  REGION 1..17: note = Anti-LAG3 Ab8, VH-CDR2
DINPNQGGTIYAQKFQE

SEQ ID NO: 43 - protein, 17 aa, synthetic construct
  REGION 1..17: note = Anti-LAG3 Ab7, VH-CDR2
DINPNSGGTIYAQKFQE

SEQ ID NO: 44 - protein, 17 aa, synthetic construct
  REGION 1..17: note = Anti-LAG3 Ab5, VH-CDR2
DINPNNGGTIYAQKFQE

SEQ ID NO: 45 - protein, 119 aa, synthetic construct
  REGION 1..119: note = Anti-LAG3 Ab9, VH
QMQLVQSGPEVKKPGTSVKVSCKASGYTFTDYNVDWVRQARGQRLEWIGDINPNGGGTIYAQKFQERVTITVDKSTSTAYMELSSLRSEDTAVYYCARNYRWFGAMDHWGQGTTVTVSS

SEQ ID NO: 46 - protein, 119 aa, synthetic construct
  REGION 1..119: note = Anti-LAG3 Ab8, VH
QMQLVQSGPEVKKPGTSVKVSCKASGYTFTDYNVDWVRQARGQRLEWIGDINPNQGGTIYAQKFQERVTITVDKSTSTAYMELSSLRSEDTAVYYCARNYRWFGAMDHWGQGTTVTVSS

SEQ ID NO: 47 - protein, 119 aa, synthetic construct
  REGION 1..119: note = Anti-LAG3 Ab7, VH
QMQLVQSGPEVKKPGTSVKVSCKASGYTFTDYNVDWVRQARGQRLEWIGDINPNSGGTIYAQKFQERVTITVDKSTSTAYMELSSLRSEDTAVYYCARNYRWFGAMDHWGQGTTVTVSS

SEQ ID NO: 48 - protein, 119 aa, synthetic construct
  REGION 1..119: note = Anti-LAG3 Ab5, VH
QMQLVQSGPEVKKPGTSVKVSCKASGYTFTDYNVDWVRQARGQRLEWIGDINPNNGGTIYAQKFQERVTITVDKSTSTAYMELSSLRSEDTAVYYCARNYRWFGAMDHWGQGTTVTVSS

SEQ ID NO: 49 - protein, 449 aa, synthetic construct
  REGION 1..449: note = Anti-LAG3 variant, IgG1 HC with VH-CDR2 of Ab9
QMQLVQSGPEVKKPGTSVKVSCKASGYTFTDYNVDWVRQARGQRLEWIGDINPNGGGTIYAQKFQERVTITVDKSTSTAYMELSSLRSEDTAVYYCARNYRWFGAMDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 449 aa, synthetic construct
  REGION 1..449: note = Anti-LAG3 Ab4, IgG1 HC
QMQLVQSGPEVKKPGTSVKVSCKASGYTFTDYNVDWVRQARGQRLEWIGDINPNQGGTIYAQKFQERVTITVDKSTSTAYMELSSLRSEDTAVYYCARNYRWFGAMDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 449 aa, synthetic construct
  REGION 1..449: note = Anti-LAG3 Ab2, IgG1 HC
QMQLVQSGPEVKKPGTSVKVSCKASGYTFTDYNVDWVRQARGQRLEWIGDINPNSGGTIYAQKFQERVTITVDKSTSTAYMELSSLRSEDTAVYYCARNYRWFGAMDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 449 aa, synthetic construct
  REGION 1..449: note = Anti-LAG3 Ab3, IgG1 HC
QMQLVQSGPEVKKPGTSVKVSCKASGYTFTDYNVDWVRQARGQRLEWIGDINPNDGGTIYAQKFQERVTITVDKSTSTAYMELSSLRSEDTAVYYCARNYRWFGAMDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 449 aa, synthetic construct
  REGION 1..449: note = Anti-LAG3 Ab1, IgG1 HC
QMQLVQSGPEVKKPGTSVKVSCKASGYTFTDYNVDWVRQARGQRLEWIGDINPNNGGTIYAQKFQERVTITVDKSTSTAYMELSSLRSEDTAVYYCARNYRWFGAMDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 446 aa, synthetic construct
  REGION 1..446: note = Anti-LAG3 Ab9, IgG4-S228P HC
QMQLVQSGPEVKKPGTSVKVSCKASGYTFTDYNVDWVRQARGQRLEWIGDINPNGGGTIYAQKFQERVTITVDKSTSTAYMELSSLRSEDTAVYYCARNYRWFGAMDHWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 55 - protein, 446 aa, synthetic construct
  REGION 1..446: note = Anti-LAG3 Ab8, IgG4-S228P HC
QMQLVQSGPEVKKPGTSVKVSCKASGYTFTDYNVDWVRQARGQRLEWIGDINPNQGGTIYAQKFQERVTITVDKSTSTAYMELSSLRSEDTAVYYCARNYRWFGAMDHWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 56 - protein, 446 aa, synthetic construct
  REGION 1..446: note = Anti-LAG3 Ab7, IgG4-S228P HC
QMQLVQSGPEVKKPGTSVKVSCKASGYTFTDYNVDWVRQARGQRLEWIGDINPNSGGTIYAQKFQERVTITVDKSTSTAYMELSSLRSEDTAVYYCARNYRWFGAMDHWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 446 aa, synthetic construct
  REGION 1..446: note = Anti-LAG3 Ab5, IgG4-S228P HC
QMQLVQSGPEVKKPGTSVKVSCKASGYTFTDYNVDWVRQARGQRLEWIGDINPNNGGTIYAQKFQERVTITVDKSTSTAYMELSSLRSEDTAVYYCARNYRWFGAMDHWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 58 - protein, 17 aa, synthetic construct
  REGION 1..17: note = Anti-TIGIT antibody, VH-CDR2 variant
YIDPYNDGAKYNEKFKG

SEQ ID NO: 59 - protein, 16 aa, synthetic construct
  REGION 1..16: note = Anti-TIGIT antibody, VH-CDR2 variant
YIDPYNRGAKYNEKFG

SEQ ID NO: 60 - protein, 16 aa, synthetic construct
  REGION 1..16: note = Anti-TIGIT antibody, VH-CDR2 variant
YIDPYNLGAKYNEKFG

SEQ ID NO: 61 - protein, 16 aa, synthetic construct
  REGION 1..16: note = Anti-TIGIT antibody, VH-CDR2 variant
YIDPYNKGAKYNEKFG

SEQ ID NO: 62 - protein, 16 aa, synthetic construct
  REGION 1..16: note = Anti-TIGIT antibody, VH-CDR2 variant
YIDPYNFGAKYNEKFG

SEQ ID NO: 63 - protein, 16 aa, synthetic construct
  REGION 1..16: note = Anti-TIGIT antibody, VH-CDR2 variant
YIDPYNSGAKYNEKFG

SEQ ID NO: 64 - protein, 16 aa, synthetic construct
  REGION 1..16: note = Anti-TIGIT antibody, VH-CDR2 variant
YIDPYNYGAKYNEKFG

SEQ ID NO: 65 - protein, 16 aa, synthetic construct
  REGION 1..16: note = Anti-TIGIT antibody, VH-CDR2 variant
YIDPYNVGAKYNEKFG

SEQ ID NO: 66 - protein, 17 aa, synthetic construct
  REGION 1..17: note = Anti-TIGIT antibody, VH-CDR2 variant
YIDPYNDRAKYNEKFKG

SEQ ID NO: 67 - protein, 17 aa, synthetic construct
  REGION 1..17: note = Anti-TIGIT antibody, VH-CDR2 variant
YIDPYNDNAKYNEKFKG

SEQ ID NO: 68 - protein, 17 aa, synthetic construct
  REGION 1..17: note = Anti-TIGIT antibody, VH-CDR2 variant
YIDPYNDQAKYNEKFKG

SEQ ID NO: 69 - protein, 17 aa, synthetic construct
  REGION 1..17: note = Anti-TIGIT antibody, VH-CDR2 variant
YIDPYNDEAKYNEKFKG

SEQ ID NO: 70 - protein, 17 aa, synthetic construct
  REGION 1..17: note = Anti-TIGIT antibody, VH-CDR2 variant
YIDPYNDLAKYNEKFKG

SEQ ID NO: 71 - protein, 17 aa, synthetic construct
  REGION 1..17: note = Anti-TIGIT antibody, VH-CDR2 variant
YIDPYNDKAKYNEKFKG

SEQ ID NO: 72 - protein, 17 aa, synthetic construct
  REGION 1..17: note = Anti-TIGIT antibody, VH-CDR2 variant
YIDPYNDSAKYNEKFKG

SEQ ID NO: 73 - protein, 17 aa, synthetic construct
  REGION 1..17: note = Anti-TIGIT antibody, VH-CDR2 variant
YIDPYNDYAKYNEKFKG

SEQ ID NO: 74 - protein, 17 aa, synthetic construct
  REGION 1..17: note = Anti-TIGIT antibody, VH-CDR2 variant
YIDPYNDVAKYNEKFKG

SEQ ID NO: 75 - protein, 17 aa, synthetic construct
  REGION 1..17: note = Anti-TIGIT antibody, VH-CDR2 variant
YIDPYNDGAKYSQKFQG

SEQ ID NO: 76 - protein, 17 aa, synthetic construct
  REGION 1..17: note = Anti-TIGIT antibody, VH-CDR2 consensus
  VARIANT 7: note = Asp, Arg, Leu, Lys, Phe, Ser, Tyr, or Val
  VARIANT 8: note = Gly, Arg, Asn, Gln, Glu, Leu, Lys, Ser, Tyr, or Val
  VARIANT 12: note = Asn, Ala, or Ser
  VARIANT 13: note = Glu or Gln
  VARIANT 16: note = Lys or Gln
YIDPYNXXAKYXXKFXG

SEQ ID NO: 77 - protein, 10 aa, synthetic construct
  REGION 1..10: note = Anti-TIGIT antibody, VH-CDR3 variant
GGPYGAYFDV

SEQ ID NO: 78 - protein, 10 aa, synthetic construct
  REGION 1..10: note = Anti-TIGIT antibody, VH-CDR3 variant
GGPYGDYFDV

SEQ ID NO: 79 - protein, 10 aa, synthetic construct
  REGION 1..10: note = Anti-TIGIT antibody, VH-CDR3 variant
GGPYGEYFDV

SEQ ID NO: 80 - protein, 10 aa, synthetic construct
  REGION 1..10: note = Anti-TIGIT antibody, VH-CDR3 variant
GGPYGFYFDV

SEQ ID NO: 81 - protein, 10 aa, synthetic construct
  REGION 1..10: note = Anti-TIGIT antibody, VH-CDR3 variant
GGPYGGYFDV

SEQ ID NO: 82 - protein, 10 aa, synthetic construct
  REGION 1..10: note = Anti-TIGIT antibody, VH-CDR3 variant
GGPYGIYFDV

SEQ ID NO: 83 - protein, 10 aa, synthetic construct
  REGION 1..10: note = Anti-TIGIT antibody, VH-CDR3 variant
GGPYGKYFDV

SEQ ID NO: 84 - protein, 10 aa, synthetic construct
  REGION 1..10: note = Anti-TIGIT antibody, VH-CDR3 variant
GGPYGNYFDV

SEQ ID NO: 85 - protein, 10 aa, synthetic construct
  REGION 1..10: note = Anti-TIGIT antibody, VH-CDR3 variant
GGPYGQYFDV

SEQ ID NO: 86 - protein, 10 aa, synthetic construct
  REGION 1..10: note = Anti-TIGIT antibody, VH-CDR3 variant
GGPYGRYFDV

SEQ ID NO: 87 - protein, 10 aa, synthetic construct
  REGION 1..10: note = Anti-TIGIT antibody, VH-CDR3 variant
GGPYGSYFDV

SEQ ID NO: 88 - protein, 10 aa, synthetic construct
  REGION 1..10: note = Anti-TIGIT antibody, VH-CDR3 variant
GGPYGTYFDV

SEQ ID NO: 89 - protein, 10 aa, synthetic construct
  REGION 1..10: note = Anti-TIGIT antibody, VH-CDR3 variant
GGPYGVYFDV

SEQ ID NO: 90 - protein, 10 aa, synthetic construct
  REGION 1..10: note = Anti-TIGIT antibody, VH-CDR3 variant
GGPYGYYFDV

SEQ ID NO: 91 - protein, 10 aa, synthetic construct
  REGION 1..10: note = Anti-TIGIT antibody, VH-CDR3 consensus
  VARIANT 6: note = Trp, Ala, Asp, Glu, Phe, Gly, Ile, Lys, Asn, Gln, Arg, Ser, Thr, Val, or Tyr
GGPYGXYFDV

SEQ ID NO: 92 - protein, 7 aa, synthetic construct
  REGION 1..7: note = Anti-TIGIT antibody, VL-CDR2 variant
AAKTLAE

SEQ ID NO: 93 - protein, 7 aa, synthetic construct
  REGION 1..7: note = Anti-TIGIT antibody, VL-CDR2 variant
YAKTLAE

SEQ ID NO: 94 - protein, 7 aa, synthetic construct
  REGION 1..7: note = Anti-TIGIT antibody, VL-CDR2 variant
WAKTLAE

SEQ ID NO: 95 - protein, 7 aa, synthetic construct
  REGION 1..7: note = Anti-TIGIT antibody, VL-CDR2 variant
SAKTLAE

SEQ ID NO: 96 - protein, 7 aa, synthetic construct
  REGION 1..7: note = Anti-TIGIT antibody, VL-CDR2 variant
TAKTLAE

SEQ ID NO: 97 - protein, 7 aa, synthetic construct
  REGION 1..7: note = Anti-TIGIT antibody, VL-CDR2 variant
IAKTLAE

SEQ ID NO: 98 - protein, 7 aa, synthetic construct
  REGION 1..7: note = Anti-TIGIT antibody, VL-CDR2 variant
VAKTLAE

SEQ ID NO: 99 - protein, 7 aa, synthetic construct
  REGION 1..7: note = Anti-TIGIT antibody, VL-CDR2 variant
NNKTLAE

SEQ ID NO: 100 - protein, 7 aa, synthetic construct
  REGION 1..7: note = Anti-TIGIT antibody, VL-CDR2 variant
NIKTLAE

SEQ ID NO: 101 - protein, 7 aa, synthetic construct
  REGION 1..7: note = Anti-TIGIT antibody, VL-CDR2 variant
NLLTLAE

SEQ ID NO: 102 - protein, 7 aa, synthetic construct
  REGION 1..7: note = Anti-TIGIT antibody, VL-CDR2 variant
NTKTLAE

SEQ ID NO: 103 - protein, 7 aa, synthetic construct
  REGION 1..7: note = Anti-TIGIT antibody, VL-CDR2 variant
NVKTLAE

SEQ ID NO: 104 - protein, 7 aa, synthetic construct
  REGION 1..7: note = Anti-TIGIT antibody, VL-CDR2 consensus
  VARIANT 1: note = Asn, Ala, Val, Trp, Ser, Thr, Arg, His, Gly, Ile, or Val
  VARIANT 2: note = Ala, Asn, Ile, Leu, Thr, or Val
XXKTLAE